(19) United States
(12) Patent Application Publication
(10) Pub. No.: US 2022/0177418 A1
(43) Pub. Date: Jun. 9, 2022

(54) INHIBITORS OF THE N-TERMINAL DOMAIN OF THE ANDROGEN RECEPTOR

(71) Applicants: The Regents of the University of California, Oakland, CA (US); The United States Government represented by the Department of Veterans Affairs, Washington, DC (US)

(72) Inventors: Matthew Rettig, Los Angeles, CA (US); Michael E. Jung, Los Angeles, CA (US); D. Elshan Nakath G. Ralalage, Los Angeles, CA (US); Jiabin An, Los Angeles, CA (US)

(21) Appl. No.: 17/599,083

(22) PCT Filed: Mar. 27, 2020
(86) PCT No.: PCT/US20/25120

§ 371 Date: Sep. 28, 2021

Related U.S. Application Data
(60) Provisional application No. 62/826,636, filed on Mar. 29, 2019.

(51) Int. Cl. C07C 233/13; C07C 233/40; C07C 233/91; C07C 235/76; C07C 255/46; C07C 307/04; C07C 49/537; C07C 49/697; C07C 69/618; C07D 207/273; C07D 207/38; C07D 211/70; C07D 213/46; C07D 307/46; C07D 303/32; C07D 263/32

(52) U.S. Cl. CPC C07C 233/13; C07C 2601/02; C07C 233/91; C07C 235/76; C07C 255/46; C07C 307/04; C07C 49/537; C07C 49/697; C07C 69/618; C07D 207/273; C07D 207/38; C07D 211/70; C07D 213/46; C07D 307/46; C07D 303/32; C07D 263/32; C07C 233/40

(57) ABSTRACT

The present disclosure provides compounds and methods for inhibiting or degrading the N-terminal domain of the androgen receptor, as well as methods for treating cancers such as prostate cancer.

### RELATED APPLICATIONS

This application claims the benefit of priority to U.S. Provisional Patent Application Ser. No. 62/826,636, filed Mar. 29, 2019. The contents of that application are hereby incorporated by reference in their entirety.

### STATEMENT REGARDING FEDERALLY SPONSORED RESEARCH

This invention was made with government support under Grant Numbers CA092131 and CA164331, awarded by the National Institutes of Health. The government has certain rights in the invention. This work was supported by the U.S. Department of Veterans Affairs, and the Federal Government has certain rights in the invention.

### BACKGROUND OF THE INVENTION

Prostate cancer is the most common cancer and the second leading cause of cancer death in Western men. When the cancer is confined locally, the disease can usually be treated by surgery or radiation. However, 30% of prostate cancers treated that way relapse with distant metastatic disease, and some patients have advanced disease at diagnosis. Advanced disease is treated by castration and/or administration of antiandrogens, the so-called androgen deprivation therapy. Castration lowers the circulating levels of androgens and reduces the activity of androgen receptor (AR). Administration of antiandrogens blocks AR function by competing away androgen binding, thereby reducing the AR activity. Although initially effective, these treatments quickly fail and the cancer becomes hormone refractory, or castration resistant.

Castration resistant prostate cancer (CRPC) is typified by persistent expression and transcriptional activity of the androgen receptor (AR). Over the last decade, pre-clinical models, correlative studies involving patient material, and clinical studies have provided the evidence to support the notion that inhibiting the AR represents a viable approach to effectively treat CRPC. Accordingly, improved inhibitors of the AR are needed.

### SUMMARY OF THE INVENTION

In certain aspects, the present invention provides compounds having the structure of formula I, II, III, IV, V, VI, VII, or VIII, or a pharmaceutically acceptable salt thereof:

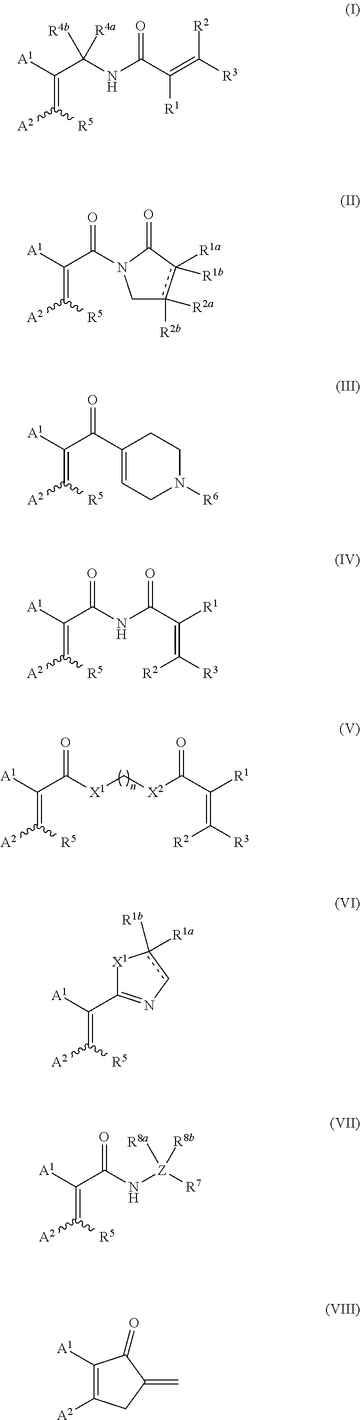

wherein:
A¹ is aryl or hetaryl;
A² is aryl or hetaryl;
R⁵ is H, alkyl, or halo;
R¹ is H, alkyl, haloalkyl, aralkyl, or hetaralkyl;
R² is H, alkyl, or haloalkyl;
R³ is H, alkyl, haloalkyl, aryl, or hetaryl;
R^{4a} and R^{4b} are each independently H or alkyl, or R^{4a} and R^{4b} combine to form oxo;
[symbol] is a single bond or a double bond,
when [symbol] is a single bond in Formula (II), R^{1a}, R^{1b}, R^{2a}, and R^{2b} are each independently H, alkyl, or alkoxy;
   when [symbol] is a double bond in Formula (II),
      R^{1a} and R^{2a} are each independently H, alkyl, or alkoxy, and
      R^{1b} and R^{2b} are absent;
when [symbol] is a single bond in Formula (VI), R^{1a} and R^{1b} combine to form CH₂;
when [symbol] is a double bond in Formula (VI), R^{1a} is H or alkyl and R^{1b} is absent;
R⁶ is H, alkyl, aralkyl, or hetaralkyl;
X¹ and X² are each independently NH or O;
n is 1-4;
X is O, NH, or S;
R⁷ is amino, alkynyl, cyano, cycloalkyl, alkyl, or alkenyl;
Z is S or C;
   when Z is S, R^{8a} and R^{8b} are each oxo;
   when Z is C,
   R^{8a} and R^{8b} are each independently H or alkyl, or
   R^{8a} and R^{8b} combine to form oxo, or
R^{8a} and R^{8b} combine to form a cyclopropyl ring including Z.

In certain preferred embodiments, when A¹ and A² are both phenyl in Formula (VIII), at least one of A¹ and A² is substituted.

In certain preferred embodiments, the compound of Formula I, II, III, IV, V, VI, VII, or VIII is not:

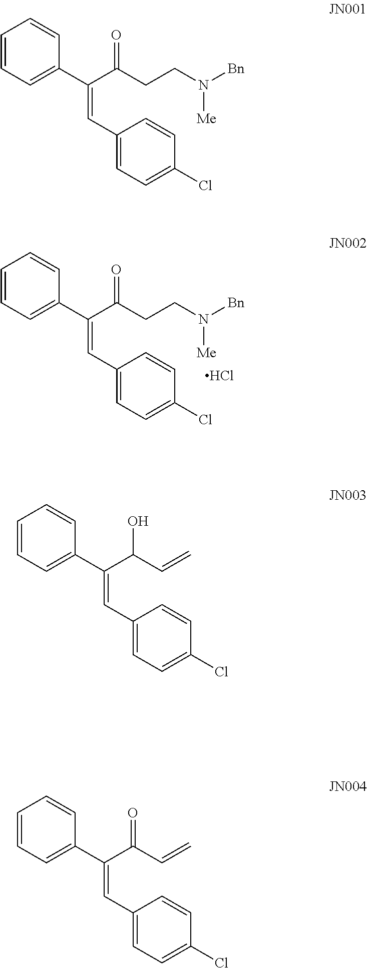 [SMILES: CN(CCC(=O)/C(=C\c1ccc(Cl)cc1)c1ccccc1)Cc1ccccc1 ; CN(CCC(=O)/C(=C\c1ccc(Cl)cc1)c1ccccc1)Cc1ccccc1 ; Cl ; C=CC(O)/C(=C\c1ccc(Cl)cc1)c1ccccc1 ; C=CC(=O)/C(=C\c1ccc(Cl)cc1)c1ccccc1]

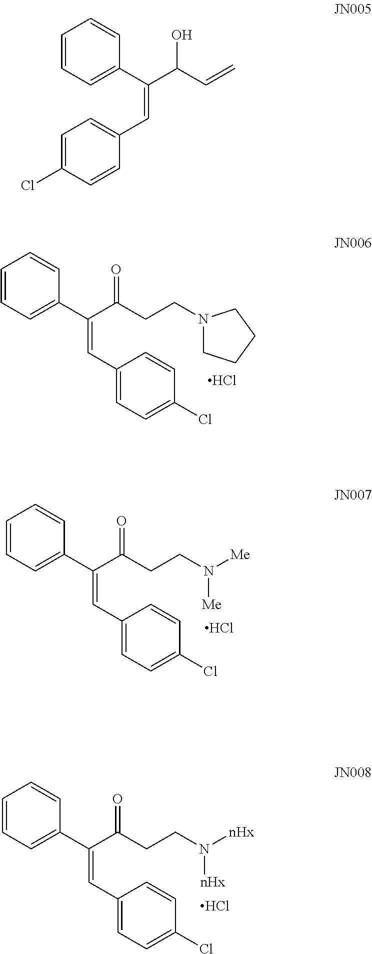

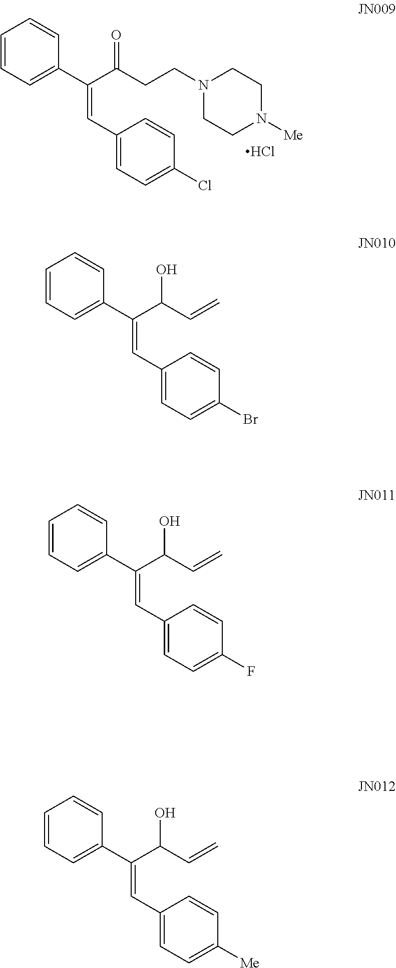 [SMILES: CN1CCN(CCC(=O)/C(=C\c2ccc(Cl)cc2)c2ccccc2)CC1 ; Cl ; C=CC(O)/C(=C\c1ccc(Br)cc1)c1ccccc1 ; C=CC(O)/C(=C\c1ccc(F)cc1)c1ccccc1 ; C=CC(O)/C(=C\c1ccc(C)cc1)c1ccccc1]

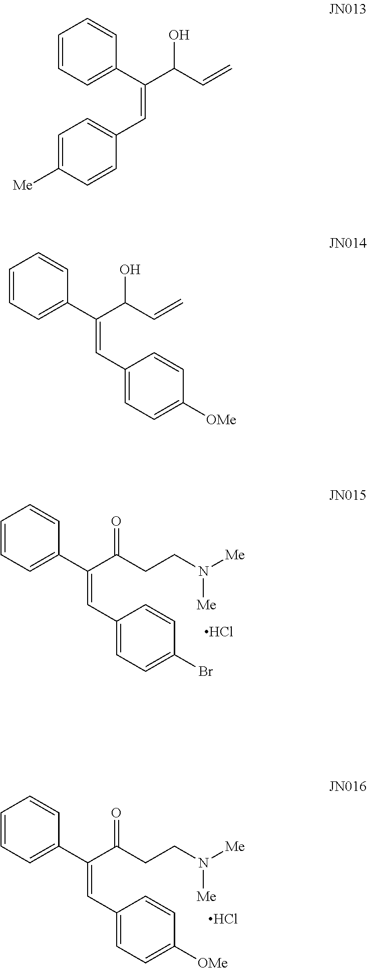 [SMILES: C=CC(O)/C(=C/c1ccc(C)cc1)c1ccccc1 ; C=CC(O)/C(=C\c1ccc(OC)cc1)c1ccccc1 ; CN(C)CCC(=O)/C(=C\c1ccc(Br)cc1)c1ccccc1 ; Cl ; COc1ccc(/C=C(\C(=O)CCN(C)C)c2ccccc2)cc1 ; Cl]

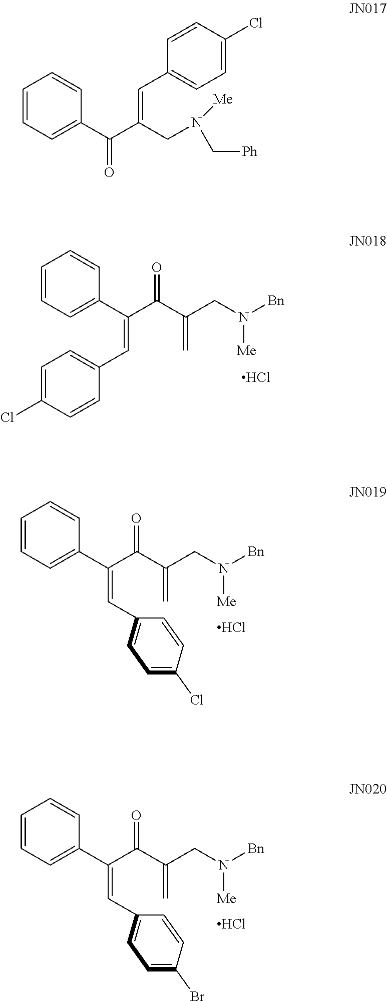

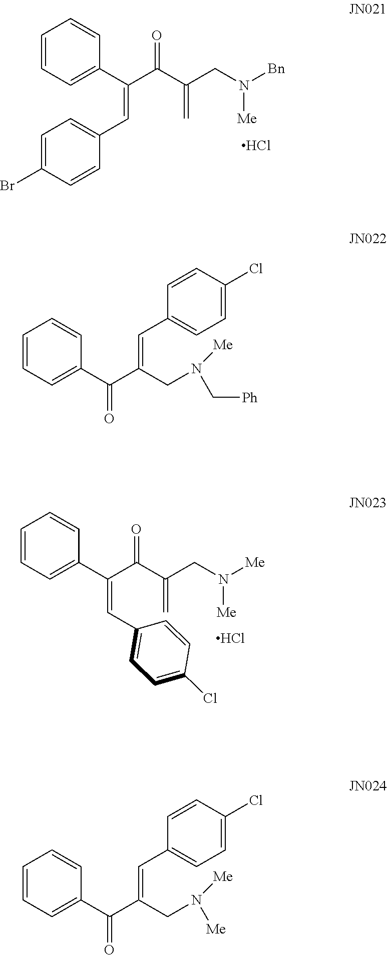

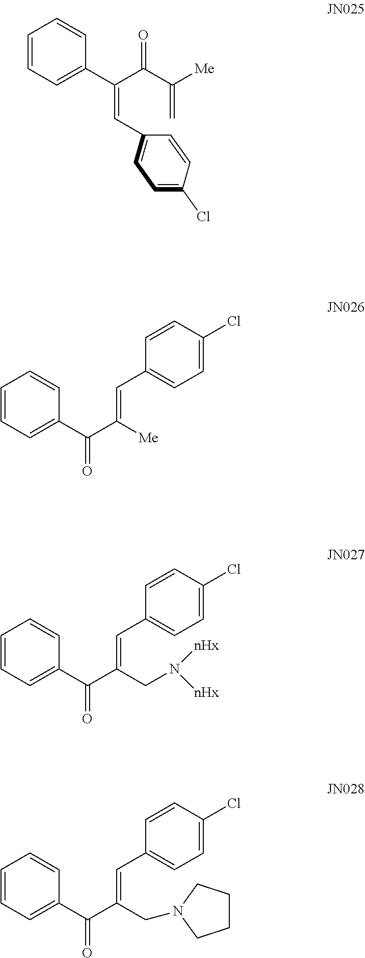

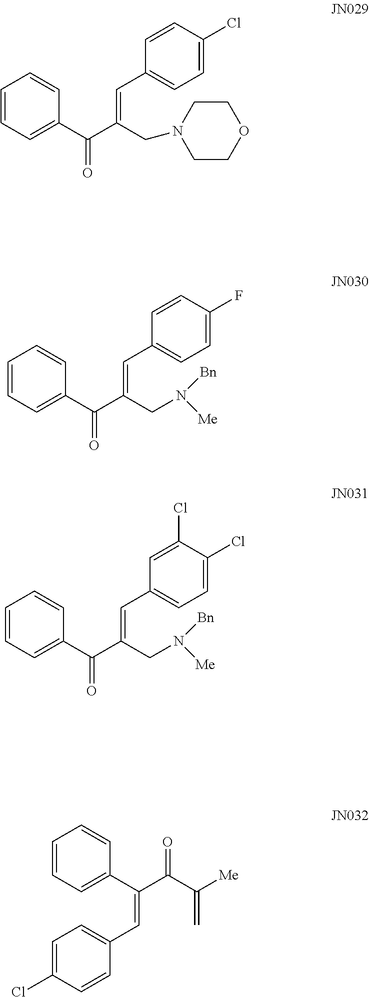 [SMILES: O=C(/C(=C/c1ccc(Cl)cc1)CN1CCOCC1)c1ccccc1 ; CN(C/C(=C\c1ccc(F)cc1)C(=O)c1ccccc1)Cc1ccccc1 ; CN(C/C(=C\c1ccc(Cl)c(Cl)c1)C(=O)c1ccccc1)Cc1ccccc1 ; C=C(C)C(=O)/C(=C/c1ccc(Cl)cc1)c1ccccc1]

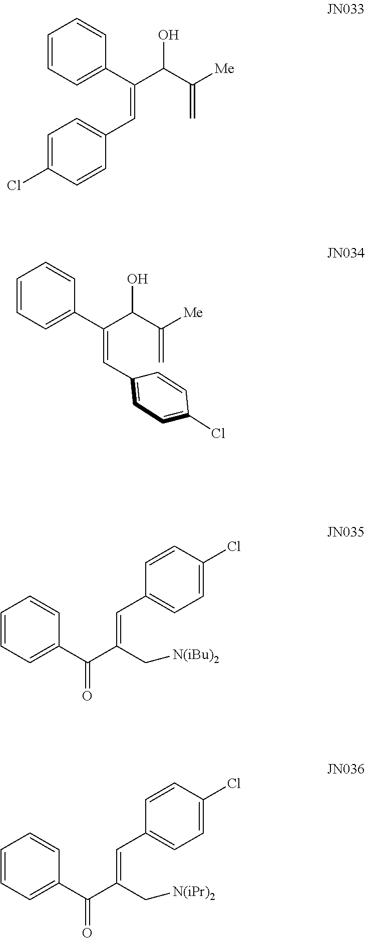

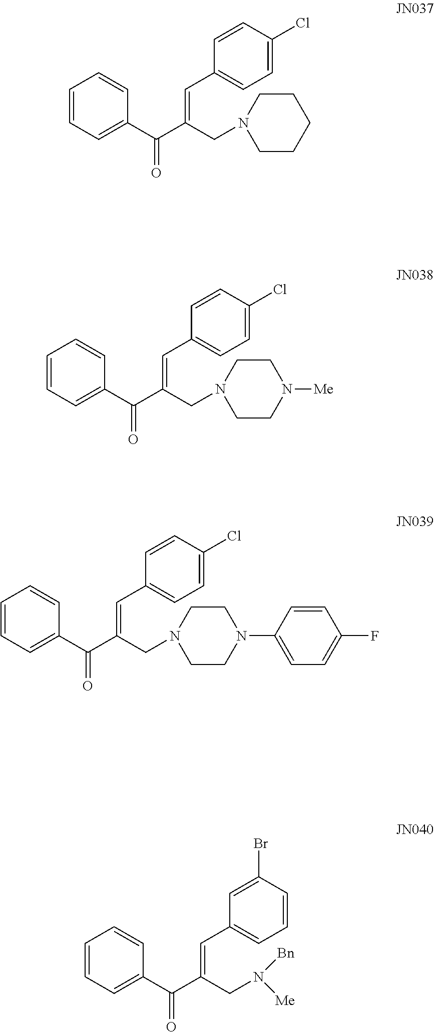 [SMILES: O=C(/C(=C/c1ccc(Cl)cc1)CN1CCCCC1)c1ccccc1 ; CN1CCN(C/C(=C\c2ccc(Cl)cc2)C(=O)c2ccccc2)CC1 ; O=C(/C(=C/c1ccc(Cl)cc1)CN1CCN(c2ccc(F)cc2)CC1)c1ccccc1 ; CN(C/C(=C\c1cccc(Br)c1)C(=O)c1ccccc1)Cc1ccccc1]

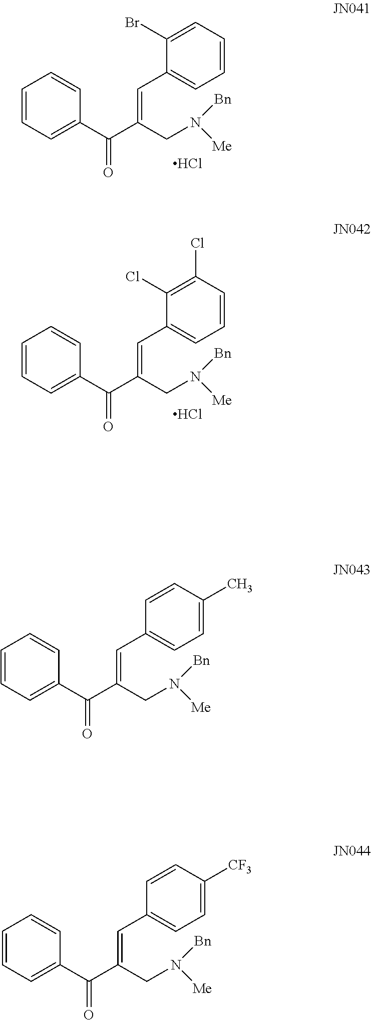 [SMILES: CN(C/C(=C\c1ccccc1Br)C(=O)c1ccccc1)Cc1ccccc1 ; Cl ; CN(C/C(=C\c1cccc(Cl)c1Cl)C(=O)c1ccccc1)Cc1ccccc1 ; Cl ; Cc1ccc(/C=C(\CN(C)Cc2ccccc2)C(=O)c2ccccc2)cc1 ; CN(C/C(=C\c1ccc(C(F)(F)F)cc1)C(=O)c1ccccc1)Cc1ccccc1]

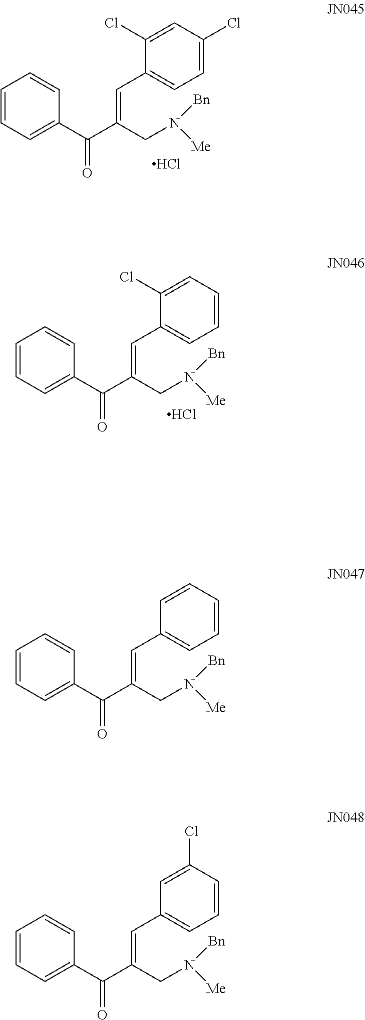 [SMILES: CN(C/C(=C\c1ccc(Cl)cc1Cl)C(=O)c1ccccc1)Cc1ccccc1 ; Cl ; CN(C/C(=C\c1ccccc1Cl)C(=O)c1ccccc1)Cc1ccccc1 ; Cl ; CN(C/C(=C\c1ccccc1)C(=O)c1ccccc1)Cc1ccccc1 ; CN(C/C(=C\c1cccc(Cl)c1)C(=O)c1ccccc1)Cc1ccccc1]

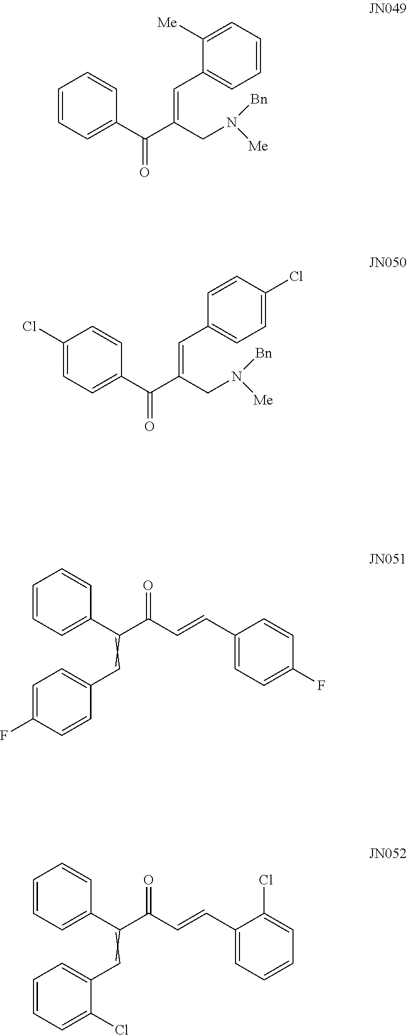 [SMILES: Cc1ccccc1/C=C(\CN(C)Cc1ccccc1)C(=O)c1ccccc1 ; CN(C/C(=C\c1ccc(Cl)cc1)C(=O)c1ccc(Cl)cc1)Cc1ccccc1 ; O=C(/C=C/c1ccc(F)cc1)C(=Cc1ccc(F)cc1)c1ccccc1 ; O=C(/C=C/c1ccccc1Cl)C(=Cc1ccccc1Cl)c1ccccc1]

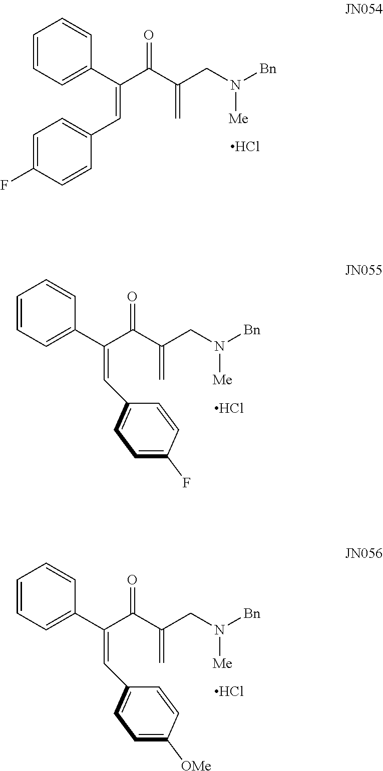

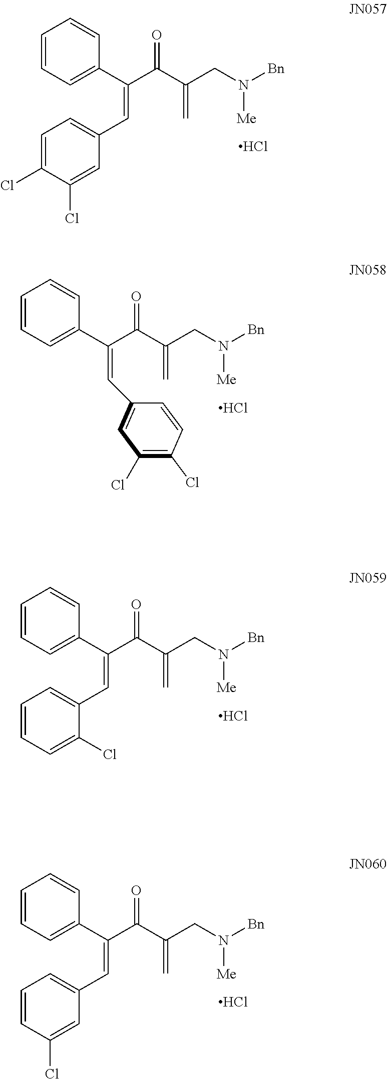

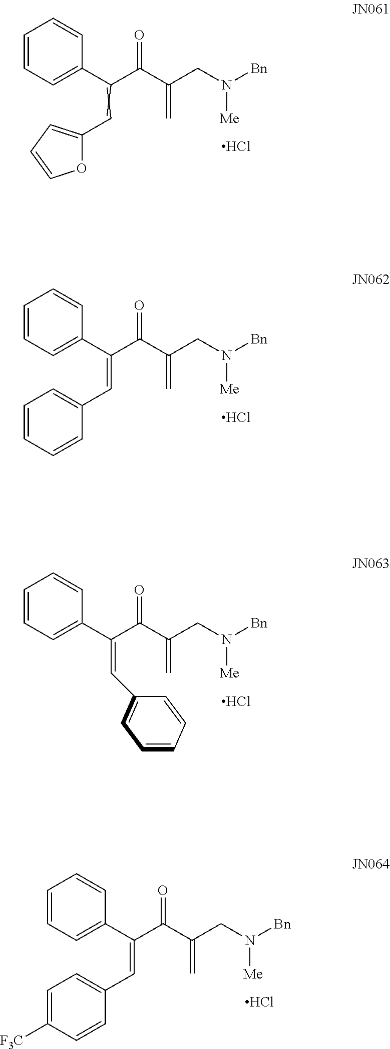

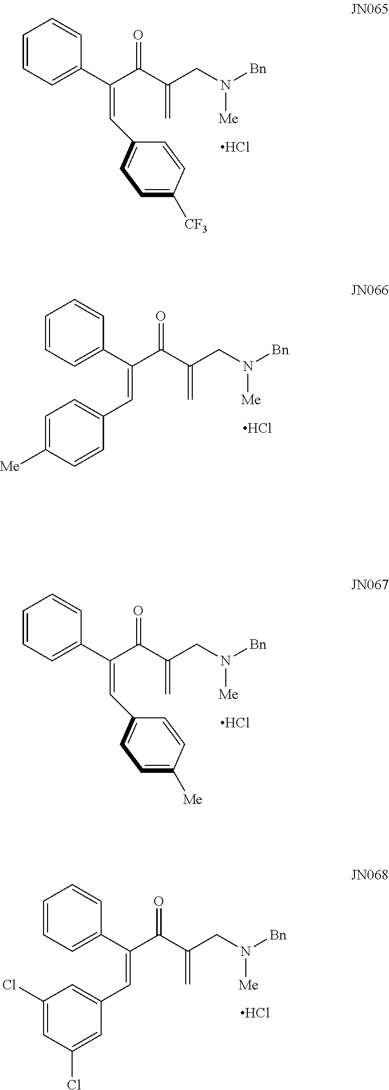

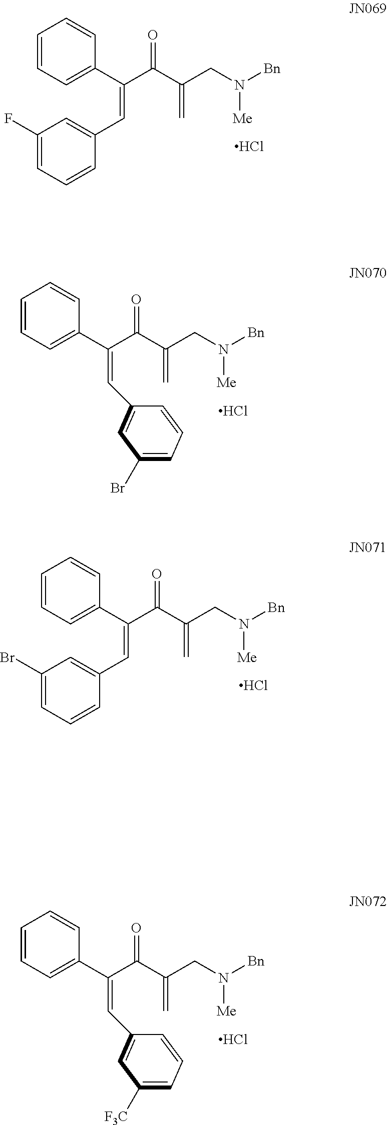

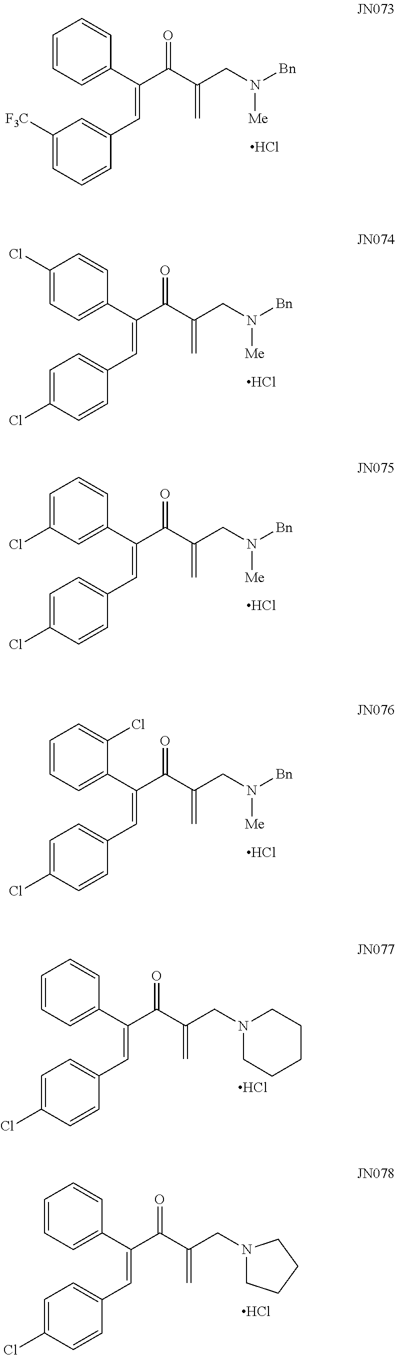

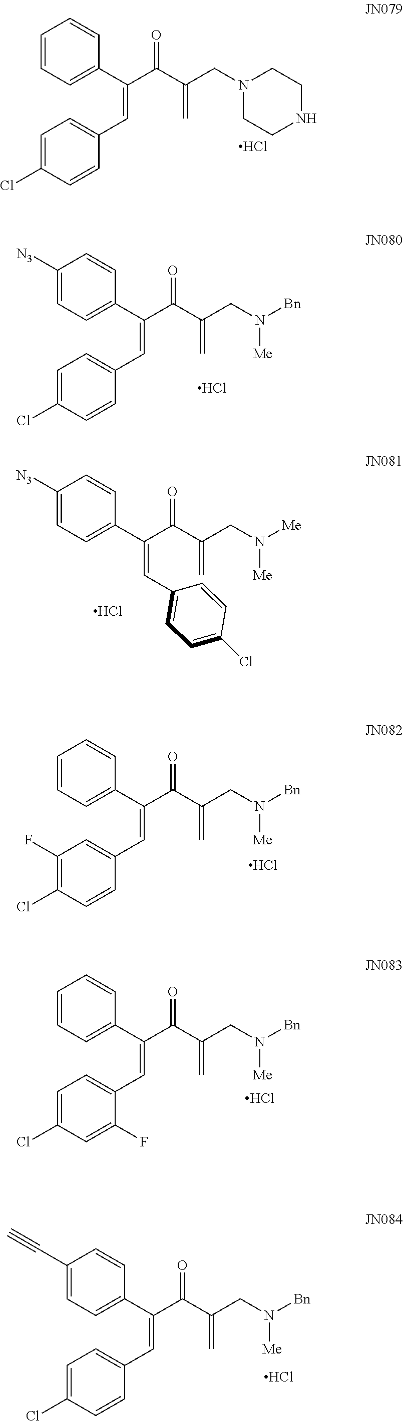

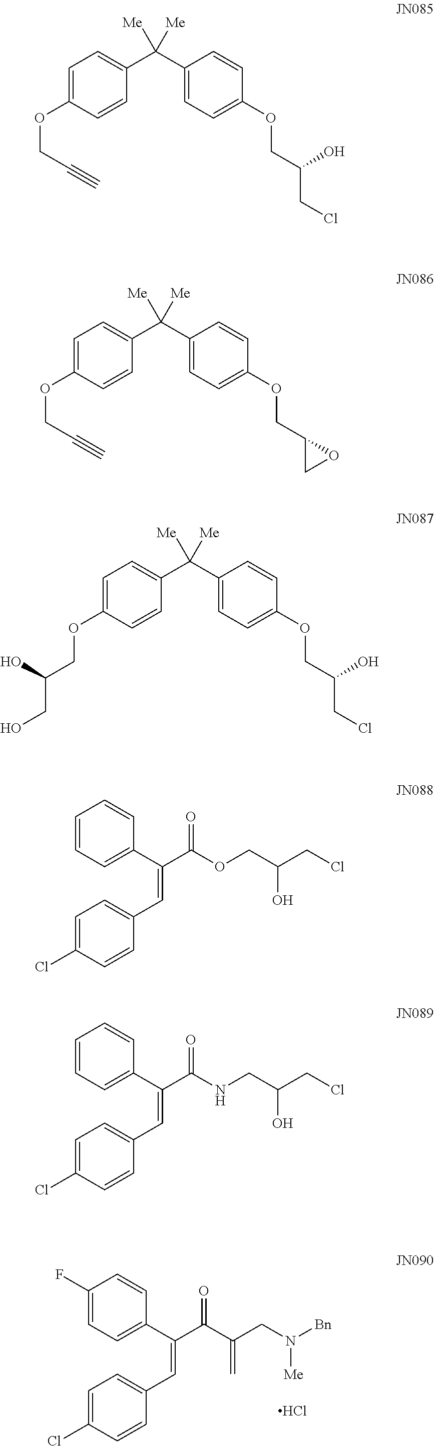

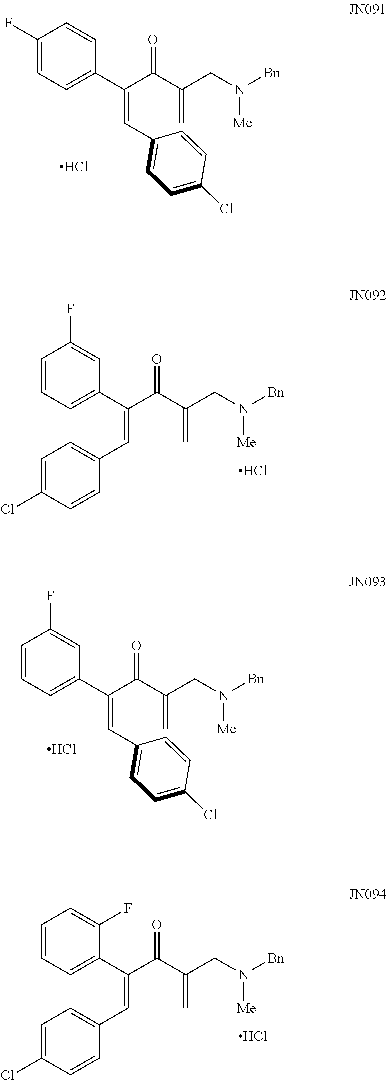

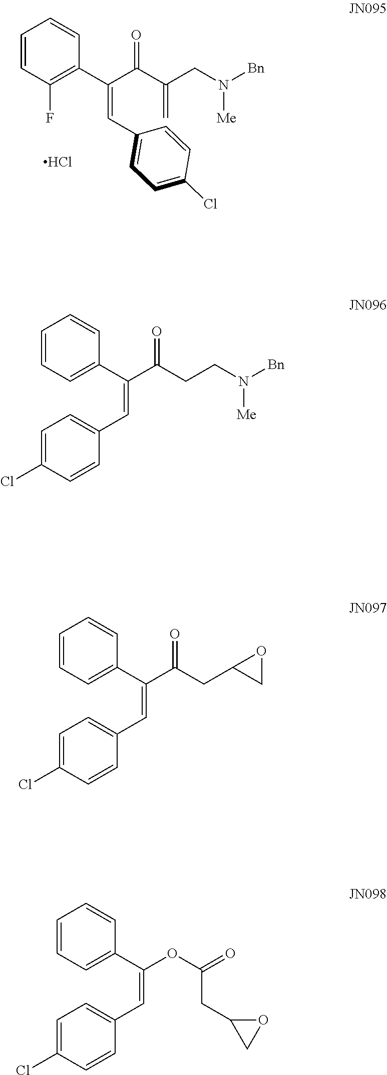

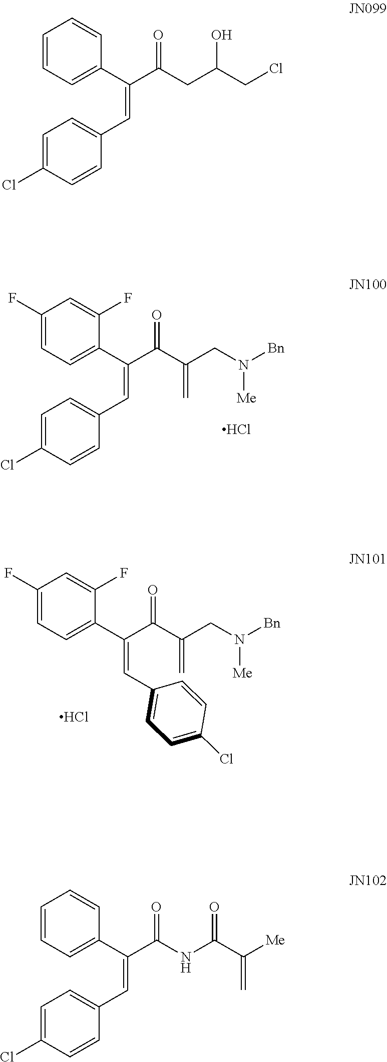

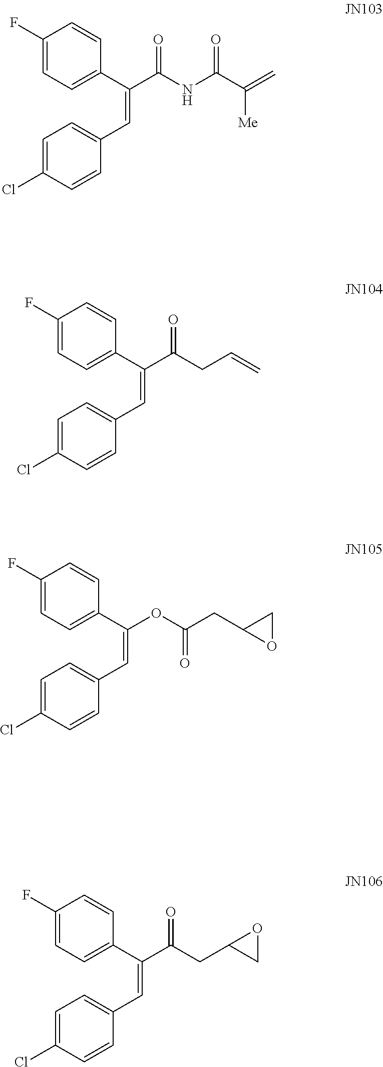 [SMILES: C=C(C)C(=O)NC(=O)/C(=C/c1ccc(Cl)cc1)c1ccc(F)cc1 ; C=CCC(=O)/C(=C/c1ccc(Cl)cc1)c1ccc(F)cc1 ; O=C(CC1CO1)O/C(=C/c1ccc(Cl)cc1)c1ccc(F)cc1 ; O=C(CC1CO1)/C(=C/c1ccc(Cl)cc1)c1ccc(F)cc1]

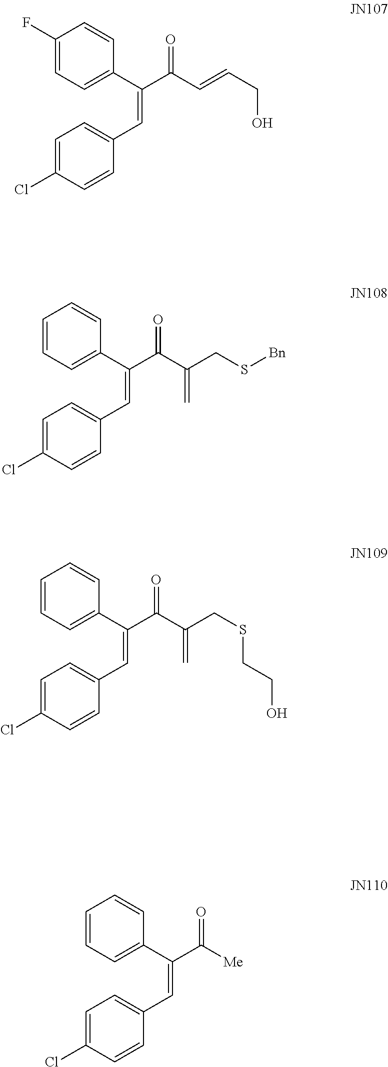 [SMILES: O=C(/C=C/CO)/C(=C/c1ccc(Cl)cc1)c1ccc(F)cc1 ; C=C(CSCc1ccccc1)C(=O)/C(=C/c1ccc(Cl)cc1)c1ccccc1 ; C=C(CSCCO)C(=O)/C(=C/c1ccc(Cl)cc1)c1ccccc1 ; CC(=O)/C(=C/c1ccc(Cl)cc1)c1ccccc1]

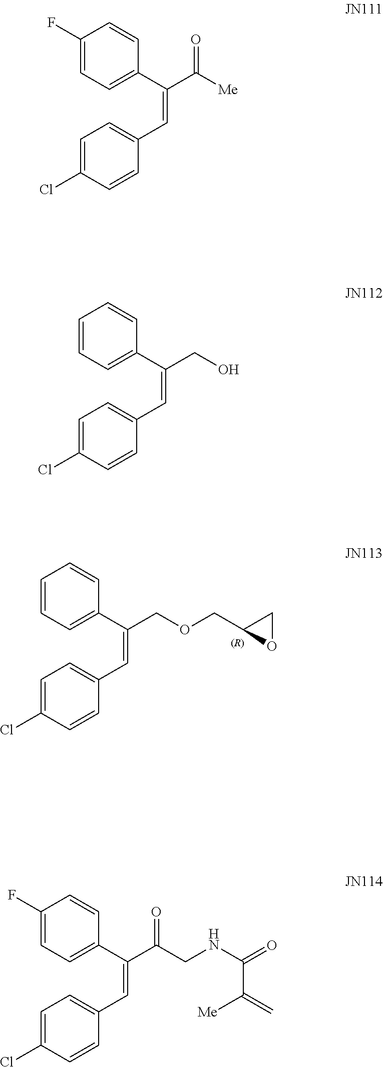

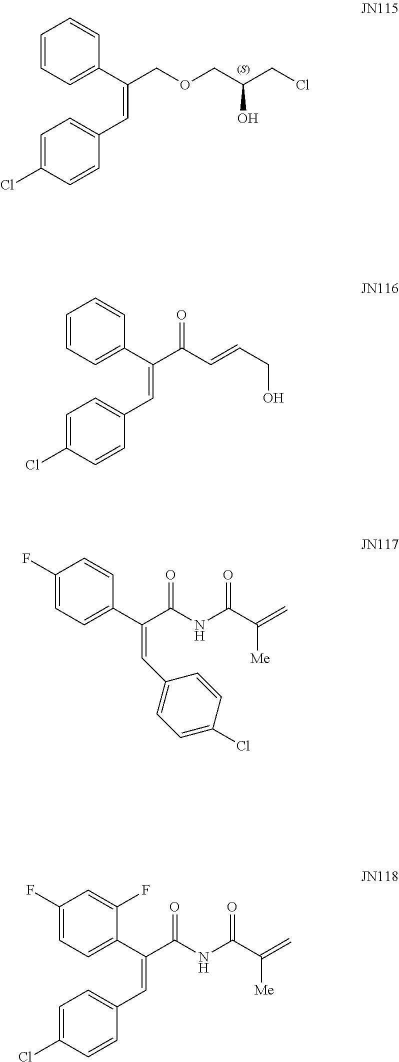

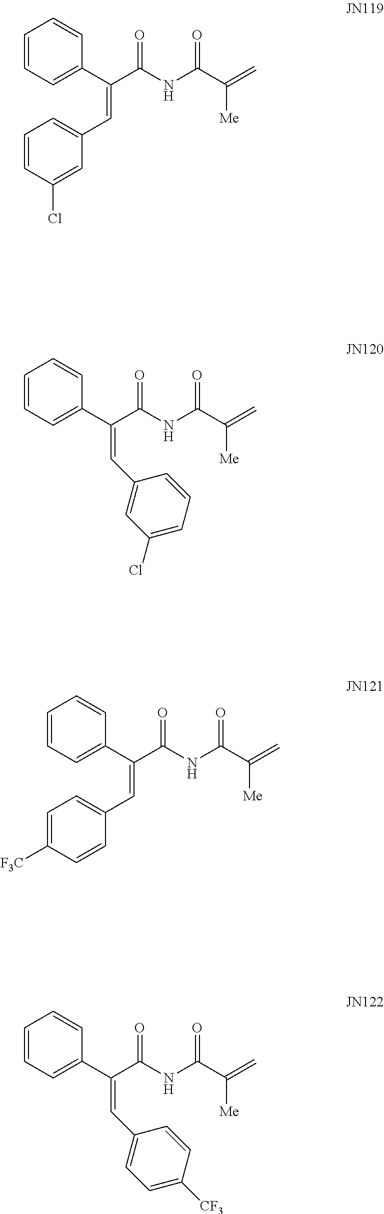 [SMILES: C=C(C)C(=O)NC(=O)/C(=C/c1cccc(Cl)c1)c1ccccc1 ; C=C(C)C(=O)NC(=O)/C(=C\c1cccc(Cl)c1)c1ccccc1 ; C=C(C)C(=O)NC(=O)/C(=C/c1ccc(C(F)(F)F)cc1)c1ccccc1 ; C=C(C)C(=O)NC(=O)/C(=C\c1ccc(C(F)(F)F)cc1)c1ccccc1]

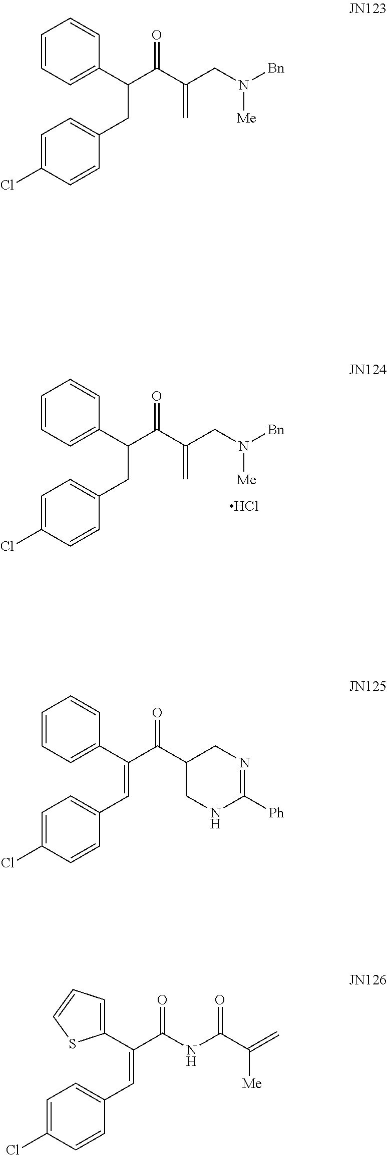 [SMILES: C=C(CN(C)Cc1ccccc1)C(=O)C(Cc1ccc(Cl)cc1)c1ccccc1 ; C=C(CN(C)Cc1ccccc1)C(=O)C(Cc1ccc(Cl)cc1)c1ccccc1 ; Cl ; O=C(/C(=C/c1ccc(Cl)cc1)c1ccccc1)C1CN=C(c2ccccc2)NC1 ; C=C(C)C(=O)NC(=O)/C(=C/c1ccc(Cl)cc1)c1cccs1]

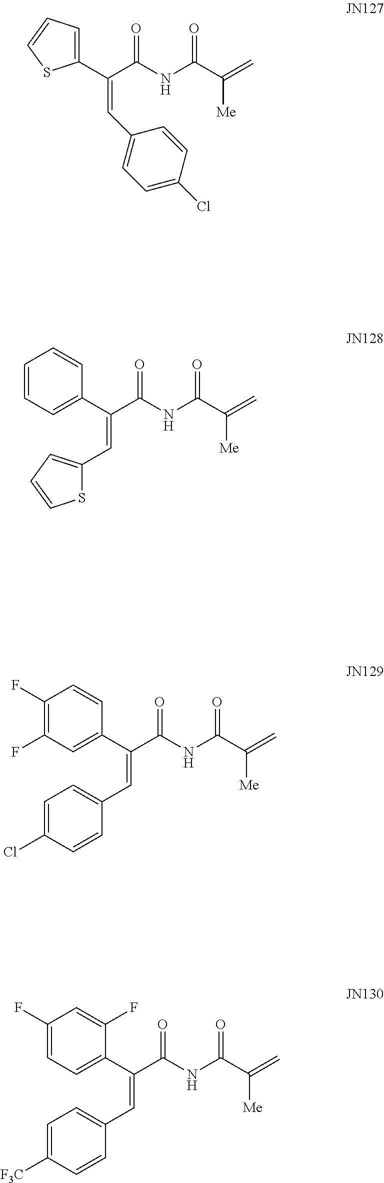 [SMILES: C=C(C)C(=O)NC(=O)/C(=C\c1ccc(Cl)cc1)c1cccs1 ; C=C(C)C(=O)NC(=O)/C(=C/c1cccs1)c1ccccc1 ; C=C(C)C(=O)NC(=O)/C(=C/c1ccc(Cl)cc1)c1ccc(F)c(F)c1 ; C=C(C)C(=O)NC(=O)/C(=C/c1ccc(C(F)(F)F)cc1)c1ccc(F)cc1F]

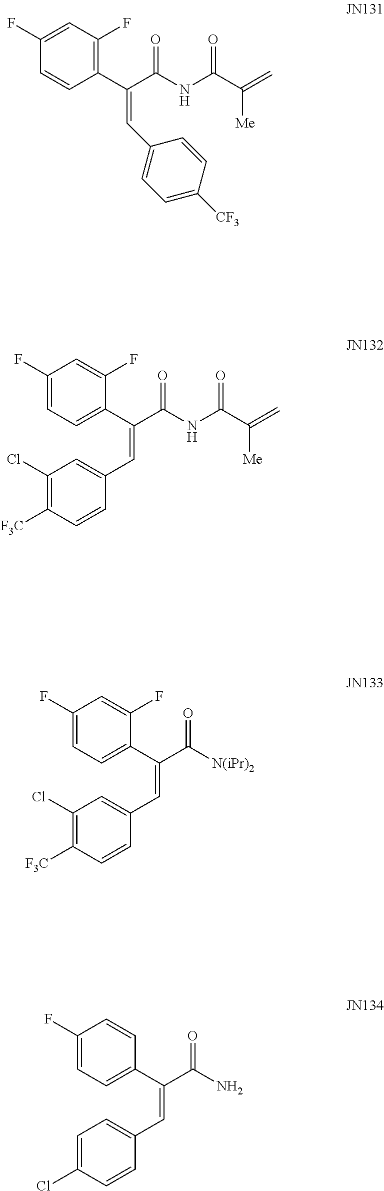

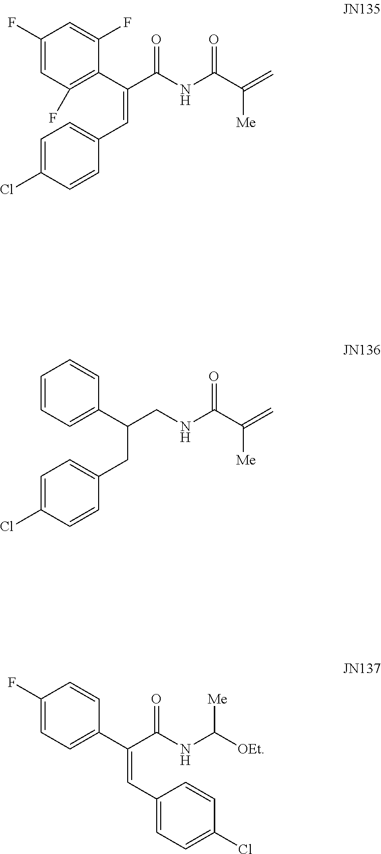 [SMILES: C=C(C)C(=O)NC(=O)/C(=C/c1ccc(Cl)cc1)c1c(F)cc(F)cc1F ; C=C(C)C(=O)NCC(Cc1ccc(Cl)cc1)c1ccccc1 ; CCOC(C)NC(=O)/C(=C\c1ccc(Cl)cc1)c1ccc(F)cc1]

Exemplary compounds of Formulas I, II, III, IV, V, VI, VII, and VIII include the compounds depicted in Table I.

In certain aspects, the present invention provides a solid form of compound JN032

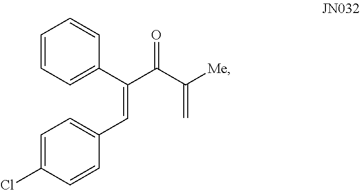 [SMILES: C=C(C)C(=O)/C(=C/c1ccc(Cl)cc1)c1ccccc1]

characterized by X-ray powder diffraction peaks at 2θ angles of about 21.5°, about 22.6°, and about 27.3°.

In certain aspects, the present invention provides a solid form, which is Form I of compound JN110

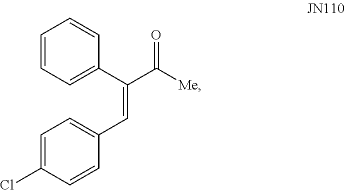 [SMILES: CC(=O)/C(=C/c1ccc(Cl)cc1)c1ccccc1]

characterized by X-ray powder diffraction peaks at 2θ angles of about 17.6°, about 22.2°, and about 28.8°.

In certain aspects, the present invention provides a solid form, which is Form I of compound JN034

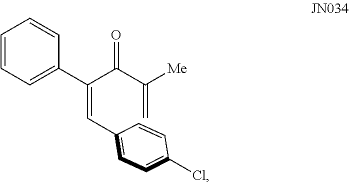

characterized by X-ray powder diffraction peaks at 2θ angles of about 8.3°, about 17.7°, and about 22.4°.

In certain aspects, the present invention provides a solid form, which is Form I of compound JN097

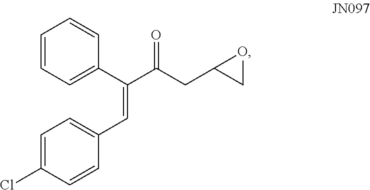 [SMILES: O=C(CC1CO1)/C(=C/c1ccc(Cl)cc1)c1ccccc1]

characterized by X-ray powder diffraction peaks at 2θ angles of about 20.5°, about 23.1°, and about 27.0°.

In certain aspects, the present invention provides a solid form, which is Form I of compound JN117

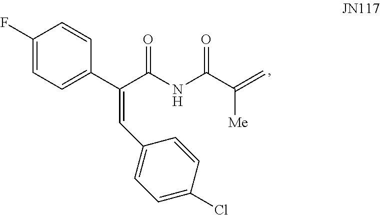 [SMILES: C=C(C)C(=O)NC(=O)/C(=C\c1ccc(Cl)cc1)c1ccc(F)cc1]

characterized by X-ray powder diffraction peaks at 2θ angles of about 7.8°, about 16.4°, and about 21.5°.

In certain aspects, the present invention provides a solid form, which is Form I of compound JN103

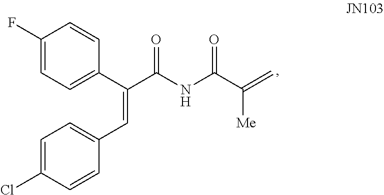 [SMILES: C=C(C)C(=O)NC(=O)/C(=C/c1ccc(Cl)cc1)c1ccc(F)cc1]

characterized by X-ray powder diffraction peaks at 2θ angles of about 6.6°, about 18.0°, and about 21.6°.

The invention further relates to pharmaceutical compositions of the subject compounds, as well as methods of using these compounds or compositions in the treatment of cancer, such as prostate cancer.

### BRIEF DESCRIPTION OF THE DRAWINGS

FIG. 1 is a schematic depiction of cellular processes related to AR signaling and therapeutic targeting. A) Physiologic regulation of androgen synthesis. Pulsatile secretion of LHRH induces luteinizing hormone (LH) secretion by the anterior pituitary, which in turn drives testosterone (T) synthesis and secretion by the testes, from which 90-95% of androgens are derived. LHRH analogues, by providing continuous, unremitting engagement of the LHRH receptors on the anterior pituitary, suppress LH secretion. The adrenal glands are a minor source of androgens; adrenal androgens (e.g. DHEA) are converted into T or dihydrotestosterone (DHT) in peripheral tissues. B) AR working mechanism. Upon ligand binding, AR dimerizes, translocates to the nucleus, and induces gene transcription. Novel AR targeting agents (in red), inhibit intratumoral steroidogenesis (e.g., abiraterone, a 17α-hydroxylase inhibitor) or function as pure AR antagonists (e.g., MDV3100). C) Schematic of full-length AR (AR^{FL}), the constitutively active ARΔLBD, and a Y1H system that can serve as the basis for a high-throughput screening assay. The ligand-independent ARΔLBD, when expressed in our genetically modified, drug permeable yeast strain, binds to tandem copies of the ARE, which induces the expression of a reporter gene. ⊥ inhibition; → activation; NLS: nuclear localization signal.

FIG. 2. Schematic of primary amino acid structure of full-length AR and a constitutively active AR splice variant that lacks a functional LBD.

FIGS. 3A-Q. Growth inhibitory effects of selected compounds. The indicated cells were exposed to the indicated compounds for 6 days; cell viability was measured by MTT assay, and specific reporters were assayed using literature conditions. Results were normalized to that of vehicle control. Experiments were performed in quadruplicate; results are means±s.d.

FIG. 3A. 22Rv1 cells. For each concentration in the figure, the bars represent relative cell viability for JN143, JN144, JN145, JN146, JN147, 3100-17, 3100-18, JN118, and JN121, from left to right.

FIG. 3B. 22Rv1 cells were exposed to the indicated compounds for 6 days; cell viability was measured by MTT assay. Results were normalized to that of vehicle control. Experiments were performed in quadruplicate; results are means±s.d. For each concentration in the figure, the bars represent relative cell viability for JN148, JN149, JN150, JN151, JN152, JN103, JN3100-724, JN3100-18, from left to right.

FIG. 3C. 22Rv1 cells (blue), LNCaP AR cells (red), and PC3 cells (green). For each concentration in the figure, the bars represent cell viability for JN148, JN149, JN150, JN151, JN152, JN103, JN3100-724, JN3100-18, from left to right.

FIG. 3D. 22Rv1 cells (red), LNCaP AR cells (blue), and PC3 cells (green). For each concentration in the figure, the bars represent cell viability for JN152, JN155, JN103, and JN154, from left to right.

FIG. 3E. 22Rv1 cells (brown), LNCaP AR cells (blue), and PC3 cells (green). For each concentration in the figure, the bars represent cell viability for JN138, JN139, JN140, JN141, JN142, JN103, from left to right.

FIG. 3F. LNCaP AR cells. For each concentration in the figure, the bars represent cell viability for JN143, JN144, JN145, JN146, JN147, 3100-17, 3100-18, JN118, and JN121, from left to right.

FIG. 3G. LNCaP AR cells. For each concentration in the figure, the bars represent cell viability for JN148, JN149, JN150, JN151, JN152, JN103, JN3100-724, JN1300-18, from left to right.

FIG. 3H. LNCaP AR cells. For each concentration in the figure, the bars represent cell viability for JN152, JN153, JN103, and JN154, from left to right.

FIG. 3I. LNCaP AR cells. For each concentration in the figure, the bars represent MMTV reporter assay data for JN152 and JN103, from left to right.

FIG. 3J. For each concentration in the figure, the bars represent MMTV reporter assay data in LNCaP AR cells (brown), Gal4-AR reporter assay data in PC3 cells (blue), GRE reporter assay data in PC3 cells (yellow), and CREB-reporter assay data in PC3 cells (green) for JN152 and JN103, from left to right.

FIG. 3K. LNCaP AR cells (brown), 22Rv1 cells (blue), and PC3 cells (green). For each concentration in the figure, the bars represent cell viability data for JN153, JN154, JN155, JN156, and JN103, from left to right.

FIG. 3L. PC3 cells. For each concentration in the figure, the bars represent luciferase reporter assay data for JN152 and JN103, from left to right.

FIG. 3M. PC3 cells. For each concentration in the figure, the bars represent Gal4-AR reporter assay data for JN152 and JN103, from left to right.

FIG. 3N. PC3 cells. For each concentration in the figure, the bars represent GRE reporter assay data for JN152 and JN103, from left to right.

FIG. 3O. PC3 cells. For each concentration in the figure, the bars represent cell viability data for JN143, JN144, JN145, JN146, JN147, 3100-17, 3100-18, JN118, and JN121, from left to right.

FIG. 3P. PC3 cells. For each concentration in the figure, the bars represent cell viability data for JN148, JN149, JN150, JN151, JN152, JN103, JN3100-724, and JN3100-18, from left to right.

FIG. 3Q. PC3 cells. For each concentration in the figure, the bars represent cell viability data for JN152, JN155, JN103, and JN154, from left to right.

FIG. 4 shows an x-ray powder diffraction (XRPD) spectrum for compound JN032.

FIG. 5 shows an XRPD spectrum for compound JN110.

FIG. 6 shows an XRPD spectrum for compound JN034.

FIG. 7 shows an XRPD spectrum for compound JN097.

FIG. 8 shows an XRPD spectrum for compound JN117.

FIG. 9 shows an XRPD spectrum for compound JN103.

FIG. 10 shows gene set expression analysis of 22Rv1 and LNCaP-AR cells treated with JN103 (10 μM) for 8 hours. Negative enrichment scores (NES are shown for the AR transcriptional program.

FIG. 11A shows selective degradation of LNCaP-AR cells by JN103.

FIG. 11B shows selective degradation of LNCaP-95 cells by JN103.

FIG. 11C shows selective degradation of HEK-293 cells that were engineered to ectopically express ARΔ567 by JN103.

FIG. 11D shows selective degradation of PC3 cells by JN103.

FIG. 11E shows selective degradation of T47D breast cancer cells by JN103.

FIG. 12 shows colony formation assays of DU145, PC3LNCaP-AR (full-length AR), 22Rv1 (full-length and splice variant AR), and VCaP cells, which were treated with JN103.

FIG. 13 shows growth-inhibitory effects of JN103 in MTT assays on 20 non-prostate cancer cell lines.

### DETAILED DESCRIPTION

In certain aspects, the present disclosure provides compounds having the structure of formula I, II, III, IV, V, VI, VII, or VIII, and pharmaceutically acceptable salts thereof:

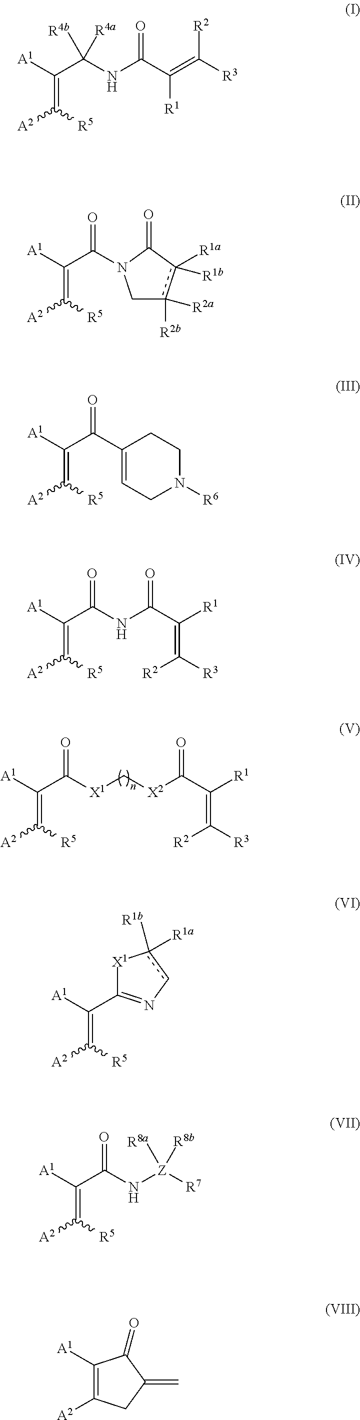

wherein:
A¹ is aryl or hetaryl;
A² is aryl or hetaryl;
R⁵ is H, alkyl, or halo;
R¹ is H, alkyl, haloalkyl, aralkyl, or hetaralkyl;
R² is H, alkyl, or haloalkyl;
R³ is H, alkyl, haloalkyl, aryl, or hetaryl;
R^{4a} and R^{4b} are each independently H or alkyl, or R^{4a} and R^{4b} combine to form oxo;
[symbol] is a single bond or a double bond,
when [symbol] is a single bond in Formula (II), R₁ₐ, R^{1b}, R^{2a}, and R^{2b} are each independently H, alkyl, or alkoxy;
   when [symbol] is a double bond in Formula (II),
      R^{1a} and R^{2a} are each independently H, alkyl, or alkoxy, and
      R^{1b} and R^{2b} are absent;
when [symbol] is a single bond in Formula (VI), R^{1a} and R^{1b} combine to form CH₂;
when [symbol] is a double bond in Formula (VI), R^{1a} is H or alkyl and R^{1b} is absent;
R⁶ is H, alkyl, aralkyl, or hetaralkyl;
X¹ and X² are each independently NH or O;
n is 1-4;
X is O, NH, or S;
R⁷ is amino, alkynyl, cyano, cycloalkyl, alkyl, or alkenyl;
Z is S or C;
   when Z is S, R^{8a} and R^{8b} are each oxo;
   when Z is C,
   R^{8a} and R^{8b} are each independently H or alkyl, or
   R^{8a} and R^{8b} combine to form oxo, or
   R^{8a} and R^{8b} combine to form a cyclopropyl ring including Z.

In certain embodiments, the disclosure provides compounds of formula VIII, wherein when A¹ and A² are both phenyl, at least one of A¹ and A² is substituted.

In certain embodiments, the disclosure provides compounds having the structure of formula (Ia), (Ib), (IIa), (IIb), (IIc), (Va), (Vb), (VIa), (VIb), (VIIa), (VIIb), or (VIII):

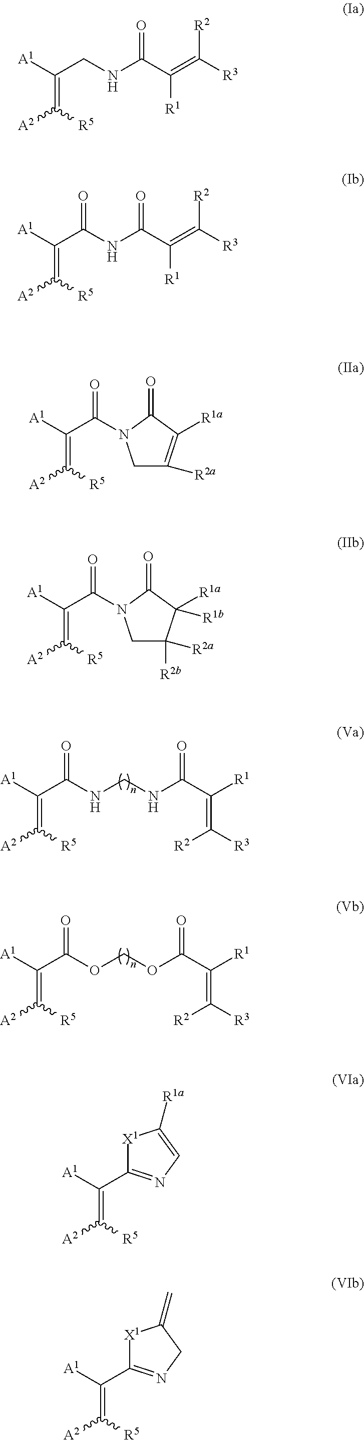

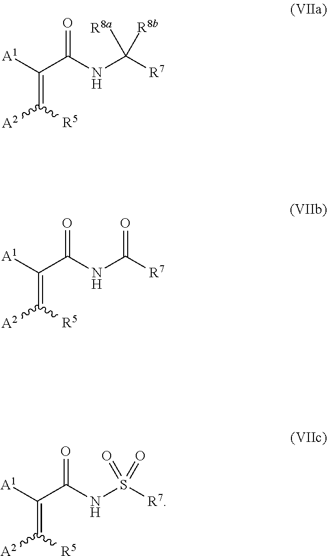

In certain embodiments, the compound is represented by formula I., such as formula Ia or formula Ib. In certain embodiments, the compound is represented by formula II, such as formula IIa or formula IIb. In certain embodiments, the compound is represented by formula III. In certain embodiments, the compound is represented by formula IV. In certain embodiments, the compound is represented by formula V, such as formula Va or formula Vb. In certain embodiments, the compound is represented by formula VI, such as formula VIa or formula VIb. In certain embodiments, the compound is represented by formula VII, such as formula VIIa, VIIb, or VIIc. In certain embodiments, the compound is represented by formula VIII.

In certain preferred embodiments of the formulas described herein, A¹ and A² are cis to each other.

In certain embodiments, A² is aryl unsubstituted or substituted with one or more R¹¹, wherein each R¹¹ is independently selected from halo, alkyl, haloalkyl, hydroxyl, cyano, alkoxy, alkynyl, or azido. In certain such embodiments, A² is chlorophenyl.

In certain other embodiments, A² is heteroaryl unsubstituted or substituted with one or more R¹¹, wherein each R¹¹ is independently selected from halo, alkyl, haloalkyl, hydroxyl, cyano, alkoxy, alkynyl, or azido. In certain such embodiments, A² is pyridyl (e.g. pyrid-3-yl) substituted with trifluoromethyl, such as 5-trifluoromethyl pyrid-3-yl.

In certain embodiments, A¹ is phenyl.

In certain embodiments, A¹ is unsubstituted.

In certain embodiments, A¹ is unsubstituted or substituted with at least one R¹² wherein each R¹² is independently selected from halo, alkyl, haloalkyl, hydroxyl, cyano, alkoxy, alkynyl, or azido. In certain such embodiments, A¹ is substituted by at least one R¹².

In certain embodiments, wherein R⁵ is H or alkyl. In certain such embodiments, R⁵ is H.

In certain embodiments, R¹ is H or methyl.

In certain embodiments, R² is H.

In certain embodiments, R³ is H, haloalkyl, or aryl.

In certain embodiments, R^{4a} and R^{4b} are each H. In certain other embodiments, R^{4a} and R^{4b} combine to form an oxo.

In certain embodiments of formula III, R⁶ is aryl. In certain embodiments, R⁶ is benzyl.

In certain embodiments of formula IV, R³ is H, haloalkyl, or aryl, such as H, trifluoromethyl, or phenyl. In certain further embodiments, R¹ is H, methyl, or benzyl. In certain embodiments of formula IV, such as when R³ is H, haloalkyl, or aryl, R¹ and R² are trans to each other.

In certain embodiments, the present disclosure provides compounds selected from:

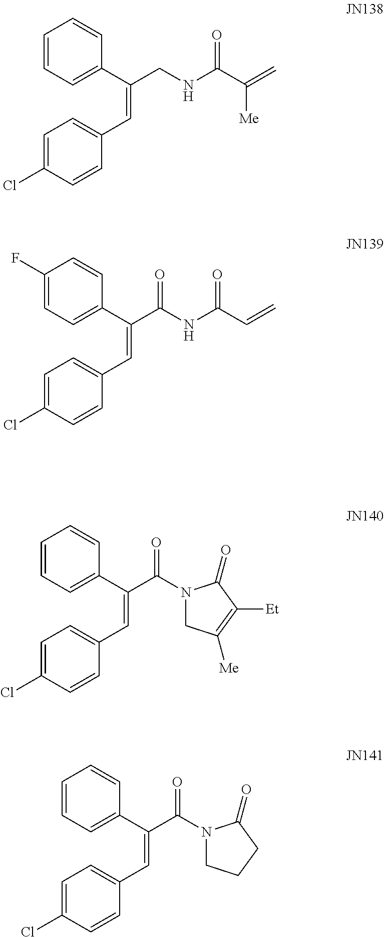 [SMILES: C=C(C)C(=O)NC/C(=C/c1ccc(Cl)cc1)c1ccccc1 ; C=CC(=O)NC(=O)/C(=C/c1ccc(Cl)cc1)c1ccc(F)cc1 ; CCC1=C(C)CN(C(=O)/C(=C/c2ccc(Cl)cc2)c2ccccc2)C1=O ; O=C1CCCN1C(=O)/C(=C/c1ccc(Cl)cc1)c1ccccc1]

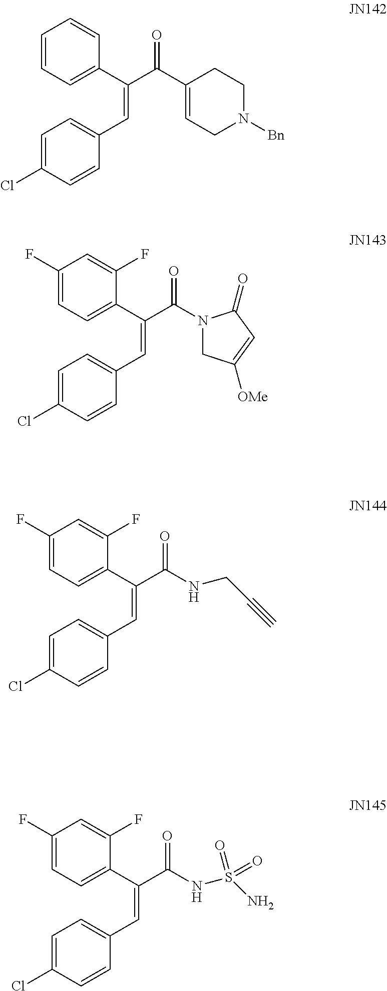 [SMILES: O=C(C1=CCN(Cc2ccccc2)CC1)/C(=C/c1ccc(Cl)cc1)c1ccccc1 ; COC1=CC(=O)N(C(=O)/C(=C/c2ccc(Cl)cc2)c2ccc(F)cc2F)C1 ; C#CCNC(=O)/C(=C/c1ccc(Cl)cc1)c1ccc(F)cc1F ; NS(=O)(=O)NC(=O)/C(=C/c1ccc(Cl)cc1)c1ccc(F)cc1F]

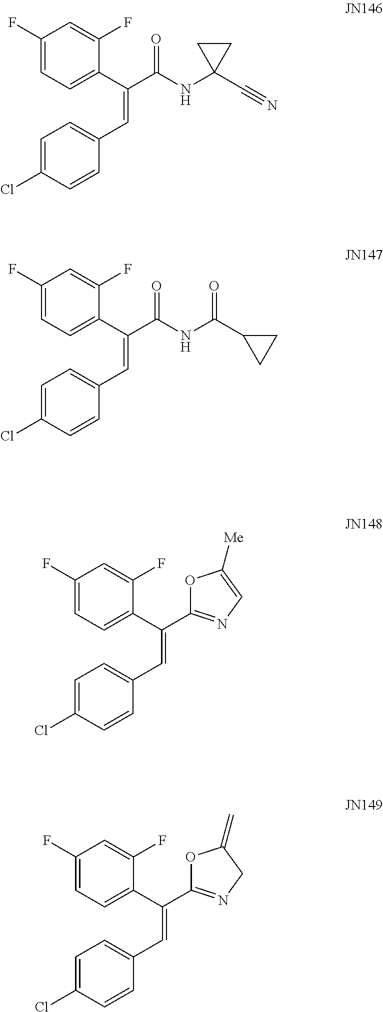 [SMILES: N#CC1(NC(=O)/C(=C/c2ccc(Cl)cc2)c2ccc(F)cc2F)CC1 ; O=C(NC(=O)C1CC1)/C(=C/c1ccc(Cl)cc1)c1ccc(F)cc1F ; Cc1cnc(/C(=C/c2ccc(Cl)cc2)c2ccc(F)cc2F)o1 ; C=C1CN=C(/C(=C/c2ccc(Cl)cc2)c2ccc(F)cc2F)O1]

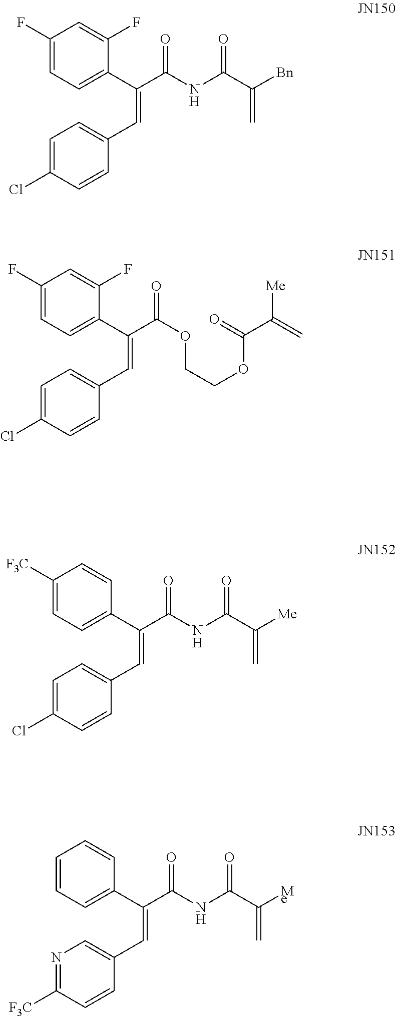 [SMILES: C=C(Cc1ccccc1)C(=O)NC(=O)/C(=C/c1ccc(Cl)cc1)c1ccc(F)cc1F ; C=C(C)C(=O)OCCOC(=O)/C(=C/c1ccc(Cl)cc1)c1ccc(F)cc1F ; C=C(C)C(=O)NC(=O)/C(=C/c1ccc(Cl)cc1)c1ccc(C(F)(F)F)cc1 ; C=C(C)C(=O)NC(=O)/C(=C/c1ccc(C(F)(F)F)nc1)c1ccccc1]

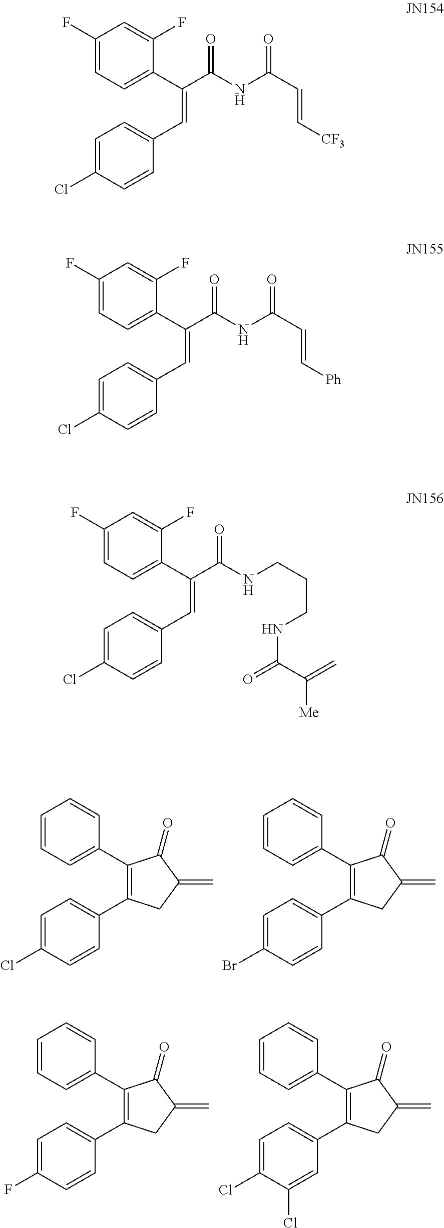

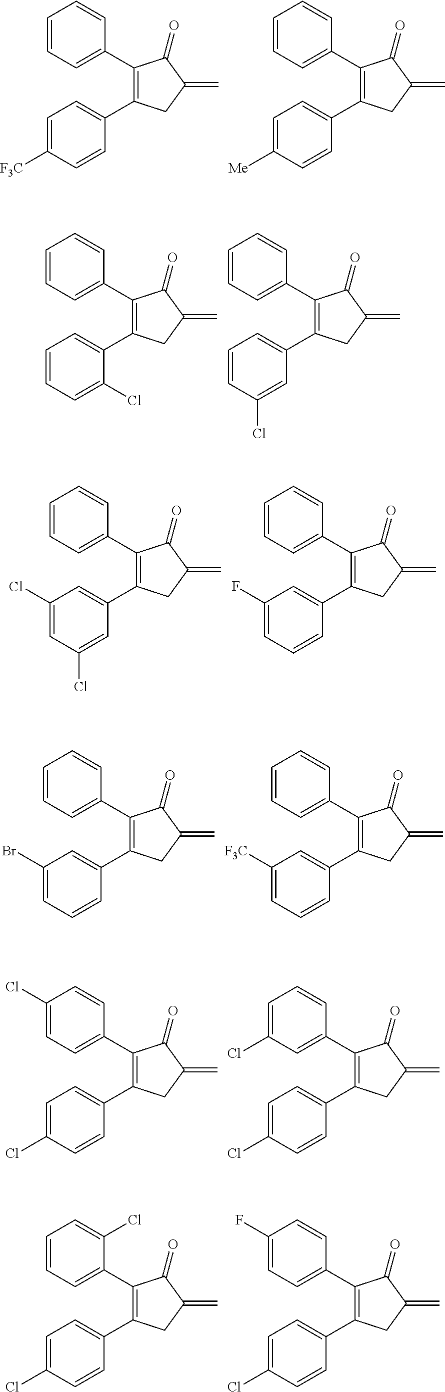

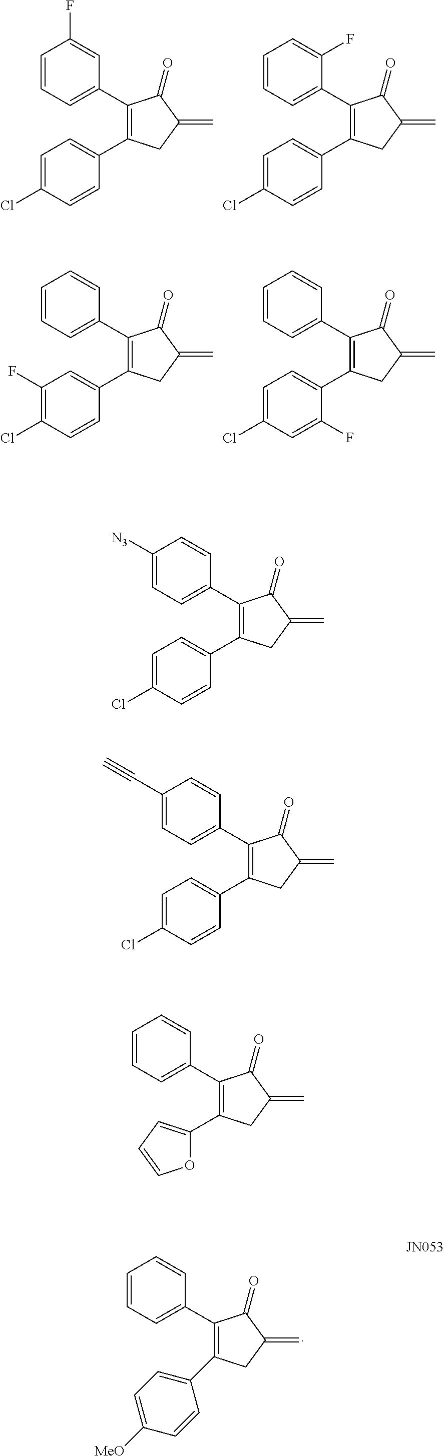

In certain embodiments, the present disclosure provides compounds selected from:

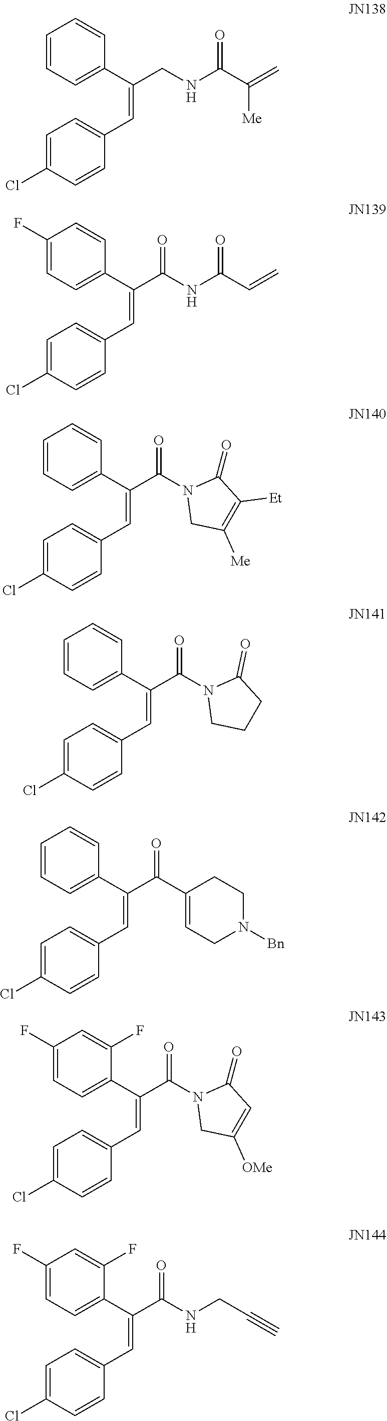

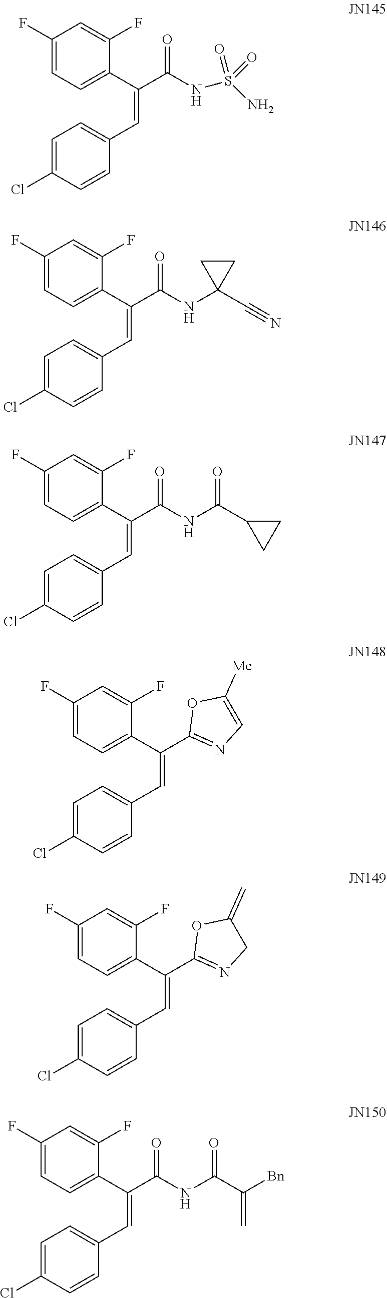 [SMILES: NS(=O)(=O)NC(=O)/C(=C/c1ccc(Cl)cc1)c1ccc(F)cc1F ; N#CC1(NC(=O)/C(=C/c2ccc(Cl)cc2)c2ccc(F)cc2F)CC1 ; O=C(NC(=O)C1CC1)/C(=C/c1ccc(Cl)cc1)c1ccc(F)cc1F ; Cc1cnc(/C(=C/c2ccc(Cl)cc2)c2ccc(F)cc2F)o1 ; C=C1CN=C(/C(=C/c2ccc(Cl)cc2)c2ccc(F)cc2F)O1 ; C=C(Cc1ccccc1)C(=O)NC(=O)/C(=C/c1ccc(Cl)cc1)c1ccc(F)cc1F]

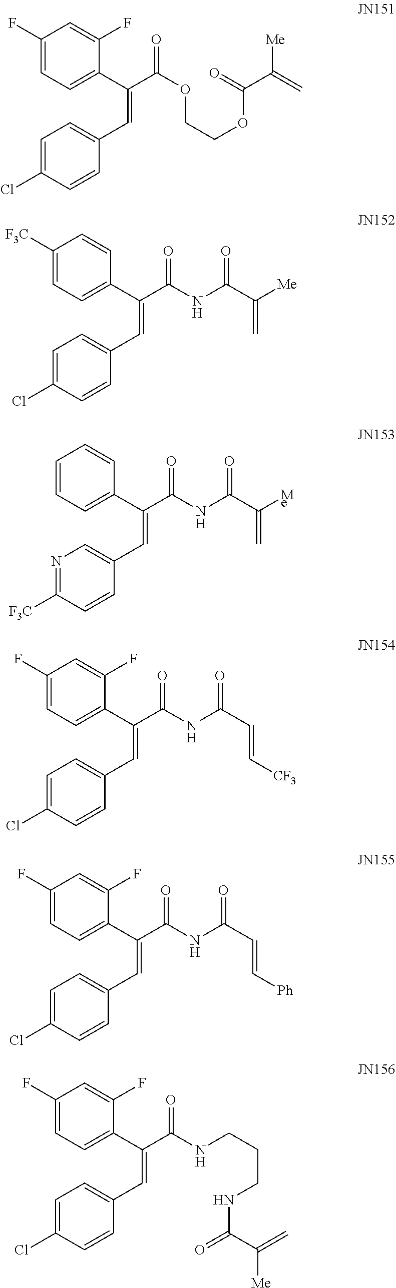

In certain aspects, the present disclosure provides solid forms of compounds disclosed herein.

In certain embodiments, the present disclosure provides Form I of compound JN032

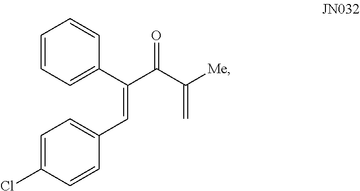 [SMILES: C=C(C)C(=O)/C(=C/c1ccc(Cl)cc1)c1ccccc1]

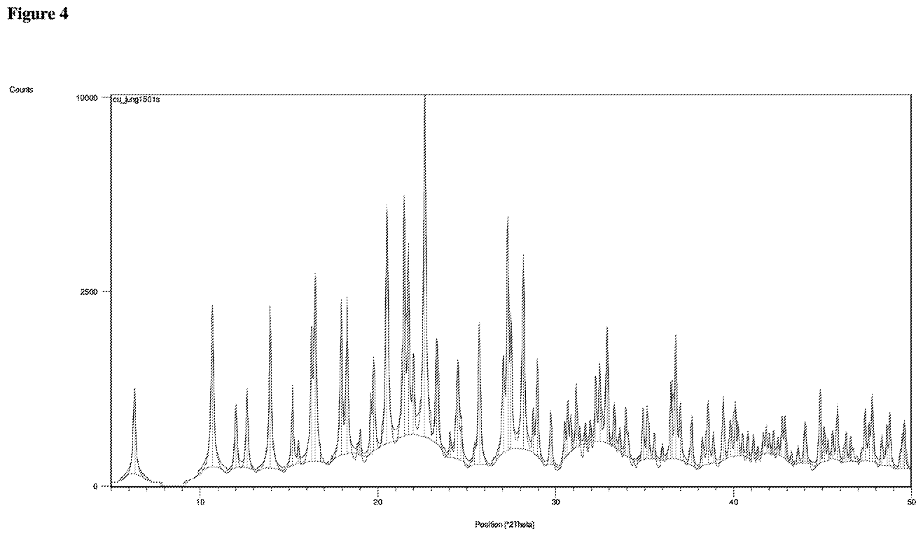

characterized by X-ray powder diffraction peaks at 2θ angles of about 21.5°, about 22.6°, and about 27.3°. In certain embodiments, Form I of JN032 is further characterized by X-ray powder diffraction peaks at 2θ angles of about 16.5°, about 20.5°, and about 28.2°. Form I of JN032 may also be characterized by an X-ray powder diffraction pattern substantially as shown in FIG. 4.

In certain embodiments, the present disclosure provides Form I of compound JN110

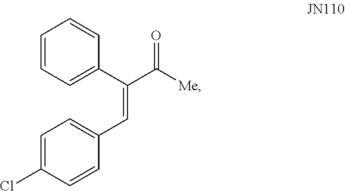 [SMILES: CC(=O)/C(=C/c1ccc(Cl)cc1)c1ccccc1]

characterized by X-ray powder diffraction peaks at 2θ angles of about 17.6°, about 22.2°, and about 28.8°. In certain embodiments, Form I of JN110 is further characterized by X-ray powder diffraction peaks at 2θ angles of about 10.2°, about 15.0°, and about 21.3°. Form I of JN110 may also be characterized by an X-ray powder diffraction pattern substantially as shown in FIG. 5.

In certain embodiments, the present disclosure provides Form I of compound JN034

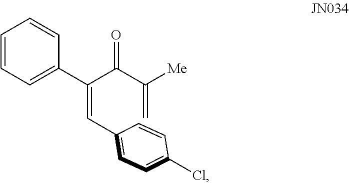

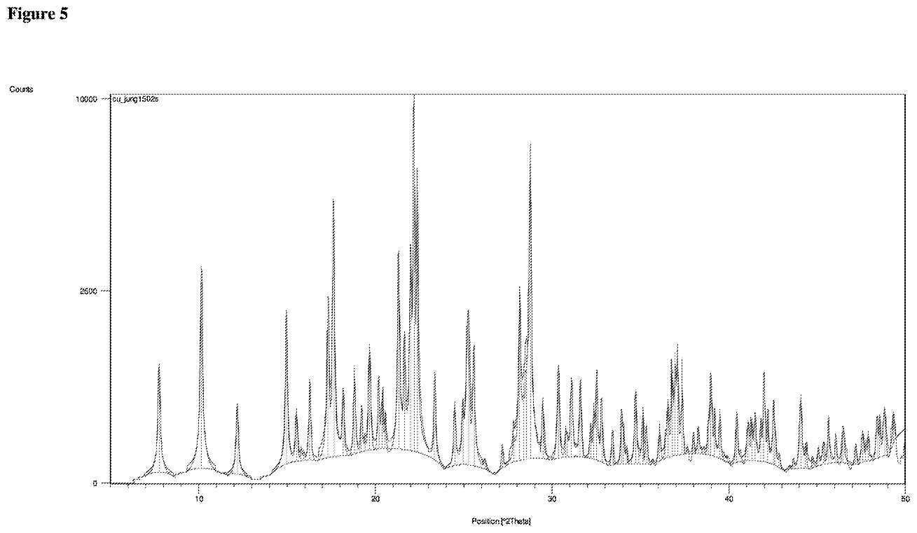

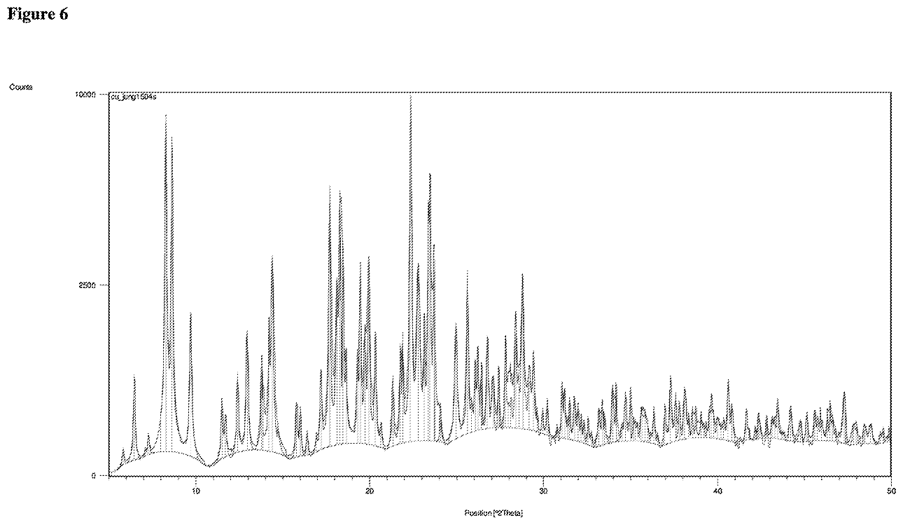

characterized by X-ray powder diffraction peaks at 2θ angles of about 8.3°, about 17.7°, and about 22.4°. In certain embodiments, Form I of JN034 is further characterized by X-ray powder diffraction peaks at 2θ angles of about 9.7°, about 14.4°, and about 25.0°. Form I of JN034 may also be characterized by an characterized by an X-ray powder diffraction pattern substantially as shown in FIG. 6.

In certain embodiments, the present disclosure provides Form I of compound JN097

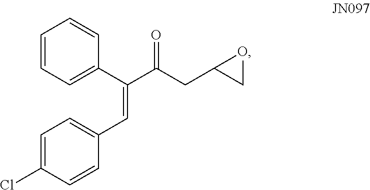 [SMILES: O=C(CC1CO1)/C(=C/c1ccc(Cl)cc1)c1ccccc1]

characterized by X-ray powder diffraction peaks at 2θ angles of about 20.5°, about 23.1°, and about 27.0°. In certain embodiments, Form I of JN097 is further characterized by X-ray powder diffraction peaks at 2θ angles of about 12.1°, about 18.7°, and about 22.1°. Form I of JN097 may also be characterized by an X-ray powder diffraction pattern substantially as shown in FIG. 7.

In certain embodiments, the present disclosure provides Form I of compound JN117

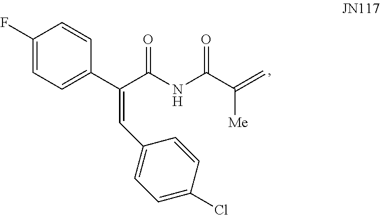 [SMILES: C=C(C)C(=O)NC(=O)/C(=C\c1ccc(Cl)cc1)c1ccc(F)cc1]

characterized by X-ray powder diffraction peaks at 2θ angles of about 7.8°, about 16.4°, and about 21.5°. In certain embodiments, Form I of JN117 is further characterized by X-ray powder diffraction peaks at 2θ angles of about 18.5°, about 19.1°, and about 20.1°. Form I of JN117 may also be characterized by an X-ray powder diffraction pattern substantially as shown in FIG. 8.

In certain embodiments, the present disclosure provides Form I of compound JN103

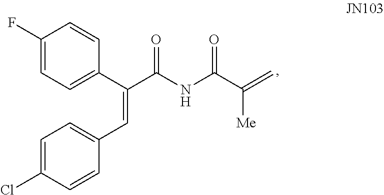 [SMILES: C=C(C)C(=O)NC(=O)/C(=C/c1ccc(Cl)cc1)c1ccc(F)cc1]

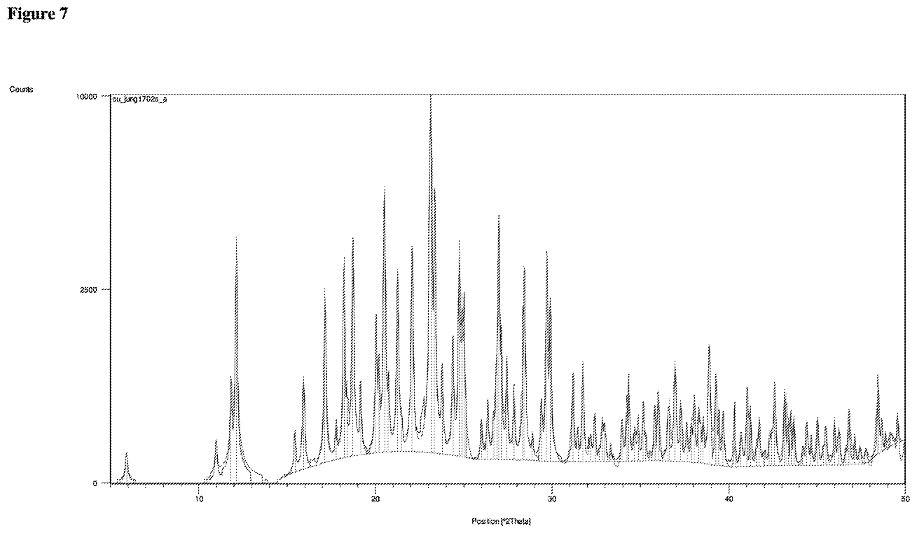

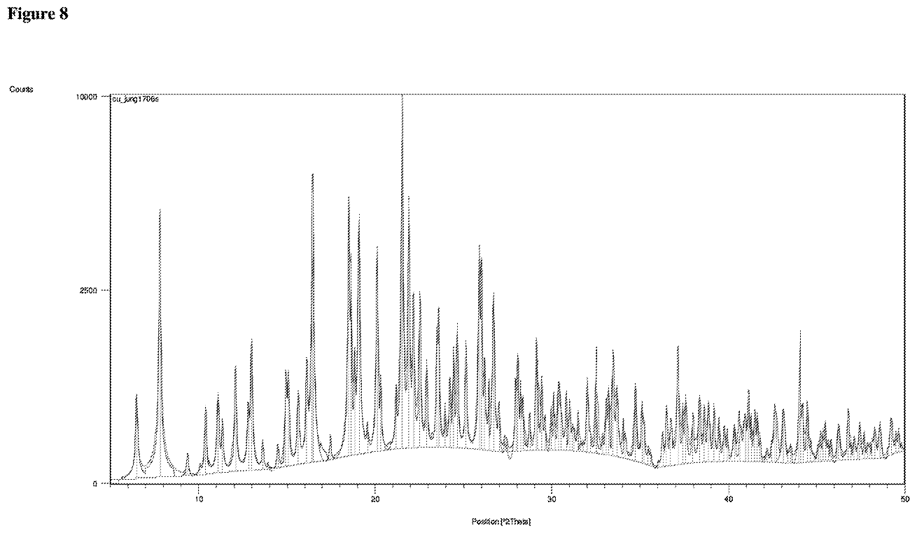

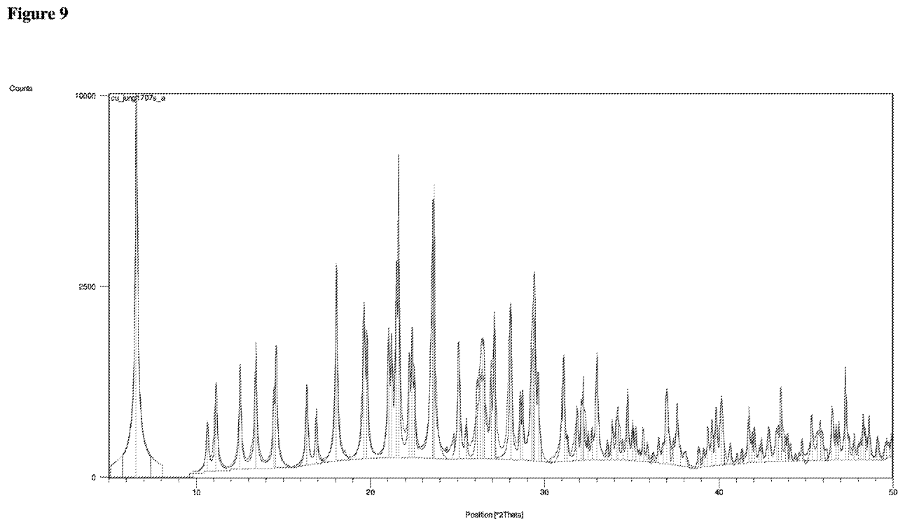

characterized by X-ray powder diffraction peaks at 2θ angles of about 6.6°, about 18.0°, and about 21.6°. In certain embodiments, Form I of JN103 is further characterized by X-ray powder diffraction peaks at 2θ angles of about 23.7°, about 25.1°, and about 28.1°. Form I of JN103 may also be characterized by an X-ray powder diffraction pattern substantially as shown in FIG. 9.

In certain aspects, the present disclosure provides pharmaceutical compositions comprising one of the compounds disclosed herein (such as the solid forms disclosed herein) and a pharmaceutically acceptable excipient.

In certain aspects, the present disclosure provides methods for using of the compounds disclosed herein, for example the solid forms disclosed herein. In certain embodiments, the methods are for inhibiting androgen receptors, and comprise contacting the androgen receptor with a compound or composition disclosed herein. In certain embodiments, the methods are for inducing degradation of an androgen receptor in a cell, comprising contacting the androgen receptor with a compound or composition disclosed herein.

In certain embodiments, the present disclosure provides methods for treating mammals suffering from cancer, comprising administering a compound or composition disclosed herein. In certain embodiments, the cancer is prostate cancer, for example castration-resistant prostate cancer. The cancer may be metastatic or non-metastatic. In certain preferred embodiments, the cancer is resistant to antiandrogen therapy, such as treatment with enzalutamide, bicalutamide, abiraterone, flutamide, nilutamide, darolutamide, or apalutamide. In further embodiments, the cancer is resistant to treatment with enzalutamide, bicalutamide, abiraterone (e.g. abiraterone acetate), flutamide, or nilutamide. In certain such embodiments, the cancer may be resistant to conjoint treatment with abiraterone acetate and prednisone or abiraterone acetate and prednisolone.

In certain aspects, the present disclosure provides compounds as described herein. The compounds described herein are useful, for example, as cancer therapeutics, in particular as AR inhibitors and degraders. In certain aspects, the present disclosure provides methods of treating proliferative diseases, such as prostate cancer, methods of inhibiting AR, and methods of enhancing AR degradation rates using the compounds described herein.

In certain embodiments, compounds of the invention are prodrugs of the compounds described herein. For example, wherein a hydroxyl in the parent compound is presented as an ester or a carbonate, or a carboxylic acid present in the parent compound is presented as an ester. In certain such embodiments, the prodrug is metabolized to the active parent compound in vivo (e.g., the ester is hydrolyzed to the corresponding hydroxyl or carboxylic acid).

In certain embodiments, compounds of the invention may be racemic. In certain embodiments, compounds of the invention may be enriched in one enantiomer. For example, a compound of the invention may have greater than 30% ee, 40% ee, 50% ee, 60% ee, 70% ee, 80% ee, 90% ee, or even 95% or greater ee. In certain embodiments, compounds of the invention may have more than one stereocenter. In certain such embodiments, compounds of the invention may be enriched in one or more diastereomers. For example, a compound of the invention may have greater than 30% de, 40% de, 50% de, 60% de, 70% de, 80% de, 90% de, or even 95% or greater de.

In certain embodiments, the present invention provides pharmaceutical compositions comprising a compound of Formula I, II, III, IV, V, VI, VII, or VIII. In certain embodiments, the pharmaceutical compositions further comprise a pharmaceutically acceptable excipient.

In certain embodiments, the pharmaceutical compositions may be for use in treating or preventing a condition or disease as described herein.

In certain embodiments, the present invention relates to methods of treatment with a compound of Formula I. In certain embodiments, the therapeutic preparation may be enriched to provide predominantly one enantiomer or isomer of a compound. An enantiomerically enriched mixture may comprise, for example, at least 60 mol percent of one enantiomer, or more preferably at least 75, 90, 95, or even 99 mol percent. In certain embodiments, the compound enriched in one enantiomer is substantially free of the other enantiomer, wherein substantially free means that the substance in question makes up less than 10%, or less than 5%, or less than 4%, or less than 3%, or less than 2%, or less than 1% as compared to the amount of the other enantiomer, e.g., in the composition or compound mixture. For example, if a composition or compound mixture contains 98 grams of a first enantiomer and 2 grams of a second enantiomer, it would be said to contain 98 mol percent of the first enantiomer and only 2% of the second enantiomer.

In certain embodiments, the therapeutic preparation may be enriched to provide predominantly one diastereomer of a compound. A diastereomerically enriched mixture may comprise, for example, at least 60 mol percent of one diastereomer, or more preferably at least 75, 90, 95, or even 99 mol percent.

In certain embodiments, the present invention provides a pharmaceutical preparation suitable for use in a human patient, comprising any of the compounds shown above, and one or more pharmaceutically acceptable excipients.

Compounds of any of the above structures may be used in the manufacture of medicaments for the treatment of any diseases or conditions disclosed herein.

In certain aspects, the compounds of the present disclosure are for use in inhibiting an androgen receptor.

In certain aspects, the compounds of the present disclosure are for use in inducing degradation of an androgen receptor in a cell expressing an androgen receptor.

In certain aspects, the compounds of the present disclosure are for use in treating a mammal suffering from cancer. In certain embodiments, the cancer is prostate cancer. In certain embodiments, the cancer is castration-resistant prostate cancer. In certain embodiments, the cancer is metastatic. In certain embodiments, the cancer is non-metastatic.

In certain embodiments of the above aspects, the cancer is resistant to antiandrogen therapy. In certain embodiments, the cancer is resistant to treatment with enzalutamide, bicalutamide, abiraterone, flutamide, or nilutamide. In certain embodiments, the cancer is resistant to treatment with abiraterone acetate. In certain embodiments, the cancer is resistant to conjoint treatment with abiraterone acetate and prednisone.

In certain aspects, the present disclosure provides methods of inhibiting an androgen receptor, comprising contacting the androgen receptor with a compound or composition of the disclosure.

In certain aspects, the present disclosure provides methods of inducing the degradation of an androgen receptor, comprising contacting the androgen receptor with a compound or composition of the disclosure.

In certain aspects, the present disclosure provides methods of treating a mammal suffering from cancer, comprising administering a compound or composition of the disclosure. In certain embodiments, the cancer is prostate cancer. In certain embodiments, the cancer is castration-resistant prostate cancer. In certain embodiments, the cancer is metastatic. In certain embodiments, the cancer is non-metastatic.

In certain embodiments of the above aspects, the cancer is resistant to antiandrogen therapy. In certain embodiments, the cancer is resistant to treatment with enzalutamide, bicalutamide, abiraterone, flutamide, or nilutamide. In certain embodiments, the cancer is resistant to treatment with abiraterone acetate. In certain embodiments, the cancer is resistant to conjoint treatment with abiraterone acetate and prednisone.

### Discussion

The present disclosure describes compounds that inhibit the AR in novel ways. In mammalian cell systems, the compounds of Formulas I, II, III, IV, V, VI, VII, or VIII inhibit ligand-induced and constitutive AR transcriptional activity, and enhance AR degradation.

The compounds disclosed herein target the AR N-terminal TAD. These compounds can be used to treat diseases, the growth of which is driven by the AR or its splice variants. Prostate cancer is an example of one such disease. These compounds offer competitive advantages over existing, approved compounds that target the AR because existing compounds target the LBD of the AR, whereas the compounds disclosed herein are active against full length and constitutively active AR variants that lack a functional LBD. The compounds disclosed herein target the AR N-terminus and inhibits the activity of constitutively active AR variants that lack a functional LBD (see below, section 6 for more details). These AR variants have been shown to confer resistance to currently approved AR targeting agents. In addition, these compounds induce degradation of the AR including AR splice variants, which is not a known mechanism of any AR targeting agent that has received regulatory approval. These AR variants have been shown to confer resistance to current AR targeting agents.

### Compositions and Modes of Administration

The compounds of this invention may be used in treating the conditions described herein, in the form of the free base, salts (preferably pharmaceutically acceptable salts), solvates, hydrates, prodrugs, isomers, or mixtures thereof. All forms are within the scope of the disclosure. Acid addition salts may be formed and provide a more convenient form for use; in practice, use of the salt form inherently amounts to use of the base form. The acids which can be used to prepare the acid addition salts include preferably those which produce, when combined with the free base, pharmaceutically acceptable salts, that is, salts whose anions are non-toxic to the subject organism in pharmaceutical doses of the salts, so that the beneficial properties inherent in the free base are not vitiated by side effects ascribable to the anions. Although pharmaceutically acceptable salts of the basic compounds are preferred, all acid addition salts are useful as sources of the free base form even if the particular salt per se is desired only as an intermediate product as, for example, when the salt is formed only for the purposes of purification and identification, or when it is used as an intermediate in preparing a pharmaceutically acceptable salt by ion exchange procedures.

Pharmaceutically acceptable salts within the scope of the disclosure include those derived from the following acids; mineral acids such as hydrochloric acid, sulfuric acid, phosphoric acid and sulfamic acid; and organic acids such as acetic acid, citric acid, lactic acid, tartaric acid, malonic acid, methanesulfonic acid, ethanesulfonic acid, benzenesulfonic acid, p-toluenesulfonic acid, cyclohexylsulfamic acid, quinic acid, and the like.

The compounds of the present invention can be formulated as pharmaceutical compositions and administered to a subject in need of treatment, for example a mammal, such as a human patient, in a variety of forms adapted to the chosen route of administration, for example, orally, nasally, intraperitoneally, or parenterally (e.g., by intravenous, intraperitoneal, subcutaneous, intramuscular, transepithelial, nasal, intrapulmonary, intrathecal, rectal or topical routes). Parenteral administration may be by continuous infusion over a selected period of time.

In accordance with the methods of the disclosure, the described compounds may be administered to a patient in a variety of forms depending on the selected route of administration, as will be understood by those skilled in the art. The compositions containing the compounds of the disclosure can be prepared by known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the substances in association with one or more pharmaceutically acceptable vehicles or diluents, and contained in buffered solutions with a suitable pH and iso-osmotic with the physiological fluids.

A composition comprising a compound of the present disclosure may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

A person skilled in the art would know how to prepare suitable formulations. Conventional procedures and ingredients for the selection and preparation of suitable formulations are described, for example, in Remington's Pharmaceutical Sciences (1990-18th edition) and in The United States Pharmacopeia: The National Formulary (USP 24 NF19) published in 1999.

Thus, compounds of the invention may be systemically administered, e.g., orally, in combination with a pharmaceutically acceptable vehicle such as an inert diluent or an assimilable edible carrier; or by inhalation or insufflation. They may be enclosed in hard or soft shell gelatin capsules, may be compressed into tablets, or may be incorporated directly with the food of the patient's diet. For oral therapeutic administration, the compounds may be combined with one or more excipients and used in the form of ingestible tablets, buccal tablets, troches, capsules, elixirs, suspensions, syrups, wafers, and the like. The compounds may be combined with a fine inert powdered carrier and inhaled by the subject or insufflated. Such compositions and preparations should contain at least 0.1% of compounds of formulas I, II, III, IV, V, VI, VII, or VIII. The percentage of the compositions and preparations may, of course, be varied and may conveniently be between about 2% to about 60% of the weight of a given unit dosage form. The amount of the compounds in such therapeutically useful compositions is such that an effective dosage level will be obtained.

In certain embodiments of the disclosure, compositions comprising a compound of the present disclosure for oral administration include capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and the like, each containing a predetermined amount of the compound of the present disclosure as an active ingredient.

In solid dosage forms for oral administration (capsules, tablets, troches, pills, dragees, powders, granules, and the like), one or more compositions comprising the compound of the present disclosure may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, gum tragacanth, corn starch, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Any material used in preparing any unit dosage form should be pharmaceutically acceptable and substantially non-toxic in the amounts employed. In addition, the compounds may be incorporated into sustained-release preparations and devices. For example, the compounds may be incorporated into time release capsules, time release tablets, and time release pills.

Liquid dosage forms for oral administration include pharmaceutically acceptable emulsions, microemulsions, solutions, suspensions, syrups, and elixirs. In addition to the compound of the present disclosure, the liquid dosage forms may contain inert diluents commonly used in the art, such as water or other solvents, solubilizing agents and emulsifiers, such as ethyl alcohol (ethanol), isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

Suspensions, in addition to the active compounds, salts and/or prodrugs thereof, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

In certain embodiments, pharmaceutical compositions suitable for parenteral administration may comprise the compound of the present disclosure in combination with one or more pharmaceutically acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and non-aqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

The compounds may be administered intravenously or intraperitoneally by infusion or injection. Solutions of the compounds or their salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the compounds which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the compounds in the required amount in the appropriate solvent with various of the other ingredients enumerated above, as required, followed by filter sterilization. In the case of sterile powders for the preparation of sterile injectable solutions, the preferred methods of preparation are vacuum drying and freeze drying techniques, which yield a powder of the active ingredient plus any additional desired ingredient present in the previously sterile-filtered solutions.

For topical administration, the compounds may be applied in pure form. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Other solid carriers include nontoxic polymeric nanoparticles or microparticles. Useful liquid carriers include water, alcohols or glycols or water/alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508), all of which are hereby incorporated by reference.

Useful dosages of the compounds of formulas I, II, III, IV, V, VI, VII, or VIII can be determined by comparing their in vitro activity, and in vivo activity in animal models. Methods for the extrapolation of effective dosages in mice, and other animals, to humans are known to the art; for example, see U.S. Pat. No. 4,938,949, which is hereby incorporated by reference.

For example, the concentration of the compounds in a liquid composition, such as a lotion, can be from about 0.1-25% by weight, or from about 0.5-10% by weight. The concentration in a semi-solid or solid composition such as a gel or a powder can be about 0.1-5% by weight, or about 0.5-2.5% by weight.

The amount of the compounds required for use in treatment will vary not only with the particular salt selected but also with the route of administration, the nature of the condition being treated and the age and condition of the patient and will be ultimately at the discretion of the attendant physician or clinician.

Effective dosages and routes of administration of agents of the invention are conventional. The exact amount (effective dose) of the agent will vary from subject to subject, depending on, for example, the species, age, weight and general or clinical condition of the subject, the severity or mechanism of any disorder being treated, the particular agent or vehicle used, the method and scheduling of administration, and the like. A therapeutically effective dose can be determined empirically, by conventional procedures known to those of skill in the art. See, e.g., The Pharmacological Basis of Therapeutics, Goodman and Gilman, eds., Macmillan Publishing Co., New York. For example, an effective dose can be estimated initially either in cell culture assays or in suitable animal models. The animal model may also be used to determine the appropriate concentration ranges and routes of administration. Such information can then be used to determine useful doses and routes for administration in humans. A therapeutic dose can also be selected by analogy to dosages for comparable therapeutic agents.

The particular mode of administration and the dosage regimen will be selected by the attending clinician, taking into account the particulars of the case (e.g., the subject, the disease, the disease state involved, and whether the treatment is prophylactic). Treatment may involve daily or multi-daily doses of compound(s) over a period of a few days to months, or even years.

In general, however, a suitable dose will be in the range of from about 0.001 to about 100 mg/kg, e.g., from about 0.01 to about 100 mg/kg of body weight per day, such as above about 0.1 mg per kilogram, or in a range of from about 1 to about 10 mg per kilogram body weight of the recipient per day. For example, a suitable dose may be about 1 mg/kg, 10 mg/kg, or 50 mg/kg of body weight per day.

The compounds of formulas I, II, III, IV, V, VI, VII, or VIII are conveniently administered in unit dosage form; for example, containing 0.05 to 10000 mg, 0.5 to 10000 mg, 5 to 1000 mg, or about 100 mg of active ingredient per unit dosage form.

The compounds can be administered to achieve peak plasma concentrations of, for example, from about 0.5 to about 75 μM, about 1 to 50 μM, about 2 to about 30 μM, or about 5 to about 25 μM. Exemplary desirable plasma concentrations include at least or no more than 0.25, 0.5, 1, 5, 10, 25, 50, 75, 100 or 200 μM. For example, plasma levels may be from about 1 to 100 micromolar or from about 10 to about 25 micromolar. This may be achieved, for example, by the intravenous injection of a 0.05 to 5% solution of the compounds, optionally in saline, or orally administered as a bolus containing about 1-100 mg of the compounds. Desirable blood levels may be maintained by continuous infusion to provide about 0.00005-5 mg per kg body weight per hour, for example at least or no more than 0.00005, 0.0005, 0.005, 0.05, 0.5, or 5 mg/kg/hr. Alternatively, such levels can be obtained by intermittent infusions containing about 0.0002-20 mg per kg body weight, for example, at least or no more than 0.0002, 0.002, 0.02, 0.2, 2, 20, or 50 mg of the compounds per kg of body weight.

The compounds may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator.

The dosage of the compounds and/or compositions of the disclosure can vary depending on many factors such as the pharmacodynamic properties of the compound, the mode of administration, the age, health and weight of the recipient, the nature and extent of the symptoms, the frequency of the treatment and the type of concurrent treatment, if any, and the clearance rate of the compound in the subject to be treated. One of skill in the art can determine the appropriate dosage based on the above factors. The compounds of the disclosure may be administered initially in a suitable dosage that may be adjusted as required, depending on the clinical response. To calculate the human equivalent dose (HED) from a dosage used in the treatment of age-dependent cognitive impairment in rats, the formula HED (mg/kg)=rat dose (mg/kg)×0.16 may be employed (see Estimating the Safe Starting Dose in Clinical Trials for Therapeutics in Adult Healthy Volunteers, December 2002, Center for Biologics Evaluation and Research). For example, using that formula, a dosage of 10 mg/kg in rats is equivalent to 1.6 mg/kg in humans. This conversion is based on a more general formula HED=animal dose in mg/kg×(animal weight in kg/human weight in kg) 0.33. Similarly, to calculate the HED from a dosage used in the treatment in mouse, the formula HED (mg/kg)=mouse dose (mg/kg)×0.08 may be employed (see Estimating the Safe Starting Dose in Clinical Trials for Therapeutics in Adult Healthy Volunteers, December 2002, Center for Biologics Evaluation and Research).

The compounds and/or compositions of the disclosure can be used alone or conjointly with other therapeutic agents, or in combination with other types of treatment for treating cell proliferative disorders such as prostate cancer. For example, in some embodiments, the compounds and compositions of the disclosure can be used for treating CRPC or for treating cancers that are resistant to antiandrogen therapies such as enzalutamide, bicalutamide, abiraterone, flutamide, or nilutamide. For example, these other therapeutically useful agents may be administered in a single formulation, simultaneously or sequentially with the compound of the present disclosure according to the methods of the disclosure.

A number of the above-identified compounds exhibit little or no agonistic activities with respect to hormone refractory prostate cancer cells. Because these compounds are strong AR inhibitors, they can be used not only in treating prostate cancer, but also in treating other AR related diseases or conditions such as benign prostate hyperplasia, hair loss, and acne. Because AR belongs to the family of nuclear receptors, these compounds may serve as scaffolds for drug synthesis targeting other nuclear receptors, such as estrogen receptor and peroxisome proliferator-activated receptor. Therefore, they may be further developed for other diseases such as breast cancer, ovarian cancer, diabetes, cardiac diseases, and metabolism related diseases, in which nuclear receptors play a role.

### Crystalline Forms

In certain aspects, the present invention provides solid forms of the compounds described herein. In certain preferred embodiments, the solid form is a crystalline form. A crystalline form of a compound described herein can be used to facilitate purification of the compound (e.g., through recrystallization) and/or to modulate/improve the physicochemical properties of the compound, including but not limited to solid state properties (e.g., crystallinity, hygroscopicity, melting point, or hydration), pharmaceutical properties (e.g., solubility/dissolution rate, stability, or compatibility), as well as crystallization characteristics (e.g., purity, yield, or morphology).

In certain aspects, the present invention provides a solid form of compound JN032

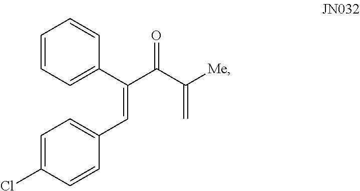 [SMILES: C=C(C)C(=O)/C(=C/c1ccc(Cl)cc1)c1ccccc1]

characterized by X-ray powder diffraction (XRPD) peaks at 2θ angles of about 21.5°, about 22.6°, and about 27.3°. In certain preferred embodiments, the solid form of compound JN032 is characterized by an XRPD diffraction pattern substantially as shown in FIG. 4.

In certain aspects, the present invention provides a solid form, which is Form I of compound JN11C

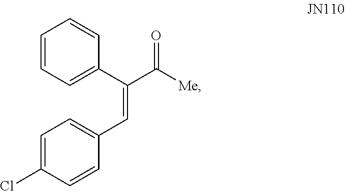 [SMILES: CC(=O)/C(=C/c1ccc(Cl)cc1)c1ccccc1]

characterized by XRPD peaks at 2θ angles of about 17.6°, about 22.2°, and about 28.8°. In certain preferred embodiments, the solid form of compound JN110 is characterized by an XRPD pattern substantially as shown in FIG. 5.

In certain aspects, the present invention provides a solid form, which is Form I of compound JN034

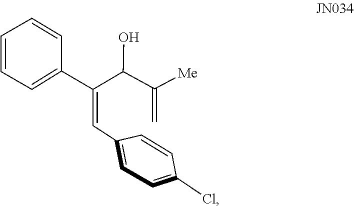

characterized by XRPD peaks at 2θ angles of about 8.3°, about 17.7°, and about 22.4°. In certain preferred embodiments, the solid form of compound JN034 is characterized by an XRPD pattern substantially as shown in FIG. 6.

In certain aspects, the present invention provides a solid form, which is Form I of compound JN097

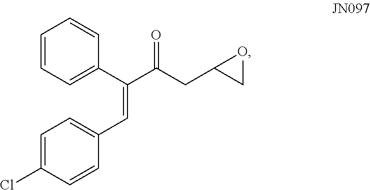 [SMILES: O=C(CC1CO1)/C(=C/c1ccc(Cl)cc1)c1ccccc1]

characterized by XRPD peaks at 2θ angles of about 20.5°, about 23.1°, and about 27.0°. In certain preferred embodiments, the solid form of compound JN097 is characterized by an XRPD pattern substantially as shown in FIG. 7.

In certain aspects, the present invention provides a solid form, which is Form I of compound JN117

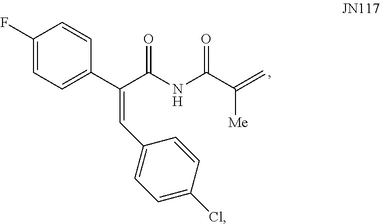 [SMILES: C=C(C)C(=O)NC(=O)/C(=C\c1ccc(Cl)cc1)c1ccc(F)cc1]

characterized by XRPD peaks at 2θ angles of about 7.8°, about 16.4°, and about 21.5°. In certain preferred embodiments, the solid form of compound JN117 is characterized by an XRPD pattern substantially as shown in FIG. 8.

In certain aspects, the present invention provides a solid form, which is Form I of compound JN103

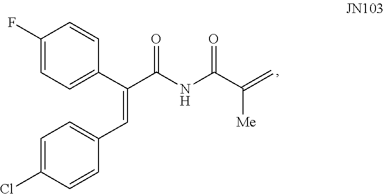 [SMILES: C=C(C)C(=O)NC(=O)/C(=C/c1ccc(Cl)cc1)c1ccc(F)cc1]

characterized by XRPD peaks at 2θ angles of about 6.6°, about 18.0°, and about 21.6°. In certain preferred embodiments, the solid form of compound JN103 is characterized by an XRPD pattern substantially as shown in FIG. 9.

The relative intensity, as well as the two theta value, of each peak FIGS. 4-9 may change or shift under certain conditions, although the crystalline form is the same. One of ordinary skill in the art should be able to readily determine whether a given crystalline form is the same crystalline form as described in one of FIGS. 4-9 by comparing their XRPD data. As used herein, a XRPD dataset is “substantially as shown in” another XRPD dataset if one or more of the peaks in one dataset are within ±0.2° 2θ of the corresponding peaks in the other dataset.

As used herein, the term “about” is defined as being close to as understood by one of ordinary skill in the art. In one non-limiting embodiment, when used in reference to amounts or volumes of compounds, reagents, or solvents, the term “about” is defined to be within 10%, preferably within 5%, more preferably within 1%, and most preferably within 0.5%. In another non-limiting embodiment, when used in reference to XRPD peaks, a peak is at “about” a recited value if the peak is within ±0.2° 2θ of the recited value.

In certain embodiments, the crystalline form is substantially pure. As used herein, the term “substantially pure”, when used in reference to a given crystalline form, refers to the crystalline form which is at least about 90% pure. This means that the crystalline form does not contain more than about 10% of any other form of the compound. More preferably, the term “substantially pure” refers to a crystalline form of the compound which is at least about 95% pure. This means that the crystalline form of the compound does not contain more than about 5% of any other form of the compound. Even more preferably, the term “substantially pure” refers to a crystalline form of the compound which is at least about 97% pure. This means that the crystalline form of the compound does not contain more than about 3% of any other form of the compound.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, Mass. (2000).

Chemistry terms used herein are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known. The ability of such agents to inhibit AR or promote AR degradation may render them suitable as “therapeutic agents” in the methods and compositions of this disclosure.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, occularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g. solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

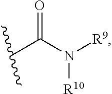

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

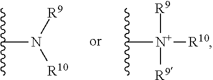

wherein R⁹, R¹⁰, and R¹⁰′ each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

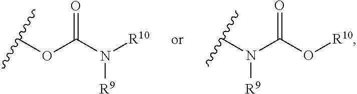

wherein R⁹ and R¹⁰ independently represent hydrogen or a hydrocarbyl group.

The term “carbocyclylalkyl”, as used herein, refers to an alkyl group substituted with a carbocycle group.

The terms “carbocycle”, “carbocyclyl”, and “carbocyclic”, as used herein, refers to a non-aromatic saturated or unsaturated ring in which each atom of the ring is carbon. Preferably a carbocycle ring contains from 3 to 10 atoms, more preferably from 5 to 7 atoms.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

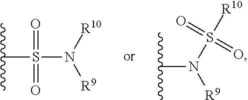

wherein R⁹ and R¹⁰ independently represents hydrogen or hydrocarbyl.

The term “sulfoxide” is art-recognized and refers to the group —S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹ or —SC(O)R⁹

wherein R⁹ represents a hydrocarbyl.

The term “thioether”, as used herein, is equivalent to an ether, wherein the oxygen is replaced with a sulfur.

The term “urea” is art-recognized and may be represented by the general formula

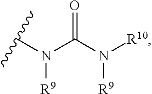

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by formulas I, II, III, IV, V, VI, VII, or VIII. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of formulas I, II, III, IV, V, VI, VII, or VIII are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of formulas I, II, III, IV, V, VI, VII, or VIII for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by formulas I, II, III, IV, V, VI, VII, or VIII or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form the compound of the present disclosure (e.g., compounds of formulas I, II, III, IV, V, VI, VII, or VIII). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of formulas I, II, III, IV, V, VI, VII, or VIII. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “Log of solubility”, “LogS” or “logS” as used herein is used in the art to quantify the aqueous solubility of a compound. The aqueous solubility of a compound significantly affects its absorption and distribution characteristics. A low solubility often goes along with a poor absorption. LogS value is a unit stripped logarithm (base 10) of the solubility measured in mol/liter.

### DISCUSSION

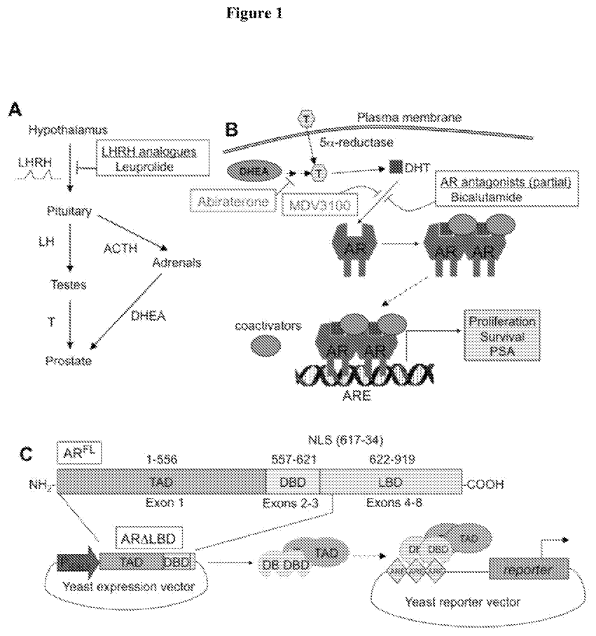

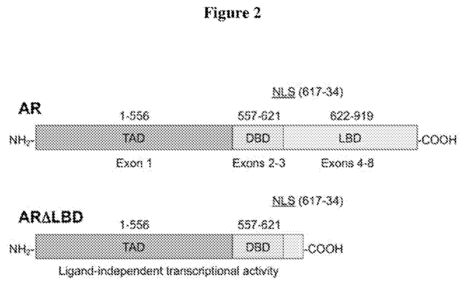

Adenocarcinoma of the prostate (PCa) is the most common non-cutaneous solid tumor diagnosed in men in the U.S. and represents the second leading cause of cancer-related mortality in men, second only to lung cancer. PCa is initially androgen dependent (AD), and androgen deprivation therapy (ADT), which is delivered by surgical or chemical castration in the form of luteinizing hormone releasing hormone (LHRH) analogues (FIG. 1A), results in apoptosis and growth arrest of AD PCa cells and induces a clinical response in virtually all patients. Unfortunately, castration resistant prostate cancer (CRPC) inevitably develops and not only represents the terminal phase of the disease with a median survival of approximately 12-15 months, but also is associated with profound morbidity. Until recently, the chemotherapeutic agent, docetaxel, was the only systemic therapy for CRPC that prolonged median overall survival, albeit by a modest two to three months. In 2010, another cytotoxic chemotherapeutic, cabazitaxel, was also granted regulatory approval for docetaxel-resistant patients based on a three month improvement in survival, as was the cellular vaccine, Provenge, which extended survival by four months in a highly select sub-group of patients with excellent performance status. Thus, despite these modest, incremental advances, novel treatment approaches based on an understanding of the biology behind castration resistance are required to more substantially improve the outcomes of CRPC patients.

A large body of experimental and clinical evidence has established that restoration of AR activity underlies therapeutic resistance in the vast majority of CRPC patients. Although the AR has non-genotropic effects, reactivation of AR transcriptional activity represents the principal biochemical driving force that is necessary and sufficient for castration resistance. Cellular adaptations, including 1) AR gene amplification, 2) intratumoral steroidogenesis, 3) gain-of-function AR gene mutations that allow for ligand promiscuity, 4) somatic mosaicism of the AR, 5) heightened expression of AR transcriptional coactivators, 6) as well as truly ligand-independent AR activation mediated by growth factors, cytokines, and AR phosphorylation, are mutually non-exclusive mechanisms that drive AR transcriptional activity despite castrate serum levels of androgens. Activating mutations of the AR signaling axis has been identified in nearly all cases of CRPC in a recent integrative genomic analysis of over 200 CRPC patients.

Based on these observations, drugs that target the AR signaling axis through novel approaches, including pure AR antagonists (e.g. enzalutamide) and CYP17 inhibitors aimed at inhibiting intratumoral steroidogenesis (e.g. abiraterone acetate) have made their way through the clinic (FIG. 1B). Abiraterone acetate and enzalutamide have both been approved for the treatment of metastatic CRPC (mCRPC). However, primary resistance to these agents occurs in roughly one third of patients, while the remaining patients develop secondary resistance manifested by progression of disease after an initial period of response of variable duration.

The phase 3 studies that demonstrated the clinical success of abiraterone acetate and enzalutamide in chemotherapy naïve and post-chemotherapy patients confirmed the pathophysiologic relevance of the AR as a driver of castration resistance. Cross-resistance between abiraterone and enzalutamide is the norm as evidenced by the low response rate when one of these agents is used subsequent to progression on the other. Since the clinical implementation of these second-generation endocrine therapies, pre-clinical models as well as sequencing studies of cohorts of mCRPC patients have demonstrated ongoing AR expression and signaling in post-abiraterone/post-enzalutamide mCRPC. In fact, the AR is the most frequently mutated gene, and an AR-dependent transcriptional program is reactivated in this context. Thus, the AR represents a key driver of castration resistant growth in both newly developed CRPC and post-abiraterone/post-enzalutamide CRPC.

Constitutively active variants of the AR that lack a functional LBD have recently been shown to be expressed in prostate cancer specimens with increasing frequency in mCRPC specimens. These constitutively active variants confer resistance to abiraterone acetate and enzalutamide; in fact, these variants would not be expected to respond to any existing drug that directly or indirectly targets the LBD. Given the inevitable development of primary or secondary resistance to abiraterone and enzalutamide and the pathophysiologic relevance of the AR throughout the natural and treated history of the castration resistant state, there is an unmet need to develop novel AR targeting agents to improve the clinical outcomes of patients with metastatic CRPC.

All existing endocrine therapies in clinical use for the treatment of PCa, including but not limited to abiraterone and enzalutamide, directly or indirectly target the C-terminal ligand binding domain (LBD) of the AR. The C-terminal LBD of the AR represents the direct or indirect molecular target of new AR targeting agents in development as well as those that have long been employed, including luteinizing hormone releasing hormone (LHRH) analogues (e.g. leuprolide, a “chemical castration”) and partial AR antagonists (e.g. bicalutamide) (FIG. 1C). The other major domains of the AR, including the centrally located DNA binding domain (DBD) and N-terminal transactivation domain (TAD), have yet to be directly targeted and exploited for therapeutic benefit. These domains are required for AR transcriptional activity, yet no drug that targets either of these domains has been successfully brought to the point of regulatory approval to date. The centrally located DBD shares significant homology with other members of the nuclear steroid receptor family (e.g. glucocorticoid receptor [GR], progesterone receptor [PR]), whereas the N-terminally located AR TAD shares the least homology with that of other members of this family and accordingly could be selectively targeted.

The AR TAD is an intrinsically disordered protein that has not been amenable to crystallization. Hence, its structure has not been resolved, and, by extension, the AR TAD does not lend itself to structure based drug design. Proof-of-principle support for the notion of targeting the TAD has come from studies in which TAD decoy molecules inhibited AR-dependent growth.

Proof-of-principle support for the notion of targeting the TAD has come from recent studies by a group that identified TAD decoy molecules as well as a marine sponge extract that selectively targets the AR TAD. Importantly, this marine sponge extract, known as EPI-001, inhibited CRPC growth through interaction with the AF1 region of the TAD. EPI-001 was not identified through a high throughput screen, and is likely to have been absorbed by marine sponges in vivo as an industrial compound. Other compounds have been shown to have an inhibitory effect on constitutively active AR splice variants. Galeterone binds to the AR LBD but was reported to induce degradation of AR splice variants. Galeterone entered into clinical trials, but a phase 3 studied was recently discontinued at an interim analysis due to futility. Niclosamide, an anti-fungal agent, also inhibits AR splice variants and has entered into early phase clinical trials. Other AR TAD inhibitors include those described in International Publication No. WO 2018/136792, which is fully incorporated herein by reference.

The compounds disclosed herein have been prepared and tested for activity against AR, as listed in Table 1:

TABLE 1

| | |
|---|---|
| 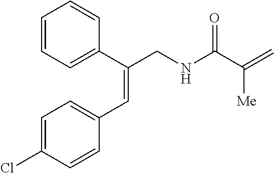 [SMILES: C=C(C)C(=O)NC/C(=C/c1ccc(Cl)cc1)c1ccccc1] | JN138 |
| 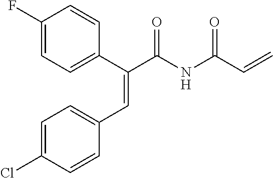 [SMILES: C=CC(=O)NC(=O)/C(=C/c1ccc(Cl)cc1)c1ccc(F)cc1] | JN139 |
| 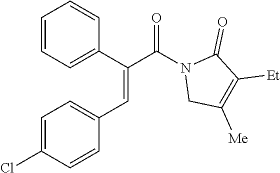 [SMILES: CCC1=C(C)CN(C(=O)/C(=C/c2ccc(Cl)cc2)c2ccccc2)C1=O] | JN140 |
| 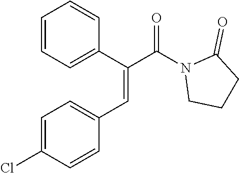 [SMILES: O=C1CCCN1C(=O)/C(=C/c1ccc(Cl)cc1)c1ccccc1] | JN141 |
| 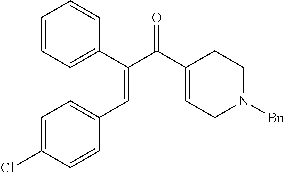 [SMILES: O=C(C1=CCN(Cc2ccccc2)CC1)/C(=C/c1ccc(Cl)cc1)c1ccccc1] | JN142 |
| 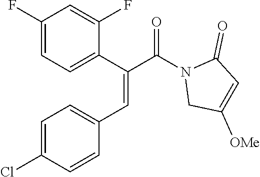 [SMILES: COC1=CC(=O)N(C(=O)/C(=C/c2ccc(Cl)cc2)c2ccc(F)cc2F)C1] | JN143 |
| 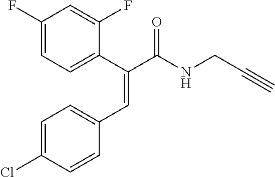 [SMILES: C#CCNC(=O)/C(=C/c1ccc(Cl)cc1)c1ccc(F)cc1F] | JN144 |
| 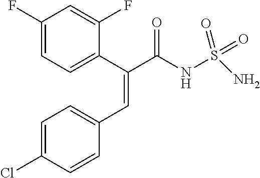 [SMILES: NS(=O)(=O)NC(=O)/C(=C/c1ccc(Cl)cc1)c1ccc(F)cc1F] | JN145 |
| 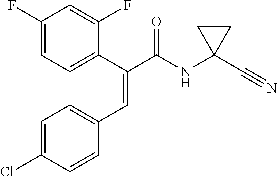 [SMILES: N#CC1(NC(=O)/C(=C/c2ccc(Cl)cc2)c2ccc(F)cc2F)CC1] | JN146 |
| 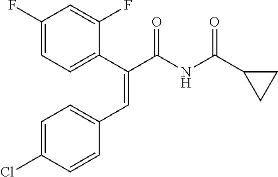 [SMILES: O=C(NC(=O)C1CC1)/C(=C/c1ccc(Cl)cc1)c1ccc(F)cc1F] | JN147 |
| 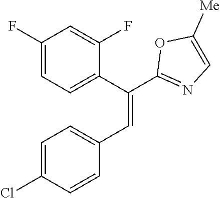 [SMILES: Cc1cnc(/C(=C/c2ccc(Cl)cc2)c2ccc(F)cc2F)o1] | JN148 |
| 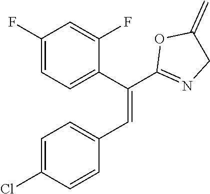 [SMILES: C=C1CN=C(/C(=C/c2ccc(Cl)cc2)c2ccc(F)cc2F)O1] | JN149 |
| 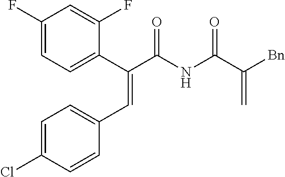 [SMILES: C=C(Cc1ccccc1)C(=O)NC(=O)/C(=C/c1ccc(Cl)cc1)c1ccc(F)cc1F] | JN150 |
| 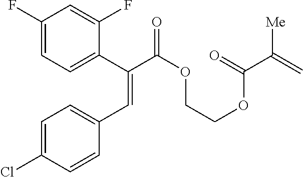 [SMILES: C=C(C)C(=O)OCCOC(=O)/C(=C/c1ccc(Cl)cc1)c1ccc(F)cc1F] | JN151 |
| 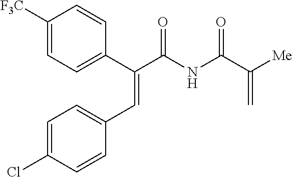 [SMILES: C=C(C)C(=O)NC(=O)/C(=C/c1ccc(Cl)cc1)c1ccc(C(F)(F)F)cc1] | JN152 |
| 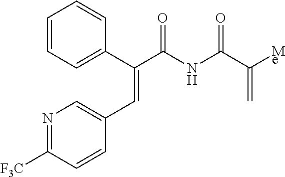 [SMILES: C=C(C)C(=O)NC(=O)/C(=C/c1ccc(C(F)(F)F)nc1)c1ccccc1] | JN153 |
| 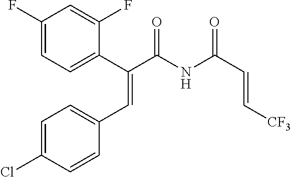 [SMILES: O=C(/C=C/C(F)(F)F)NC(=O)/C(=C/c1ccc(Cl)cc1)c1ccc(F)cc1F] | JN154 |
| 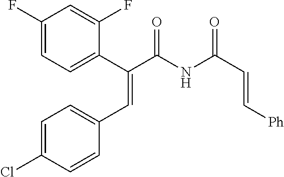 [SMILES: O=C(/C=C/c1ccccc1)NC(=O)/C(=C/c1ccc(Cl)cc1)c1ccc(F)cc1F] | JN155 |
| 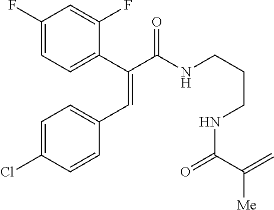 [SMILES: C=C(C)C(=O)NCCCNC(=O)/C(=C/c1ccc(Cl)cc1)c1ccc(F)cc1F] | JN156 |
| 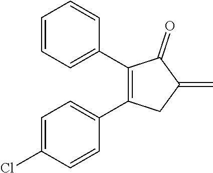 [SMILES: C=C1CC(c2ccc(Cl)cc2)=C(c2ccccc2)C1=O] | |
| 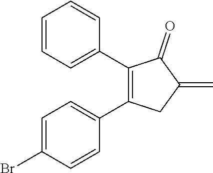 [SMILES: C=C1CC(c2ccc(Br)cc2)=C(c2ccccc2)C1=O] | |
| 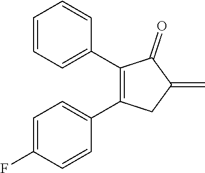 [SMILES: C=C1CC(c2ccc(F)cc2)=C(c2ccccc2)C1=O] | |
| 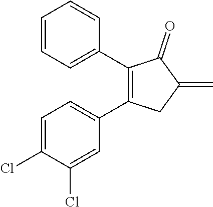 [SMILES: C=C1CC(c2ccc(Cl)c(Cl)c2)=C(c2ccccc2)C1=O] | |
| 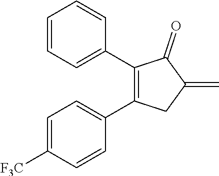 [SMILES: C=C1CC(c2ccc(C(F)(F)F)cc2)=C(c2ccccc2)C1=O] | |
| 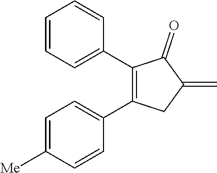 [SMILES: C=C1CC(c2ccc(C)cc2)=C(c2ccccc2)C1=O] | |
| 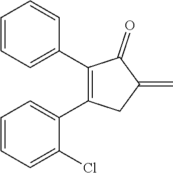 [SMILES: C=C1CC(c2ccccc2Cl)=C(c2ccccc2)C1=O] | |
| 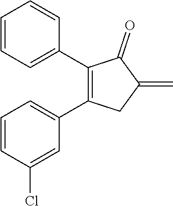 [SMILES: C=C1CC(c2cccc(Cl)c2)=C(c2ccccc2)C1=O] | |
| 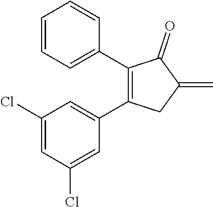 [SMILES: C=C1CC(c2cc(Cl)cc(Cl)c2)=C(c2ccccc2)C1=O] | |
| 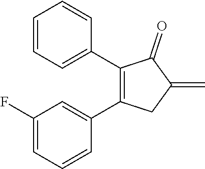 [SMILES: C=C1CC(c2cccc(F)c2)=C(c2ccccc2)C1=O] | |
| 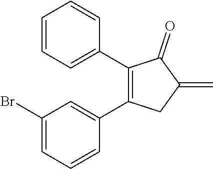 [SMILES: C=C1CC(c2cccc(Br)c2)=C(c2ccccc2)C1=O] | |
| 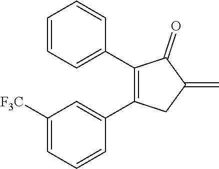 [SMILES: C=C1CC(c2cccc(C(F)(F)F)c2)=C(c2ccccc2)C1=O] | |
| 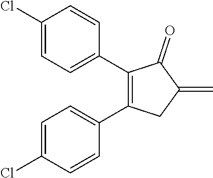 [SMILES: C=C1CC(c2ccc(Cl)cc2)=C(c2ccc(Cl)cc2)C1=O] | |
| 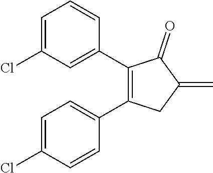 [SMILES: C=C1CC(c2ccc(Cl)cc2)=C(c2cccc(Cl)c2)C1=O] | |
| 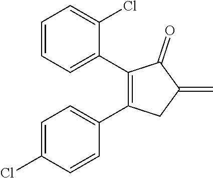 [SMILES: C=C1CC(c2ccc(Cl)cc2)=C(c2ccccc2Cl)C1=O] | |
| 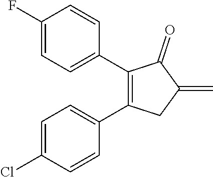 [SMILES: C=C1CC(c2ccc(Cl)cc2)=C(c2ccc(F)cc2)C1=O] | |
| 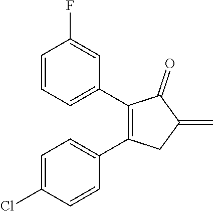 [SMILES: C=C1CC(c2ccc(Cl)cc2)=C(c2cccc(F)c2)C1=O] | |
| 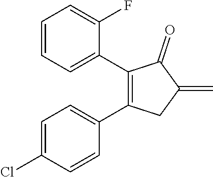 [SMILES: C=C1CC(c2ccc(Cl)cc2)=C(c2ccccc2F)C1=O] | |
| 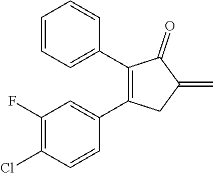 [SMILES: C=C1CC(c2ccc(Cl)c(F)c2)=C(c2ccccc2)C1=O] | |
| 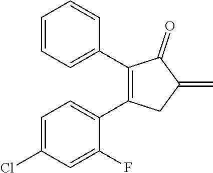 [SMILES: C=C1CC(c2ccc(Cl)cc2F)=C(c2ccccc2)C1=O] | |
| 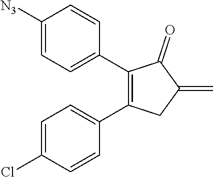 [SMILES: C=C1CC(c2ccc(Cl)cc2)=C(c2ccc(N=[N+]=[N-])cc2)C1=O] | |
| 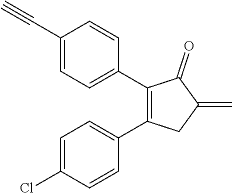 [SMILES: C#Cc1ccc(C2=C(c3ccc(Cl)cc3)CC(=C)C2=O)cc1] | |
| 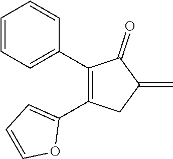 [SMILES: C=C1CC(c2ccco2)=C(c2ccccc2)C1=O] | |
| 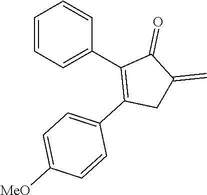 [SMILES: C=C1CC(c2ccc(OC)cc2)=C(c2ccccc2)C1=O] | JN053 |

The compounds disclosed herein are believed to AR degraders that directly target the TAD. By targeting the AR and its splice variants, these compounds offer the promise of overcoming AR-dependent castration resistance irrespective of the underlying molecular mechanism(s), including but not limited to the expression of constitutively active ARSVs that lack a functional C-terminal LBD.

In certain aspects, the present disclosure comprises a compound of the disclosure and a pharmaceutically acceptable excipient.

### EXAMPLES

The invention now being generally described, it will be more readily understood by reference to the following examples, which are included merely for purposes of illustration of certain aspects and embodiments of the present invention, and are not intended to limit the invention.

### Chemistry

### General Materials and Methods

All solvents and reagents were purchased from commercial sources and used without further purification unless otherwise noted. Dichloromethane (calcium hydride), diethyl ether (sodium), and tetrahydrofuran (sodium) used for the reactions were dried by distillation over the indicated drying agents. All reactions were performed under an inert atmosphere of dry argon and monitored by thin layer chromatography (TLC) on pre-coated EMD silica gel 60 F₂₅₄ TLC aluminum sheets and visualized with a UV lamp. Flash column chromatography was performed on SiliaFlash P60 (SiliCycle Inc.) silica gel (40-63 μm, 60 Å pore size). Preparative scale thin layer chromatography was performed on glass-backed 20×20 cm (1500 μm thickness) preparative TLC plates (Analtech, Z513040). NMR spectra were obtained on a Bruker AV500 instrument at the UCLA MIC Magnetic Resonance Laboratory. NMR data were analyzed using the MestReNova NMR software (Mestrelab Research S. L., version 11.0.2). Chemical shifts (δ) are expressed in ppm and are internally referenced for ¹H NMR (CHCl₃ 7.26 ppm, DMSO-d₆ 2.50 ppm) and ¹³C NMR (CDCl₃ 77.16 ppm, DMSO-d₆ 39.52 ppm). DART-MS spectra were collected on a Thermo Exactive Plus MSD (Thermo Scientific) equipped with an ID-CUBE ion source and a Vapur Interface (IonSense). Both the source and MSD were controlled by Excalibur, version 3.0. The analyte was spotted onto OpenSpot sampling cards (IonSense) using dichloromethane or chloroform as the solvent. Ionization was accomplished using He plasma with no additional ionization agents. Melting points were recorded on a Büchi® B-545 melting point apparatus. Analytical HPLC was performed on a 2.0×50 mm Waters Corp. 1.5 μm C₁₈ analytical HPLC column. A linear gradient of mobile phase was used over 5 min from 5-95% MeCN/water containing 0.2% HCOOH. The flow rate was 0.4 mL/min and the peaks were detected by a LCT-Premier ESI-TOF mass spectrometer in the positive ion mode.

### Synthesis

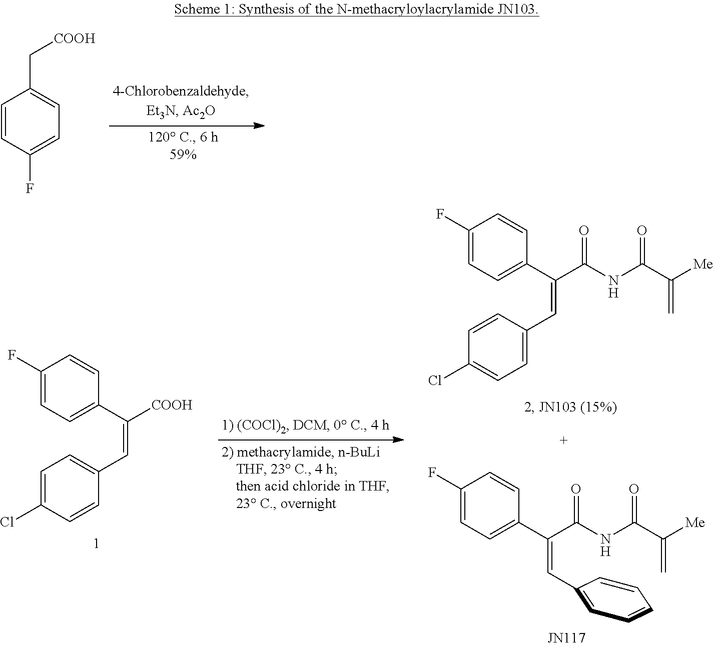

### (E)-3-(4-Chlorophenyl)-2-(4-fluorophenyl)acrylic Acid (1)

To 4-fluorophenylacetic acid (15.0 g, 95.4 mmol, 1.0 eq) and 4-chlorobenzaldehyde (13.61 g, 95.4 mmol, 1.0 eq) in a flask was added a mixture of acetic anhydride and triethylamine (v/v 1:1, 37.5 mL each). The resultant suspension was stirred at 120° C. for 6 h. Then it was cooled to 23° C. and 75 mL of conc. HCl and 225 mL of water were added whilst stirring. The flask was then left at 23° C. overnight, and the resultant precipitate filtered and washed with water. This crude product was recrystallized from ethanol/water (left at 23° C. overnight to complete precipitation) to yield acrylic acid 1 as a pale-brown solid (15.50 g, 56.0 mmol, 59%). ¹H NMR (500 MHz, DMSO-d₆) δ 12.84 (br s, 1H), 7.76 (s, 1H), 7.30 (d, J=8.6 Hz, 2H), 7.22-7.19 (m, 4H), 7.07 (d, J=8.6 Hz, 2H); ¹³C NMR (126 MHz, DMSO-d₆) δ 168.02, 161.67 (d, J=244.4 Hz), 138.07, 133.64, 133.30, 133.04, 131.76, 131.68 (d, J=8.2 Hz), 128.45, 128.14, 128.08, 115.54 (d, J=21.3 Hz).

### (E)-3-(4-Chlorophenyl)-2-(4-fluorophenyl)-N-methacryloylacrylamide (2, JN103)

The acrylic acid 1 (5.0 g, 18.1 mmol, 1.0 eq) was suspended in dichloromethane (75 mL) and the flask cooled to 0° C. To this was added oxalyl chloride (1.87 mL, 21.7 mmol, 1.2 eq) followed by anhydrous DMF (0.50 mL, slowly), and the solution left to stir at 0° C. for 4 h. Then the volatiles were removed in vacuo to yield the crude acid chloride as a brown waxy solid.

In a separate flask cooled in a Dry Ice-acetone bath, n-BuLi (7.20 mL of a 2.40 M solution in hexanes, 17.2 mmol, 0.95 eq) was added to a suspension of methacrylamide (1.49 g, 17.2 mmol, 0.95 eq) in tetrahydrofuran (100 mL), and stirring continued for further 4 h at 23° C. Then the acid chloride synthesized above was slowly added to the flask as a solution in tetrahydrofuran (25 mL). The resultant mixture was stirred overnight at 23° C., and then partitioned between EtOAc (200 mL) and saturated NH₄Cl/water (160:40 mL). The organic layer was separated and washed sequentially with saturated NaHCO₃/water (75:75 mL) and brine (100 mL). Then it was dried over anhydrous MgSO₄, filtered, and concentrated in vacuo. The crude residue was purified by column chromatography on silica gel using a mobile phase gradient of 0-20% EtOAc/hexanes, followed by a gradient of 15-20% EtOAc/hexanes containing 2% triethylamine additive. The isolated pale-yellow solid was then further purified by recrystallization in dichloromethane/hexanes to yield the N-methacryloylacrylamide 2 (JN103) as a white solid (953.6 mg, 2.8 mmol, 15%). Melting point 146.2-146.9° C.; ¹H NMR (500 MHz, DMSO-d₆) δ 10.56 (br s, 1H), 7.38 (s, 1H), 7.32 (d, J=8.6 Hz, 2H), 7.28-7.20 (m, 4H), 7.08 (d, J=8.6 Hz, 2H), 5.83 (s, 1H), 5.63 (q, J=1.5 Hz, 1H), 1.84 (t, J=1.2 Hz, 3H); ¹³C NMR (126 MHz, DMSO-d₆) δ 168.86, 167.99, 161.87 (d, J=245.4 Hz), 139.20, 136.05, 134.77, 133.44, 133.35, 131.75 (d, J=8.4 Hz), 131.50, 131.44 (d, J=3.4 Hz), 128.45, 123.05, 115.79 (d, J=21.5 Hz), 18.09; HRMS m/z calcd. for C₁₉H₁₆ClFNO₂ [M+H]⁺ 344.08481, found 344.08296; Analytical HPLC t_{R}=4.26 min.

### (Z)-3-(4-Chlorophenyl)-2-(4-fluorophenyl)-N-methacryloylacrylamide (JN117)

The Z-isomer (JN117) can be isolated from the same reaction chromatographed above to obtain JN103. Off-white solid. ¹H NMR (500 MHz, CDCl₃) δ 8.33 (br s, 1H), 7.48 (dd, J=8.9, 5.2 Hz, 2H), 7.35-7.29 (m, 4H), 7.08 (t, J=8.7 Hz, 2H), 6.88 (s, 1H), 5.48 (q, J=1.6 Hz, 1H), 5.46 (q, J=1.0 Hz, 1H), 1.83 (dd, J=1.6, 0.9 Hz, 3H). ¹³C NMR (126 MHz, CDCl₃) δ 170.12, 165.12, 163.07 (d, J=248.6 Hz), 139.29, 137.28, 134.50, 133.91, 132.42 (d, J=3.4 Hz), 129.69, 129.07, 128.62, 128.61 (d, J=8.1 Hz), 123.07, 115.96 (d, J=21.7 Hz), 18.22; HRMS m/z calcd. for C₁₉H₁₆ClFNO₂ [M+H]⁺ 344.08481, found 344.08448.

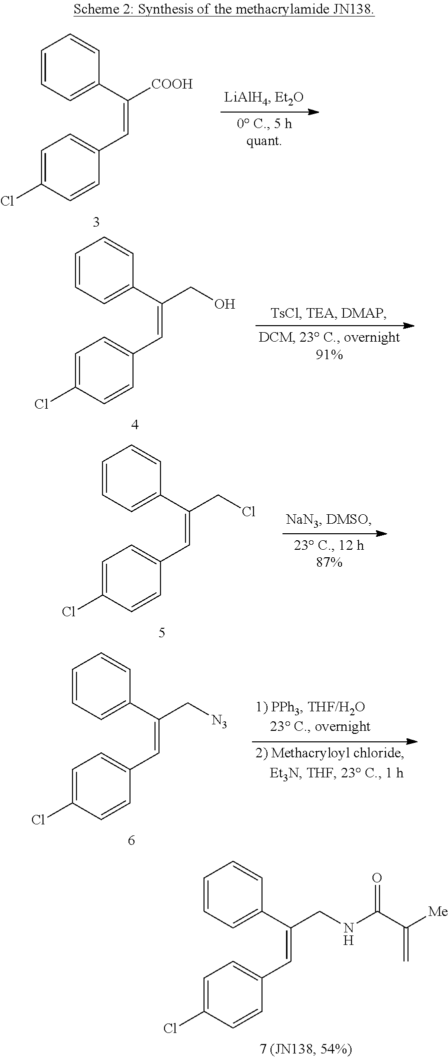 [SMILES: O=C(O)/C(=C/c1ccc(Cl)cc1)c1ccccc1 ; OC/C(=C/c1ccc(Cl)cc1)c1ccccc1 ; ClC/C(=C/c1ccc(Cl)cc1)c1ccccc1 ; [N-]=[N+]=NC/C(=C/c1ccc(Cl)cc1)c1ccccc1 ; C=C(C)C(=O)NC/C(=C/c1ccc(Cl)cc1)c1ccccc1]

### (E)-3-(4-Chlorophenyl)-2-phenylprop-2-en-1-ol (4)

To a solution of the acrylic acid 3 (5.1 g, 19.7 mmol, 1.0 eq) in diethyl ether (60 mL) at 0° C., was added lithium aluminum hydride (1.58 g, 39.4 mmol, 2.0 eq) in small portions. The resultant solution was stirred at 23° C. for 1.5 h and then quenched by the slow addition of water (8 mL). To this flask was added diethyl ether (50 mL), 15% NaOH solution (aq, 50 mL) and water (50 mL), and the solution stirred for 15 min at rt. It was then filtered through a plug of Celite, and the Celite washed with diethyl ether. Layers were separated in the filtrate, and the aqueous layer extracted with further diethyl ether (50 mL×2). The combined organic layers were washed with brine (150 mL), dried over anhydrous MgSO₄, filtered, and volatiles removed in vacuo to yield the α-hydroxy alkene 4 (4.81 g, 19.7 mmol, quant.) as a yellow oil. ¹H NMR (500 MHz, CDCl₃) δ 7.37-7.30 (m, 3H), 7.20 (dd, J=7.9, 1.7 Hz, 2H), 7.08 (d, J=8.5 Hz, 2H), 6.91 (d, J=8.6 Hz, 2H), 6.64 (d, J=1.5 Hz, 1H), 4.46 (d, J=1.5 Hz, 2H); ¹³C NMR (126 MHz, CDCl₃) δ 142.37, 138.24, 135.06, 132.59, 130.55, 129.08, 128.77, 128.29, 127.93, 125.21, 68.43.

### (E)-1-Chloro-4-(3-chloro-2-phenylprop-1-en-1-yl)benzene (5)

To a solution of the α-hydroxy alkene 4 (255.3 mg, 1.04 mmol, 1.0 eq) and triethylamine (0.43 mL, 3.1 mmol, 3.0 eq) in dichloromethane (8 mL) at 0° C. was added p-toluenesulfonyl chloride (242.8 mg, 1.25 mmol, 1.2 eq) and catalytic 4-dimethylaminopyridine (12.8 mg, 0.10 mmol, 0.10 eq). After the resultant solution stirred overnight at 23° C., the reaction mixture was diluted with EtOAc (40 mL) and washed with water (20 mL×2) and brine (20 mL). The resultant organic layer was dried over anhydrous MgSO₄, filtered, and concentrated in vacuo. The crude waxy residue was purified by column chromatography on silica gel using a mobile phase gradient of 0-3% EtOAc/hexanes to give the α-chloro alkene 5 as a colorless oil (249.4 mg, 0.95 mmol, 91%). ¹H NMR (500 MHz, CDCl₃) δ 7.41-7.30 (m, 3H), 7.23 (dd, J=7.6, 2.0 Hz, 2H), 7.09 (d, J=8.6 Hz, 2H), 6.90 (d, J=8.6 Hz, 2H), 6.74 (s, 1H), 4.43 (d, J=1.0 Hz, 2H); ¹³C NMR (126 MHz, CDCl₃) δ 138.61, 137.83, 134.40, 133.30, 130.68, 129.84, 129.03, 128.88, 128.38, 128.21, 51.39.

### (E)-1-(3-Azido-2-phenylprop-1-en-1-yl)-4-chlorobenzene (6)

The α-chloro alkene 5 (144.0 mg, 0.55 mmol, 1.0 eq) was dissolved in 3 mL of DMSO. To this was added a solution of sodium azide (106.7 mg, 1.6 mmol, 3.0 eq) in water (1 mL) and the resultant suspension left to stir overnight at 23° C. Then the reaction mixture was diluted with water (10 mL) and extracted with diethyl ether (8 mL×3). The combined organic layers were washed with water (10 mL) and brine (10 mL), dried over anhydrous MgSO₄, filtered, and concentrated in vacuo to yield the α-azido alkene 6 as a pale-yellow oil (129.1 mg, 0.48 mmol, 87%). ¹H NMR (500 MHz, CDCl₃) δ 7.39-7.32 (m, 3H), 7.21 (dd, J=7.7, 1.8 Hz, 2H), 7.09 (d, J=8.6 Hz, 2H), 6.92 (d, J=8.5 Hz, 2H), 6.63 (s, 1H), 4.15 (d, J=1.2 Hz, 2H); ¹³C NMR (126 MHz, CDCl₃) δ 138.14, 137.21, 134.45, 133.15, 130.69, 129.16, 128.81, 128.63, 128.39, 128.22, 59.05.

### (E)-N-(3-(4-Chlorophenyl)-2-phenylallyl)methacrylamide (7, JN138)

To the α-azido alkene 6 (118.7 mg, 0.44 mmol, 1.0 eq) in tetrahydrofuran/water (3 and 0.6 mL respectively) at 23° C. was added triphenylphosphine (256.5 mg, 0.97 mmol, 2.2 eq) and the resultant solution allowed to stir overnight. Then the reaction mixture was partitioned between EtOAc and water (10 mL each). The aqueous layer was extracted with further EtOAc (3 mL×2). The combined organic layers were washed with brine (10 mL), dried over anhydrous MgSO₄, filtered, and concentrated in vacuo. The crude α-amino alkene thus obtained was dissolved in tetrahydrofuran (3 mL) and the solution cooled to 0° C. To this was added triethylamine (0.12 mL, 0.88 mmol, 2.0 eq) and methacryloyl chloride (40 μL, 0.44 mmol, 1.0 eq). After stirring this mixture at 23° C. for 1 h, the contents were diluted with diethyl ether (8 mL) and washed with 0.1 N HCl (aq, 5 mL), water (2 mL), and saturated NaHCO₃(5 mL). The organic layer was then dried over anhydrous MgSO₄, filtered, and concentrated in vacuo. The crude residue was purified by preparative TLC on silica gel using a mobile phase of 70:30:2 hexanes/EtOAc/triethylamine to give methacrylamide 7 (JN138) as a white solid (74.1 mg, 0.24 mmol, 54%). ¹H NMR (500 MHz, CDCl₃) δ 7.38-7.28 (m, 3H), 7.18 (d, J=7.1 Hz, 2H), 7.06 (d, J=8.2 Hz, 2H), 6.88 (d, J=8.2 Hz, 2H), 6.54 (s, 1H), 5.98-5.83 (br m, 1H), 5.55 (s, 1H), 5.27 (s, 1H), 4.32 (d, J=5.9 Hz, 2H), 1.90 (s, 3H); ¹³C NMR (126 MHz, CDCl₃) δ 168.36, 140.14, 139.35, 138.36, 134.93, 132.65, 130.58, 129.09, 128.70, 128.26, 127.98, 126.53, 119.57, 47.24, 18.76.

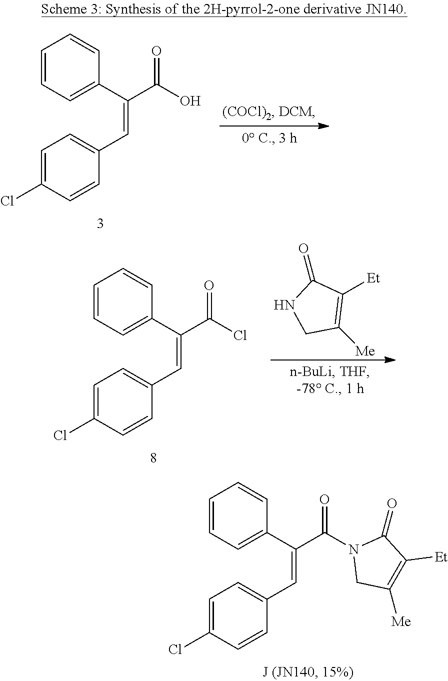 [SMILES: O=C(O)/C(=C/c1ccc(Cl)cc1)c1ccccc1 ; O=C(Cl)/C(=C/c1ccc(Cl)cc1)c1ccccc1 ; CCC1=C(C)CNC1=O ; CCC1=C(C)CN(C(=O)/C(=C/c2ccc(Cl)cc2)c2ccccc2)C1=O]

### (E)-1-(3-(4-Chlorophenyl)-2-phenylacryloyl)-3-ethyl-4-methyl-1,5-dihydro-2H-pyrrol-2-one (9, JN140)

Acrylic acid 3 (1.0 g, 3.87 mmol, 1.0 eq) was suspended in dichloromethane (16 mL) and the flask cooled to 0° C. To this was added oxalyl chloride (0.40 mL, 4.6 mmol, 1.2 eq) followed by anhydrous DMF (2 drops), and the solution left to stir at 0° C. for 3 h. Then the volatiles were removed in vacuo to yield the crude acid chloride 8 as a brown waxy solid, which was dissolved in to 10 mL of anhydrous tetrahydrofuran to make a ˜0.39 M solution of 8.

To 3-ethyl-4-methyl-1,5-dihydro-2H-pyrrol-2-one (140.5 mg, 1.10 mmol, 1.0 eq) at −78° C. in anhydrous tetrahydrofuran (6 mL) was added n-BuLi (0.45 mL of a 2.46 M solution in hexanes, 1.10 mmol, 1.0 eq), and the solution stirred for further 30 min. Then 2.82 mL (1.10 mmol, 1.0 eq) of the acid chloride (8) solution above was added. After stirring for another 1 h at −78° C., the reaction mixture was partitioned between EtOAc (10 mL) and saturated NH₄Cl/water (8:2 mL). The organic layer was washed with saturated NaHCO₃ (10 mL), dried over anhydrous MgSO₄, filtered, and concentrated in vacuo. The resultant crude material was purified by column chromatography on silica gel buffered with 2% triethylamine/hexanes using a mobile phase gradient of 0-20% EtOAc/hexanes to give the 2H-pyrrol-2-one 9 (JN140) as a pale-yellow wax (61.2 mg, 0.17 mmol, 15%). ¹H NMR (500 MHz, CDCl₃) δ 7.40-7.35 (m, 2H), 7.32-7.27 (m, 3H), 7.12 (d, J=8.6 Hz, 2H), 7.04 (d, J=8.5 Hz, 2H), 6.77 (s, 1H), 4.26 (q, J=1.0 Hz, 2H), 2.22 (q, J=7.6 Hz, 2H), 2.04 (t, J=1.0 Hz, 3H), 1.00 (t, J=7.6 Hz, 3H); ¹³C NMR (126 MHz, CDCl₃) δ 169.60, 169.36, 151.00, 137.86, 134.72, 134.21, 133.92, 133.81, 131.18, 131.07, 129.79, 128.56, 128.43, 128.24, 52.11, 16.81, 13.63, 12.92; HRMS m/z calcd. for C₂₂H₂₁ClNO₂ [M+H]⁺ 366.12553, found 366.12318.

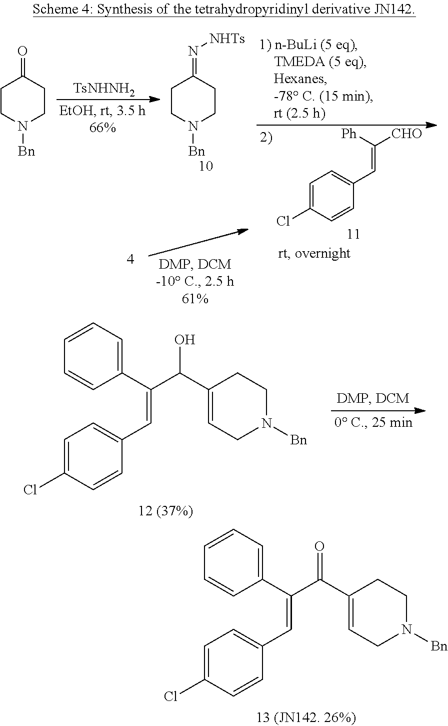

### N′-(1-Benzylpiperidin-4-ylidene)-4-methylbenzenesulfonohydrazide (10)

To tosylhydrazide (2.26 g, 11.7 mmol, 1.1 eq) in ethanol (25 mL) at 23° C. was added 1-benzylpiperidin-4-one (2.0 mL, 10.7 mmol, 1.0 eq), and the solution stirred for 3.5 h. The resultant solids were filtered, washed with ethanol, and dried in vacuo to yield the hydrazide derivative 10 (2.53 g, 7.1 mmol, 66%) as a white solid. ¹H NMR (500 MHz, DMSO-d₆) δ 10.20 (s, 1H), 7.71 (d, J=8.3 Hz, 2H), 7.38 (d, J=8.0 Hz, 2H), 7.35-7.27 (m, 4H), 7.27-7.21 (m, 1H), 3.48 (s, 2H), 2.45-2.31 (m, 9H), 2.17 (t, J=5.8 Hz, 2H); ¹³C NMR (126 MHz, DMSO-d₆) δ 159.26, 143.04, 138.27, 136.32, 129.37, 128.70, 128.19, 127.50, 126.95, 61.20, 53.03, 51.77, 33.94, 27.49, 21.01.

### (E)-3-(4-Chlorophenyl)-2-phenylacrylaldehyde (11)

To a cooled solution (ice-water bath) of the α-hydroxy alkene 4 (4.57 g, 18.7 mmol, 1.0 eq) dissolved in dichloromethane (90 mL) was added Dess-Martin periodinane (8.80 g, 20.5 mmol, 1.1 eq) in three portions. The resultant mixture was stirred at 4° C. for 2.5 h. Then 20 mL of saturated aq. NaHCO₃ solution was added to the flask and stirred for 5 min. The flask contents were then partitioned between further dichloromethane (60 mL) and saturated NaHCO₃ (aq, 80 mL). The organic layer was removed and washed with saturated NaHCO₃ (aq, 50 mL×3) and brine (50 mL). It was then dried over anhydrous MgSO₄, filtered, and concentrated in vacuo. The residue was purified by column chromatography on silica gel using a mobile phase gradient of 3-10% EtOAc/hexanes to give the enal 11 (2.79 g, 11.5 mmol, 61%) as a yellowish solid. ¹H NMR (500 MHz, CDCl₃) δ 9.77 (s, 1H), 7.44-7.38 (m, 3H), 7.34 (s, 1H), 7.20 (d, J=8.7 Hz, 2H), 7.19-7.16 (m, 2H), 7.13 (d, J=8.7 Hz, 2H); ¹³C NMR (126 MHz, CDCl₃) δ 193.77, 148.51, 142.27, 136.35, 133.08, 132.62, 131.99, 129.37, 129.14, 128.97, 128.68.

### (E)-1-(1-Benzyl-1,2,3,6-tetrahydropyridin-4-yl)-3-(4-chlorophenyl)-2-phenylprop-2-en-1-ol (12)

Tetramethylethylenediamine (0.21 mL, 1.4 mmol, 5.0 eq) was added to a cooled (−78° C.) solution of the hydrazide 10 (100.0 mg, 0.28 mmol, 1.0 eq) in hexanes (3 mL) and the solution stirred for 10 min. To this was added n-BuLi (0.57 mL of a 2.46 M solution in hexanes, 1.4 mmol, 5.0 eq), after which the solution was stirred for 15 min at −78° C. and 2.5 h at 23° C. The resultant solution was cooled in an ice-water bath, and the enal 11 (135.9 mg, 0.56 mmol, 2.0 eq) was added in a single portion. Then the reaction was allowed to warm to 23° C. and stir overnight, after which the reaction mixture was cooled (ice-water bath) and quenched by the addition of water (2 mL). The flask contents were then partitioned between diethyl ether (10 mL) and water (10 mL). The organic layer was washed with brine (10 mL), dried over anhydrous MgSO₄, filtered, and concentrated in vacuo. The crude mixture was purified by preparative TLC on silica gel using a mobile phase of 75:25:2 hexanes/EtOAc/triethylamine to give alcohol 12 as a yellow waxy residue (43.1 mg, 0.10 mmol, 37%). ¹H NMR (500 MHz, CDCl₃) δ 7.35-7.27 (m, 7H), 7.16-7.09 (m, 3H), 7.05 (d, J=8.6 Hz, 2H), 6.86 (d, J=8.6 Hz, 2H), 6.72-6.68 (m, 1H), 5.58-5.40 (m, 1H), 4.82 (s, 1H), 3.55 (s, 2H), 3.02-2.83 (m, 2H), 2.64-2.57 (m, 1H), 2.56-2.49 (m, 1H), 2.28-2.13 (m, 2H); ¹³C NMR (126 MHz, CDCl₃) δ 143.04, 138.25, 135.91, 135.15, 132.51, 130.61, 129.30, 129.25, 128.70, 128.36, 128.20, 127.64, 127.22, 126.25, 122.67, one sp2 peak overlapped, 79.81, 62.57, 52.57, 49.81, 25.26.

### (E)-1-(1-Benzyl-1,2,3,6-tetrahydropyridin-4-yl)-3-(4-chlorophenyl)-2-phenylprop-2-en-1-one (13, JN142)

To a cooled solution (ice-water bath) of the alcohol 12 (40.1 mg, 96.4 μmol, 1.0 eq) in dichloromethane (3 mL) was added Dess-Martin periodinane (51.6 mg, 160 μmol, 1.2 eq). The resultant mixture was stirred at 4° C. for 25 min. The flask contents were then partitioned between further dichloromethane (5 mL) and saturated NaHCO₃ (aq, 5 mL). The aqueous layer was extracted with further dichloromethane (3 mL). The combined organic layers were dried over anhydrous MgSO₄, filtered, and concentrated in vacuo. The crude mixture was purified by preparative TLC on silica gel using a mobile phase of 80:20:2 hexanes/EtOAc/triethylamine to give the tetrahydropyridinyl derivative 13 (JN142) as a yellow waxy residue (10.3 mg, 24.9 μmol, 26%). ¹H NMR (500 MHz, CDCl₃) δ 7.38-7.27 (m, 8H), 7.22-7.17 (m, 2H), 7.13 (d, J=8.6 Hz, 2H), 6.98 (d, J=8.5 Hz, 2H), 6.93 (s, 1H), 6.78 (tt, J=3.5, 1.5 Hz, 1H), 3.63 (s, 2H), 3.23-3.17 (m, 2H), 2.64 (t, J=5.7 Hz, 2H), 2.55-2.47 (m, 2H); ¹³C NMR (126 MHz, CDCl₃) δ 197.20, 141.23, 140.35, 137.86, 137.12, 136.40, 134.41, 134.31, 133.60, 131.30, 129.30, 129.26, 128.99, 128.58, 128.50, 128.16, 127.43, 62.67, 53.12, 49.49, 24.99; HRMS m/z calcd. for C₂₇H₂₅ClNO [M+H]⁺ 414.16192, found 414.16044.

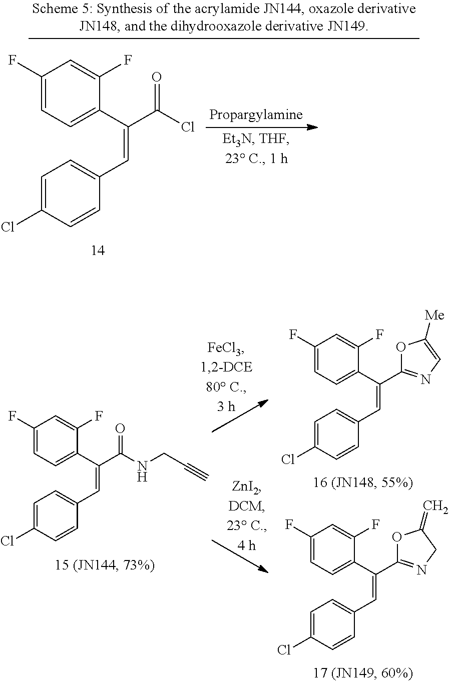 [SMILES: O=C(Cl)/C(=C/c1ccc(Cl)cc1)c1ccc(F)cc1F ; C#CCNC(=O)/C(=C/c1ccc(Cl)cc1)c1ccc(F)cc1F ; C=C1CN=C(/C(=C/c2ccc(Cl)cc2)c2ccc(F)cc2F)O1 ; Cc1cnc(/C(=C/c2ccc(Cl)cc2)c2ccc(F)cc2F)o1]

### (E)-3-(4-Chlorophenyl)-2-(2,4-difluorophenyl)-N-(prop-2-yn-1-yl)acrylamide (15, JN144)

To a solution of triethylamine (0.87 mL, 6.24 mmol, 3.0 eq) and propargylamine (0.41 mL, 6.24 mmol, 3.0 eq) in tetrahydrofuran (8 mL) at 0° C. was added the acid chloride 14 (4.0 mL of a 0.52 M solution, 2.08 mmol, 1.0 eq). The resultant solution was stirred for 1 h at 0° C. and then for 1 h at 23° C. The flask contents were then partitioned between EtOAc (30 mL) and saturated NH₄Cl/water (24:6 mL). The organic layer was washed with water (20 mL) and saturated NaHCO₃ (20 mL), dried over anhydrous MgSO₄, filtered, and concentrated in vacuo. The crude residue was purified by column chromatography on silica gel buffered with 2% triethylamine/hexanes using a mobile phase gradient of 0 to 30% EtOAc/hexanes to give the acrylamide 15 (JN144) as a white solid (502.1 mg, 1.51 mmol, 73%). ¹H NMR (500 MHz, CDCl₃) δ 7.90 (s, 1H), 7.23-7.13 (m, 3H), 7.02-6.91 (m, 4H), 5.59 (br t, J=5.5 Hz, 1H), 4.13 (dd, J=5.4, 2.6 Hz, 2H), 2.21 (t, J=2.6 Hz, 1H); ¹³C NMR (126 MHz, CDCl₃) δ 165.71, 163.69 (dd, J=253.1, 12.2 Hz), 160.38 (dd, J=251.4, 11.6 Hz), 139.33, 135.28, 133.02, 132.85 (dd, J=9.6, 4.2 Hz), 131.04, 128.89, 127.32, 118.88 (dd, J=16.6, 3.9 Hz), 113.06 (dd, J=21.1, 3.9 Hz), 105.58 (t, J=25.4 Hz), 79.30, 71.91, 30.07.

### (E)-2-(2-(4-Chlorophenyl)-1-(2,4-difluorophenyl)vinyl)-5-methyloxazole (16, JN148)

Iron(III) chloride (24.3 mg, 0.15 mmol, 0.5 eq) was added to a solution of the acrylamide 15 (100.0 mg, 0.30 mmol, 1.0 eq) in 1,2-dichloroethane (1.5 mL) at 23° C. The resultant mixture was heated at 80° C. for 3 h and then cooled to 23° C. The flask contents were then partitioned between dichloromethane (5 mL) and water (5 mL). The aqueous layer was extracted with further dichloromethane (2 mL×2). The combined organic layers were washed with brine (5 mL), dried over anhydrous MgSO₄, filtered, and concentrated in vacuo. The crude mixture was purified by preparative TLC on silica gel using a mobile phase of 75:25:2 hexanes/EtOAc/triethylamine to give the oxazole derivative 16 (JN148) as a pale-yellow solid (54.3 mg, 0.16 mmol, 55%). ¹H NMR (500 MHz, CDCl₃) δ 7.63 (s, 1H), 7.23 (td, J=8.3, 6.4 Hz, 1H), 7.17 (d, J=8.6 Hz, 2H), 7.00 (d, J=8.4 Hz, 2H), 6.96-6.88 (m, 2H), 6.79 (q, J=1.2 Hz, 1H), 2.36 (d, J=1.2 Hz, 3H); ¹³C NMR (126 MHz, CDCl₃) δ 163.35 (dd, J=250.9, 12.1 Hz), 161.12, 160.46 (dd, J=250.8, 12.3 Hz), 149.38, 134.39, 133.81, 132.78 (dd, J=9.4, 4.7 Hz), 132.67, 130.65, 128.80, 124.97, 122.86, 119.57 (dd, J=16.4, 4.2 Hz), 112.25 (dd, J=21.3, 3.8 Hz), 104.96 (t, J=25.5 Hz), 11.27; HRMS m/z calcd. for C₁₈H₁₃ClF₂NO [M+H]⁺ 332.06482, found 332.06348.

### (E)-2-(2-(4-Chlorophenyl)-1-(2,4-difluorophenyl)vinyl)-5-methylene-4,5-dihydrooxazole (17, JN149)

Diiodozinc (95.7 mg, 0.30 mmol, 1.0 eq) was added to a solution of the acrylamide 15 (100.0 mg, 0.30 mmol, 1.0 eq) in dichloromethane (1.5 mL), and the resultant mixture stirred at 23° C. for 3 h. The flask contents were then partitioned between dichloromethane (5 mL) and water (5 mL). The aqueous layer was extracted with further dichloromethane (2 mL×2). The combined organic layers were washed with brine (5 mL), dried over anhydrous MgSO₄, filtered, and concentrated in vacuo. The crude mixture was purified by preparative TLC on silica gel using a mobile phase of 80:20:2 hexanes/EtOAc/triethylamine to give the dihydrooxazole derivative 17 (JN149) as an off-white solid (59.3 mg, 0.18 mmol, 60%). ¹H NMR (500 MHz, CDCl₃) δ 7.64 (s, 1H), 7.23-7.16 (m, 3H), 7.00 (d, J=8.3 Hz, 2H), 6.96-6.85 (m, 2H), 4.79 (q, J=3.0 Hz, 1H), 4.63-4.55 (m, 2H), 4.33 (q, J=2.7 Hz, 1H); ¹³C NMR (126 MHz, CDCl₃) δ 164.29, 163.34 (dd, J=251.2, 12.0 Hz), 160.25 (dd, J=251.1, 12.6 Hz), 158.74, 138.31, 135.27, 133.14, 132.62 (dd, J=9.5, 4.7 Hz), 131.01, 128.90, 122.45, 119.29 (dd, J=16.5, 4.1 Hz), 112.21 (dd, J=21.2, 3.9 Hz), 104.93 (t, J=25.5 Hz), 83.81, 58.45; HRMS m/z calcd. for C₁₈H₁₃ClF₂NO [M+H]⁺ 332.06482, found 332.06337.

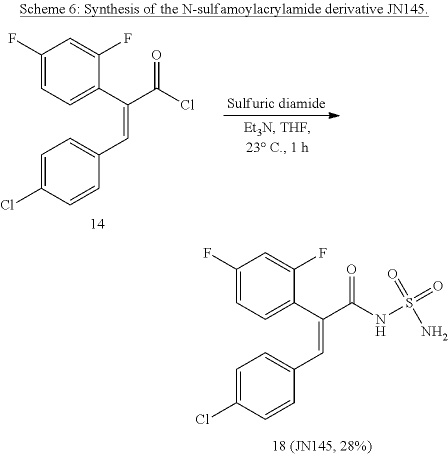 [SMILES: O=C(Cl)/C(=C/c1ccc(Cl)cc1)c1ccc(F)cc1F ; NS(=O)(=O)NC(=O)/C(=C/c1ccc(Cl)cc1)c1ccc(F)cc1F]

### (E)-3-(4-Chlorophenyl)-2-(2,4-difluorophenyl)-N-sulfamoylacrylamide (18, JN145)

To a stirred solution of triethylamine (0.87 mL, 6.24 mmol, 3.0 eq) and sulfuric diamide (833.0 mg, 8.32 mmol, 4.0 eq) in tetrahydrofuran (8 mL) at 0° C. was added the acid chloride 14 (4.0 mL of a 0.52 M solution, 2.08 mmol, 1.0 eq). The reaction was left to proceed for 1 h at 0° C. and then for 1 h at 23° C. The flask contents were then partitioned between EtOAc (5 mL) and saturated NH₄Cl/water (4:1 mL). The organic layer was separated and washed saturated NaHCO₃ (5 mL), dried over anhydrous MgSO₄, filtered, and concentrated in vacuo. The crude residue was purified by preparative TLC on silica gel using a mobile phase of 60:40:2 EtOAc/hexanes/triethylamine to give the N-sulfamoylacrylamide 18 (JN145) as a white solid (213.3 mg, 0.57 mmol, 28%). ¹H NMR (500 MHz, CDCl₃) δ 7.89 (s, 1H), 7.22 (td, J=8.3, 6.3 Hz, 1H), 7.17 (d, J=8.6 Hz, 2H), 7.02-6.91 (m, 4H), 5.79 (br s, 1H), 5.43 (br s, 1H); ³C NMR (126 MHz, CDCl₃) δ 167.94, 163.61 (dd, J=252.8, 11.8 Hz), 160.29 (dd, J=250.8, 11.5 Hz), 139.59, 135.38, 132.97, 132.70 (dd, J=9.5, 4.3 Hz), 131.10, 128.91, 127.30, 119.54 (dd, J=16.9, 4.0 Hz), 112.95 (dd, J=21.4, 3.8 Hz), 105.49 (t, J=25.3 Hz).

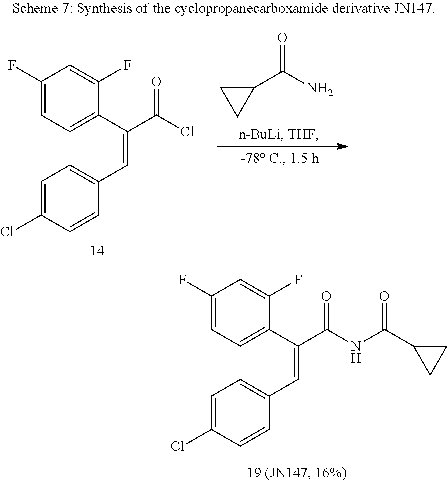 [SMILES: O=C(Cl)/C(=C/c1ccc(Cl)cc1)c1ccc(F)cc1F ; NC(=O)C1CC1 ; O=C(NC(=O)C1CC1)/C(=C/c1ccc(Cl)cc1)c1ccc(F)cc1F]

### (E)-N-(3-(4-Chlorophenyl)-2-(2,4-difluorophenyl)acryloyl)cyclopropanecarboxamide (19, JN147)

To a solution of cyclopropanecarboxamide (25.2 mg, 0.29 mmol, 0.90 eq) in tetrahydrofuran (3 mL) at −78° C. was added n-BuLi (0.12 mL of a 2.40 M solution in hexanes, 0.29 mmol, 0.90 eq) and the stirring continued for further 45 min at −78° C. Then the acid chloride 14 was slowly added to the flask as a solution in tetrahydrofuran (0.62 mL of a 0.52 M solution, 0.32 mmol, 1.0 eq). The resultant mixture was stirred further 1.5 h at −78° C., and the reaction quenched by the addition of 0.2 mL of saturated NH₄Cl solution. After allowing the reaction mixture to warm to 23° C., it was partitioned between EtOAc (5 mL) and saturated NH₄Cl/water (4:1 mL). The organic layer was separated and washed sequentially with saturated NH₄Cl/water (4:1 mL) and saturated NaHCO₃ (5 mL). Then it was dried over anhydrous MgSO₄, filtered, and concentrated in vacuo. The crude residue was purified by preparative TLC on silica gel using a mobile phase of 75:25:2 hexanes/EtOAc/triethylamine to give the cyclopropanecarboxamide derivative 19 (JN147) as an off-white solid (17.1 mg, 47.3 μmol, 16%). ¹H NMR (500 MHz, CDCl₃) δ 7.94 (s, 1H), 7.80 (br s, 1H), 7.23-7.16 (m, 3H), 7.05-6.93 (m, 4H), 3.05 (tt, J=7.9, 4.6 Hz, 1H), 1.15 (dt, J=4.8, 3.3 Hz, 2H), 1.04 (dt, J=8.3, 3.4 Hz, 2H); ¹³C NMR (126 MHz, CDCl₃) δ 176.74, 164.80, 164.05 (dd, J=253.5, 11.5 Hz), 160.46 (dd, J=251.7, 11.9 Hz), 141.86, 136.16, 132.84 (dd, J=9.7, 4.0 Hz), 132.47, 131.38, 129.10, 127.69, 117.94 (dd, J=16.7, 3.9 Hz), 113.44 (dd, J=21.5, 3.7 Hz), 105.88 (t, J=25.3 Hz), 14.62, 11.39.

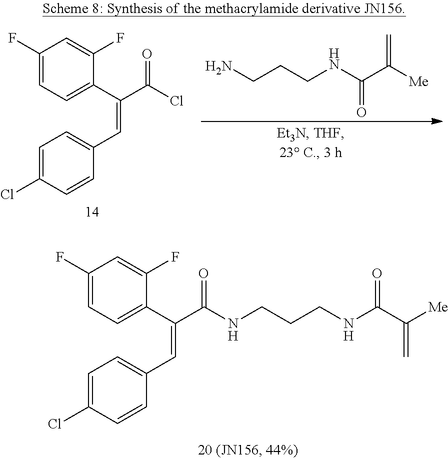

### (E)-3-(4-Chlorophenyl)-2-(2,4-difluorophenyl)-N-(3-methacrylamidopropyl)acrylamide (20, JN156)

A solution of triethylamine (0.31 mL, 1.6 mmol, 5.0 eq) and N-(3-aminopropyl)methacrylamide (90.3 mg, 0.48 mmol, 1.5 eq) in tetrahydrofuran (3 mL) was cooled in an ice-water bath, and the acid chloride 14 (0.62 mL of a 0.52 M solution, 0.32 mmol, 1.0 eq) was added. The reaction was left to proceed for 3 h at 23° C. and then the contents partitioned between EtOAc (5 mL) and saturated NH₄Cl/water (4:1 mL). The organic layer was separated and washed sequentially with saturated NH₄Cl/water (4:1 mL) and saturated NaHCO₃ (5 mL). Then it was dried over anhydrous MgSO₄, filtered, and concentrated in vacuo. The crude residue was purified by preparative TLC on silica gel using a mobile phase of 70:30:2 EtOAc/hexanes/triethylamine to give methacrylamide 20 (JN156) as an off-white solid (59.3 mg, 0.14 mmol, 44%). ¹H NMR (500 MHz, DMSO-d₆) δ 7.90 (br t, J=5.8 Hz, 1H), 7.75 (br t, J=5.9 Hz, 1H), 7.57 (s, 1H), 7.35-7.29 (m, 3H), 7.23 (td, J=8.5, 6.6 Hz, 1H), 7.13 (td, J=8.5, 2.6 Hz, 1H), 7.04 (d, J=8.7 Hz, 2H), 5.65-5.60 (m, 1H), 5.31 (p, J=1.6 Hz, 1H), 3.15 (q, J=6.8 Hz, 2H), 3.10 (q, J=6.8 Hz, 2H), 1.84 (t, J=1.2 Hz, 3H), 1.60 (p, J=6.9 Hz, 2H); ¹³C NMR (DMSO-d₆) δ 167.43, 166.07, 162.53 (dd, J=248.6, 13.6 Hz), 159.77 (dd, J=247.7, 13.0 Hz), 140.00, 135.26, 133.68, 133.24, 133.05 (dd, J=9.7, 4.6 Hz), 130.86, 130.30, 128.55, 119.69 (dd, J=16.6, 4.0 Hz), 118.85, 112.37 (dd, J=21.6, 3.4 Hz), 104.78 (t, J=26.0 Hz), 37.07, 36.40, 29.21, 18.62.

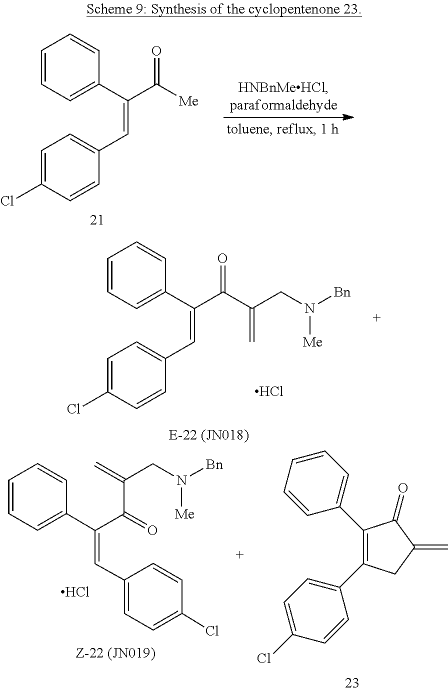 [SMILES: CC(=O)/C(=C/c1ccc(Cl)cc1)c1ccccc1 ; C=C(CN(C)Cc1ccccc1)C(=O)/C(=C/c1ccc(Cl)cc1)c1ccccc1 ; Cl ; C=C(CN(C)Cc1ccccc1)C(=O)/C(=C\c1ccc(Cl)cc1)c1ccccc1 ; Cl ; C=C1CC(c2ccc(Cl)cc2)=C(c2ccccc2)C1=O]

### 3-(4-Chlorophenyl)-5-methylene-2-phenylcyclopent-2-en-1-one (23)

The enone 21 (1.0 g, 3.9 mmol, 1.0 eq), paraformaldehyde (0.72 g, 23.4 mmol, 6.0 eq), and N-benzylmethylamine hydrochloride (1.36 g, 8.6 mmol, 2.2 eq) were dissolved in toluene (8 mL) and heated at reflux for 1 h. Then the reaction was quenched with the addition of 1 mL of 10% Na₂CO₃ (aq) while stirring. The solution was then partitioned between Et₂O (30 mL) and 10% Na₂CO₃ (aq, 30 mL). The layers were separated, and the aqueous layer was extracted with further Et₂O (10 mL×2). The combined organic layers were washed with brine (30 mL), dried over anhydrous MgSO₄, filtered, and concentrated in vacuo. The residue was purified by column chromatography on silica gel buffered with 1% triethylamine in hexanes using a mobile phase gradient of 3:100 to 15:100 mL of Et₂O/hexanes. The fractions containing 23 were further purified by preparative TLC on silica gel using a mobile phase of 15% EtOAc/hexanes to give cyclopentenone 23 as an off-white solid (10.4 mg, 37.0 μmol, 0.9%). R_{f} 0.16 (10% Et₂O/hexanes). ¹H NMR (500 MHz, CDCl₃) δ 7.39-7.32 (m, 3H), 7.31-7.22 (m, 7H), 6.31-6.26 (m, 1H), 5.61-5.57 (m, 1H), 3.97-3.21 (m, 2H); ¹³C NMR (126 MHz, CDCl₃) δ 194.08, 160.47, 141.99, 141.33, 136.20, 133.66, 132.27, 129.79, 129.51, 128.96, 128.77, 128.36, 117.62, 35.20; HRMS m/z calcd. for C₁₈H₁₄ClO [M+H]⁺ 281.07277, found 281.07161.

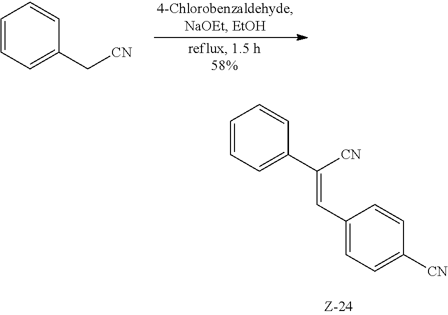 [SMILES: N#CCc1ccccc1 ; N#C/C(=C\c1ccc(C#N)cc1)c1ccccc1]

### (Z)-3-(4-Chlorophenyl)-2-phenylacrylonitrile (Z-24)

To a mixture of benzyl cyanide (10.0 mL, 84.9 mmol, 1.0 eq) and 4-chlorobenzaldehyde (12.1 g, 84.9 mmol, 1.0 eq) in absolute ethanol at 23° C. was added a freshly prepared solution of sodium ethoxide in ethanol (100 mL of a 1.27 M solution, 127.0 mmol, 1.5 eq). The resultant mixture was heated at reflux for 1.5 h, and then gradually cooled to 0° C. The resultant precipitate was filtered, washed with ice-cold absolute ethanol, and dried in vacuo to yield the acrylonitrile Z-24 (11.9 g, 49.6 mmol, 58%) as a white solid. ¹H NMR (400 MHz, CDCl₃) δ 7.83 (d, J=8.5 Hz, 2H), 7.70-7.65 (m, 2H), 7.50-7.40 (m, 6H); ¹³C NMR (101 MHz, CDCl₃) δ 140.76, 136.57, 134.28, 132.29, 130.60, 129.56, 129.38, 129.26, 126.13, 117.87, 112.43.

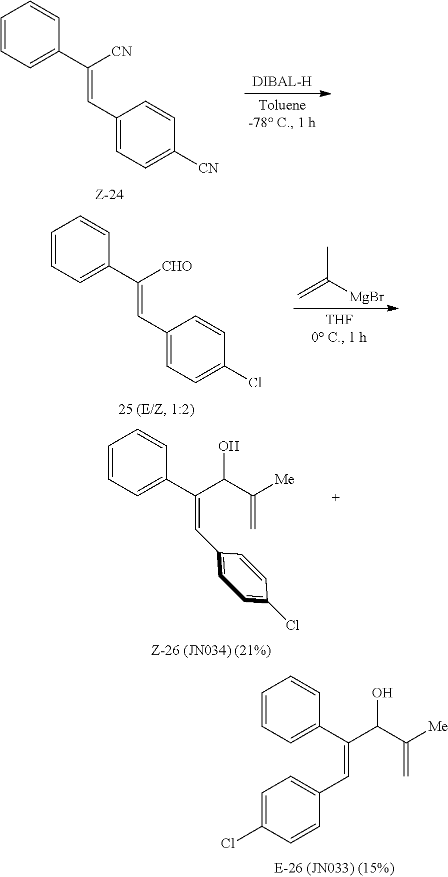

### 1-(4-Chlorophenyl)-4-methyl-2-phenylpenta-1,4-dien-3-ol (26; JN034 and JN033)

To a cooled (−78° C.) solution of the acrylonitrile Z-24 (2.0 g, 8.3 mmol, 1.0 eq) in toluene was added a 1.0 M solution of DIBAL-H (10.0 mL, 10.0 mmol, 1.2 eq). The resultant suspension was stirred for 1 h at −78° C. The reaction was allowed to warm to 0° C. and quenched by the addition of 5 mL of 5% H₂SO₄ (aq) at 0° C. To this was added a further 5% H₂SO₄ (aq, 45 mL) and Et₂O (50 mL), and the mixture stirred vigorously for 30 min at 0° C. After separating the layers, the aqueous layer was extracted with Et₂O (50 mL×2). The combined organic layers were washed with brine (75 mL), dried over anhydrous MgSO₄, filtered, and concentrated in vacuo. The crude enal 25 (2:1, E:Z) thus obtained was used for the next step without further purification.

A solution of the crude enal 25 above (8.3 mmol, 1.0 eq) in THF (40 mL) was cooled to 0° C. To this was added a solution of isopropenylmagnesium bromide (18.3 mL of a 0.50 M solution in THF, 9.1 mmol, 1.1 eq) and the reaction left to stir for 1 h at 0° C. To this mixture was added saturated NH₄Cl (aq, 5 mL) and the reaction contents partitioned between saturated NH₄Cl (aq, 50 mL), water (50 mL), and DCM (100 mL). The aqueous layer was further extracted with DCM (100 mL×2). The combined organic layers were washed with brine (150 mL), dried over anhydrous MgSO₄, filtered, and concentrated in vacuo. The crude material was purified by column chromatography on silica gel, using a mobile phase gradient of 0 to 10% of EtOAc/hexanes to yield the alcohols Z-26 (485.0 mg, 1.7 mmol, 21%) and E-26 (364.4 mg, 1.3 mmol, 15%) as pale-yellow oils.

Z-26: ¹H NMR δ 7.57-7.53 (m, 2H), 7.36-7.34 (m, 4H), 7.34-7.29 (m, 3H), 6.87 (s, 1H), 5.26 (d, J=5.6 Hz, 1H), 5.10 (s, 1H), 4.95 (q, J=1.6 Hz, 1H), 1.89 (d, J=5.6 Hz, 1H), 1.63 (d, J=1.4 Hz, 3H); ¹³C NMR δ 145.63, 142.52, 139.65, 135.44, 133.31, 131.82, 130.31, 128.74, 128.33, 128.21, 127.77, 111.38, 73.15, 20.10; HRMS m/z calcd. for C₁₈H₁₆Cl [M-OH]⁺ 267.09350, found 267.09213.

E-26: ¹H NMR (500 MHz, CDCl₃) δ 7.33-7.28 (m, 3H), 7.15-7.11 (m, 2H), 7.06 (d, J=8.6 Hz, 2H), 6.87 (d, J=8.7 Hz, 2H), 6.71 (s, 1H), 4.91 (s, 2H), 4.89 (d, J=4.8 Hz, 1H), 1.86 (d, J=4.4 Hz, 1H), 1.78 (s, 3H); ¹³C NMR (126 MHz, CDCl₃) δ 144.51, 142.89, 137.97, 135.11, 132.63, 130.65, 129.24, 128.77, 128.25, 127.77, 126.62, 113.26, 80.62, 18.43; HRMS m/z calcd. for C₁₈H₁₆Cl [M-OH]⁺ 267.09350, found 267.09195.

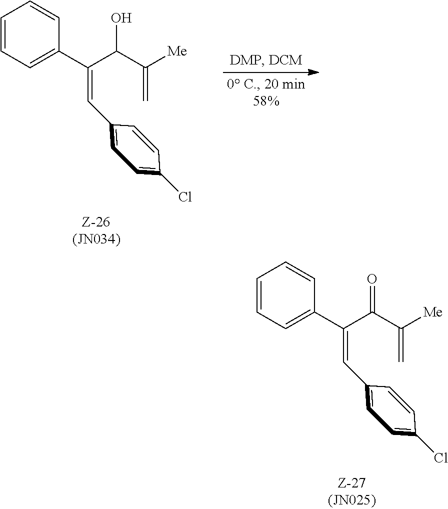

### (Z)-1-(4-Chlorophenyl)-4-methyl-2-phenylpenta-1,4-dien-3-one (Z-27)

A solution of the alcohol Z-26 (450.9 mg, 1.58 mmol, 1.0 eq) in DCM (10 mL) was cooled in an ice-water bath. To this was added Dess-Martin periodinane (738.7 mg, 1.74 mmol, 1.1 eq) and the reaction left to stir for 20 min at 0° C. To this mixture was added a saturated NaHCO₃ (aq, 3 mL) and the mixture stirred for 5 min. The contents were then partitioned between DCM (40 mL) and saturated NaHCO₃ (aq, 50 mL), and the layers were separated. The aqueous layer was extracted with further DCM (20 mL×2). The combined organic layers were dried over anhydrous MgSO₄, filtered, and concentrated in vacuo. The crude material was purified by column chromatography on silica gel, using a mobile phase gradient of 0 to 3% of EtOAc/hexanes to yield the dienone Z-27 (258.3 mg, 0.91 mmol, 58%) as a pale-yellow wax. ¹H NMR δ 7.42-7.29 (m, 5H), 7.26 (d, J=8.5 Hz, 2H), 7.18 (d, J=8.5 Hz, 2H), 7.02 (s, 1H), 5.99 (s, 1H), 5.81 (s, 1H), 1.94 (s, 3H); ¹³C NMR δ 201.53, 144.39, 141.85, 138.18, 134.58, 133.96, 130.30, 129.95, 128.99, 128.86, 128.49, 128.38, 126.31, 16.96; HRMS m/z calcd. for C₁₈H₁₆ClO [M+H]⁺ 283.08842, found 283.08642.

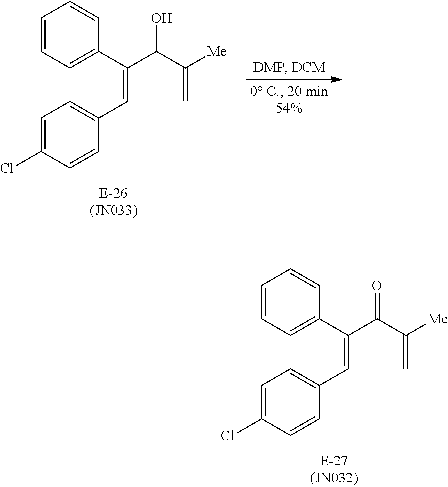 [SMILES: C=C(C)C(O)/C(=C/c1ccc(Cl)cc1)c1ccccc1 ; C=C(C)C(=O)/C(=C/c1ccc(Cl)cc1)c1ccccc1]

### (E)-1-(4-Chlorophenyl)-4-methyl-2-phenylpenta-1,4-dien-3-one (E-27)

Using the same procedure outlined for Z-27 above, the isomer E-27 was obtained as a white solid (54%). ¹H NMR δ 7.37-7.31 (m, 3H), 7.21-7.17 (m, 2H), 7.14 (d, J=8.6 Hz, 2H), 7.11 (s, 1H), 6.99 (d, J=8.3 Hz, 2H), 5.84 (p, J=1.0 Hz, 1H), 5.81 (p, J=1.5 Hz, 1H), 2.00 (dd, J=1.5, 0.9 Hz, 3H); ¹³C NMR δ 199.00, 144.31, 141.27, 136.37, 136.27, 134.63, 133.50, 131.51, 129.40, 129.01, 128.63, 128.20, 126.40, 18.76; HRMS m/z calcd. for C₁₈H₁₆ClO [M+H]⁺ 283.08842, found 283.08634.

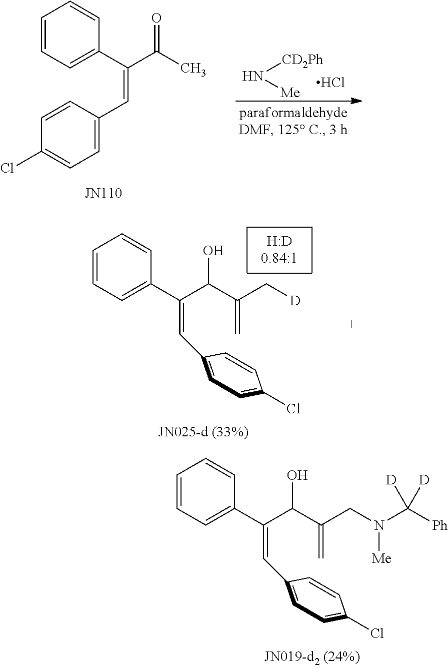

### (Z)-1-(4-Chlorophenyl)-4-(methyl-d)-2-phenylpenta-1,4-dien-3-one (JN025-d, H:D 0.84:1 Mixture)

The enone JN110 (77.0 mg, 0.30 mmol, 1.0 eq), paraformaldehyde (30.3 mg, 0.98 mmol, 3.3 eq), and N-methyl-1-phenylmethan-d₂-amine hydrochloride² (100.0 mg, 0.63 mmol, 2.1 eq) were dissolved in dimethylformamide (1 mL) and heated at 125° C. for 3 h. Then the volatiles were removed in vacuo and the remaining contents partitioned between Et₂O (7 mL) and 10% Na₂CO₃ (aq, 7 mL). The layers were separated, and the aqueous layer was extracted with further Et₂O (5 mL×2). The combined organic layers were washed with brine (5 mL), dried over anhydrous MgSO₄, filtered, and concentrated in vacuo. The residue was purified by column chromatography on silica gel buffered with 1% triethylamine in hexanes, using a mobile phase gradient of 3:100 to 15:100 mL of Et₂O/hexanes to yield JN025-d (H:D 0.84:1 mixture) as a yellow-colored wax (28.4 mg, 0.10 mmol, 33%). ¹H NMR (500 MHz, CDCl₃) δ 7.43-7.31 (m, 5H), 7.26 (d, J=8.5 Hz, 2H), 7.18 (d, J=8.4 Hz, 2H), 7.02 (s, 1H), 5.99 (t, J=0.9 Hz, 1H), 5.83-5.80 (m, 1H), 1.95 (s, 1.34H, —CH₃), 1.94-1.92 (br m, 1.07H, —CH₂D); ²H NMR (77 MHz, CDCl₃) δ 1.94 (t, J=2.2 Hz); ¹³C NMR (126 MHz, CDCl₃) δ 201.53, 201.51, 144.39, 144.36, 141.85, 138.18, 134.58, 133.96, 130.30, 129.95, 128.99, 128.86, 128.49, 128.38, 126.31, 16.97 (CH₃), 16.72 (t, J=19.7 Hz, —CH₂D); HRMS m/z calcd. for C₁₈H₁₅DClO [M+H]⁺ 284.09470, found 284.09347; HRMS m/z calcd. for C₁₈H₁₆ClO [M+H]⁺ 283.08842, found 283.08734.

### (Z)-1-(4-Chlorophenyl)-4-((methyl(phenylmethyl-d₂)amino)methyl)-2-phenylpenta-1,4-dien-3-one (JN019-d₂)

From the same reaction (above) that yielded JN025-d, compound JN019-d₂ was isolated as a pale-yellow wax (28.8 mg, 71.3 μmol, 24%). ¹H NMR (400 MHz, CDCl₃) δ 7.43-7.38 (m, 2H), 7.38-7.32 (m, 3H), 7.32-7.27 (m, 5H), 7.20 (s, 4H), 7.02 (s, 1H), 6.19 (q, J=1.2 Hz, 1H), 6.11 (q, J=1.5 Hz, 1H), 3.29 (s, 2H), 2.06 (s, 3H); ²H NMR (77 MHz, CDCl₃) δ 3.46 (s); ¹³C NMR (126 MHz, CDCl₃) δ 200.90, 145.47, 141.87, 138.15, 134.46, 133.95, 130.99, 130.10, 128.95, 128.84, 128.82, 128.56, 128.47, 128.35, 128.33, 127.11, 126.41, 61.71 (weak p, J=19.2 Hz), 56.21, 42.24; HRMS m/z calcd. for C₂₆H₂₃D₂ClNO [M+H]⁺ 404.17447, found 404.17404.

### Characterization

| Compound | | Formula² | |
|---|---|---|---|
| No. | NMR¹ | m/z (calc.) | m/z (meas.) |
| JN103 | ¹H NMR (DMSO-d₆) δ 10.56 (br s, 1H), 7.38 | C₁₉H₁₆ClFNO₂ | 344.08296 |
| | (s, 1H), 7.32 (d, J = 8.6 Hz, 2H), 7.28-7.20 | 344.08481 | |
| | (m, 4H), 7.08 (d, J = 8.6 Hz, 2H), 5.83 (s, | | |
| | 1H), 5.63 (q, J = 1.5 Hz, 1H), 1.84 (t, J = 1.2 | | |
| | Hz, 3H); ¹³C NMR (DMSO-d₆) δ 168.86, | | |
| | 167.99, 161.87 (d, J = 245.4 Hz), 139.20, | | |
| | 136.05, 134.77, 133.44, 133.35, 131.75 (d, | | |
| | J = 8.4 Hz), 131.50, 131.44 (d, J = 3.4 Hz), | | |
| | 128.45, 123.05, 115.79 (d, J = 21.5 Hz), | | |
| | 18.09. | | |
| JN138 | ¹H NMR δ 7.38-7.28 (m, 3H), 7.18 (d, J = | C₁₉H₁₆ClNO | 312.11405 |
| | 7.1 Hz, 2H), 7.06 (d, J = 8.2 Hz, 2H), 6.88 | 312.11497 | |
| | (d, J = 8.2 Hz, 2H), 6.54 (s, 1H), 5.98-5.83 | | |
| | (br m, 1H), 5.55 (s, 1H), 5.27 (s, 1H), 4.32 | | |
| | (d, J = 5.9 Hz, 2H), 1.90 (s, 3H); ¹³C NMR δ | | |
| | 168.36, 140.14, 139.35, 138.36, 134.93, | | |
| | 132.65, 130.58, 129.09, 128.70, 128.26, | | |
| | 127.98, 126.53, 119.57, 47.24, 18.76. | | |
| JN139 | ¹H NMR δ 7.88 (s, 1H), 7.74 (br s, 1H), 7.32 | C₁₈H₁₄ClFNO₂ | 330.06919 |
| | (dd, J = 17.1, 10.4 Hz, 1H), 7.25-7.15 (m, | 330.06916 | |
| | 6H), 6.93 (d, J = 8.6 Hz, 2H), 6.49 (dd, J = | | |
| | 17.1, 1.7 Hz, 1H), 5.91 (dd, J = 10.4, 1.4 Hz, | | |
| | 1H); ¹³C NMR δ 166.80, 165.08, 163.41 | | |
| | (d, J = 250.9 Hz), 140.43, 135.98, 133.13, | | |
| | 132.52, 131.98 (2C), 131.91 (d, J = 8.2 Hz), | | |
| | 130.09 (d, J = 3.7 Hz), 129.75, 128.95, 117.75 | | |
| | (d, J = 21.7 Hz). | | |
| JN140 | ¹H NMR δ 7.40-7.35 (m, 2H), 7.32-7.27 | C₂₂H₂₁ClNO₂ | 366.12318 |
| | (m, 3H), 7.12 (d, J = 8.6 Hz, 2H), 7.04 | 366.12553 | |
| | (d, J = 8.5 Hz, 2H), 6.77 (s, 1H), 4.26 | | |
| | (q, J = 1.0 Hz, 2H), 2.22 (q, J = 7.6 Hz, 2H), | | |
| | 2.04 (t, J = 1.0 Hz, 3H), 1.00 (t, J = 7.6 Hz, 3H); | | |
| | ¹³C NMR δ 169.60, 169.36, 151.00, 137.86, | | |
| | 134.72, 134.21, 133.92, 133.81, 131.18, | | |
| | 131.07, 129.79, 128.56, 128.43, 128.24, | | |
| | 52.11, 16.81, 13.63, 12.92. | | |
| JN141 | ¹H NMR δ 7.35-7.27 (m, 5H), 7.13 (d, J = | C₁₉H₁₇ClNO₂ | 326.09271 |
| | 8.5 Hz, 2H), 7.01 (d, J = 8.5 Hz, 2H), 6.79 | 326.09423 | |
| | (s, 1H), 3.89-3.84 (m, 2H), 2.49 (t, J = 8.0 | | |
| | Hz, 2H), 2.10-2.00 (m, 2H); ¹³C NMR δ | | |
| | 174.07, 170.98, 138.12, 134.64, 134.02, | | |
| | 133.75, 131.92, 131.10, 129.80, 128.52, | | |
| | 128.46, 128.33, 45.97, 33.08, 17.66. | | |
| JN142 | ¹H NMR δ 7.38-7.27 (m, 8H), 7.22-7.17 | C₂₇H₂₅ClNO | 414.16044 |
| | (m, 2H), 7.13 (d, J = 8.6 Hz, 2H), 6.98 (d, J = | 414.16192 | |
| | 8.5 Hz, 2H), 6.93 (s, 1H), 6.78 (tt, J = 3.5, | | |
| | 1.5 Hz, 1H), 3.63 (s, 2H), 3.23-3.17 (m, | | |
| | 2H), 2.64 (t, J = 5.7 Hz, 2H), 2.55-2.47 (m, | | |
| | 2H); ¹³C NMR δ 197.20, 141.23, 140.35, | | |
| | 137.86, 137.12, 136.40, 134.41, 134.31, | | |
| | 133.60, 131.30, 129.30, 129.26, 128.99, | | |
| | 128.58, 128.50, 128.16, 127.43, 62.67, | | |
| | 53.12, 49.49, 24.99. | | |
| JN143 | ¹H NMR δ 7.28-7.22 (m, 1H), 7.18 (d, J = | C₂₀H₁₅ClF₂NO₃ | 390.06898 |
| | 8.5 Hz, 2H), 7.12 (s, 1H), 7.06 (d, J = 8.4 | 390.07030 | |
| | Hz, 2H), 6.82-6.76 (m, 2H), 4.99 (d, J = 0.9 | | |
| | Hz, 1H), 4.38 (d, J = 0.9 Hz, 2H), 3.86 (s, | | |
| | 3H); ¹³C NMR δ 176.25, 168.72, 168.68, | | |
| | 163.07 (dd, J = 250.6, 11.8 Hz), 160.37 (dd, | | |
| | J = 251.7, 11.9 Hz), 136.86, 134.73, 133.58, | | |
| | 133.49 (dd, J = 9.5, 4.8 Hz), 130.90, 130.81, | | |
| | 128.70, 119.30 (dd, J = 15.0, 4.1 Hz), 111.54 | | |
| | (dd, J = 21.4, 3.5 Hz), 104.40 (t, J = 25.5 | | |
| | Hz), 94.29, 58.89, 48.81. | | |
| JN144 | ¹H NMR δ 7.90 (s, 1H), 7.23-7.13 (m, 3H), | C₁₈H₁₃ClF₂NO | 332.06397 |
| | 7.02-6.91 (m, 4H), 5.59 (br t, J = 5.5 Hz, | 332.06482 | |
| | 1H), 4.13 (dd, J = 5.4, 2.6 Hz, 2H), 2.21 | | |
| | (t, J = 2.6 Hz, 1H); ¹³C NMR δ 165.71, 163.69 | | |
| | (dd, J = 253.1, 12.2 Hz), 160.38 (dd, J = | | |
| | 251.4, 11.6 Hz), 139.33, 135.28, 133.02, | | |
| | 132.85 (dd, J = 9.6, 4.2 Hz), 131.04, 128.89, | | |
| | 127.32, 118.88 (dd, J = 16.6, 3.9 Hz), 113.06 | | |
| | (dd, J = 21.1, 3.9 Hz), 105.58 (t, J = 25.4 | | |
| | Hz), 79.30, 71.91, 30.07. | | |
| JN145 | ¹H NMR δ 7.89 (s, 1H), 7.22 (td, J = 8.3, 6.3 | [M − H]⁻ | 371.02516 |
| | Hz, 1H), 7.17 (d, J = 8.6 Hz, 2H), 7.02-6.91 | C₁₅H₁₀ClF₂N₂O₃S | |
| | (m, 4H), 5.79 (br s, 1H), 5.43 (br s, 1H); | 371.00742 | |
| | ¹³C NMR δ 167.94, 163.61 (dd, J = 252.8, | | |
| | 11.8 Hz), 160.29 (dd, J = 250.8, 11.5 Hz), | | |
| | 139.59, 135.38, 132.97, 132.70 (dd, J = 9.5, | | |
| | 4.3 Hz), 131.10, 128.91, 127.30, 119.54 (dd, | | |
| | J = 16.9, 4.0 Hz), 112.95 (dd, J = 21.4, 3.8 | | |
| | Hz), 105.49 (t, J = 25.3 Hz). | | |
| JN146 | ¹H NMR (DMSO-d₆) δ 8.81 (s, 1H), 7.57 | C₁₉H₁₄ClF₂N₂O | 359.07483 |
| | (s, 1H), 7.36-7.29 (m,3 H), 7.25-7.17 (m, 1H), | 359.07572 | |
| | 7.11 (td, J = 8.6, 2.3 Hz, 1H), 7.06 (d, J = | | |
| | 8.6 Hz, 2H), 1.51 (dd, J = 8.4, 5.7 Hz, 2H), | | |
| | 1.18 (dd, J = 8.2, 5.6 Hz, 2H); ¹³C NMR | | |
| | (DMSO-d₆) δ 167.69, 162.59 (dd, J = 247.9, | | |
| | 12.5 Hz), 159.82 (dd, J = 248.4, 13.3 Hz), | | |
| | 136.68, 133.61, 133.30, 133.17 (dd, J = 9.9, | | |
| | 4.7 Hz), 131.00, 129.13, 128.63, 120.84, | | |
| | 119.21 (dd, J = 16.4, 3.8 Hz), 112.30 (dd, J = | | |
| | 21.2, 3.6 Hz), 104.72 (t, J = 26.1 Hz), 20.50, | | |
| | 15.85. | | |
| JN147 | ¹H NMR δ 7.94 (s, 1H), 7.80 (br s, 1H), | C₁₉H₁₅ClF₂NO₂ | 362.07421 |
| | 7.23-7.16 (m, 3H), 7.05-6.93 (m, 4H), 3.05 | 362.07539 | |
| | (tt, J = 7.9, 4.6 Hz, 1H), 1.15 (dt, J = 4.8, | | |
| | 3.3 Hz, 2H), 1.04 (dt, J = 8.3, 3.4 Hz, 2H); | | |
| | ¹³C NMR δ 176.74, 164.80, 164.05 (dd, J = | | |
| | 253.5, 11.5 Hz), 160.46 (dd, J = 251.7, 11.9 | | |
| | Hz), 141.86, 136.16, 132.84 (dd, J = 9.7, 4.0 | | |
| | Hz), 132.47, 131.38, 129.10, 127.69, 117.94 | | |
| | (dd, J = 16.7, 3.9 Hz), 113.44 (dd, J = 21.5, | | |
| | 3.7 Hz), 105.88 (t, J = 25.3 Hz), 14.62, 11.39. | | |
| JN148 | ¹H NMR δ 7.63 (s, 1H), 7.23 (td, J = 8.3, 6.4 | C₁₈H₁₃ClF₂NO | 332.06348 |
| | Hz, 1H), 7.17 (d, J = 8.6 Hz, 2H), 7.00 (d, J = | 332.06482 | |
| | 8.4 Hz, 2H), 6.96-6.88 (m, 2H), 6.79 (q, J = | | |
| | 1.2 Hz, 1H), 2.36 (d, J = 1.2 Hz, 3H); | | |
| | ¹³C NMR δ 163.35 (dd, J = 250.9, 12.1 Hz), | | |
| | 161.12, 160.46 (dd, J = 250.8, 12.3 Hz), | | |
| | 149.38, 134.39, 133.81, 132.78 (dd, J = 9.4, | | |
| | 4.7 Hz), 132.67, 130.65, 128.80, 124.97, | | |
| | 122.86, 119.57 (dd, J = 16.4, 4.2 Hz), 112.25 | | |
| | (dd, J = 21.3, 3.8 Hz), 104.96 (t, J = 25.5 | | |
| | Hz), 11.27. | | |
| JN149 | ¹H NMR δ 7.64 (s, 1H), 7.23-7.16 (m, 3H), | C₁₈H₁₃ClF₂NO | 332.06337 |
| | 7.00 (d, J = 8.3 Hz, 2H), 6.96-6.85 (m, 2H), | 332.06482 | |
| | 4.79 (q, J = 3.0 Hz, 1H), 4.63-4.55 (m, 2H), | | |
| | 4.33 (q, J = 2.7 Hz, 1H); ¹³C NMR δ 164.29, | | |
| | 163.34 (dd, J = 251.2, 12.0 Hz), 160.25 (dd, | | |
| | J = 251.1, 12.6 Hz), 158.74, 138.31, 135.27, | | |
| | 133.14, 132.62 (dd, J = 9.5, 4.7 Hz), 131.01, | | |
| | 128.90, 122.45, 119.29 (dd, J = 16.5, 4.1 | | |
| | Hz), 112.21 (dd, J = 21.2, 3.9 Hz), 104.93 (t, | | |
| | J = 25.5 Hz), 83.81, 58.45. | | |
| JN150 | ¹H NMR δ 8.02 (br s, 1H), 7.82 (s, 1H), | C₂₅H₁₉ClF₂NO₂ | 438.10584 |
| | 7.31-7.25 (m, 2H), 7.24-7.09 (m, 3H), 7.17 | 438.10669 | |
| | (m, 3H), 7.16-7.05 (m, 2H), 6.98-6.91 (m, 4H), | | |
| | 5.59 (s,1H), 5.35 (t, J = 1.5 Hz, 1H), 3.61 (s, 2H); | | |
| | ¹³C NMR δ 166.03, 164.12, 163.87 (dd, J = | | |
| | 253.4, 11.6 Hz), 160.32 (dd, J = 251.7, 11.9 | | |
| | Hz), 143.93, 141.92, 137.47, 136.08, 132.91 | | |
| | (dd, J = 9.6, 4.1 Hz), 132.47, 131.35, 129.04, | | |
| | 129.02, 128.85, 127.50, 126.94, 122.89, | | |
| | 118.14 (dd, J = 16.5, 4.0 Hz), 113.29 (dd, J = | | |
| | 21.4, 3.8 Hz), 105.66 (t, J = 25.3 Hz), 38.05. | | |
| JN151 | ¹H NMR δ 7.91 (s, 1H), 7.19 (d, J = 8.7 Hz, | C₂₁H₁₈ClF₂O₄ | 407.08498 |
| | 2H), 7.14-7.05 (m, 1H), 7.02 (d, J = 8.6 Hz, | 407.08562 | |
| | 2H), 6.91-6.79 (m, 2H), 6.12-6.05 (m, | | |
| | 1H), 5.61-5.56 (m, 1H), 4.49-4.44 (m, | | |
| | 2H), 4.40-4.32 (m, 2H), 1.93 (t, J = 1.3 Hz, | | |
| | 3H); ¹³C NMR δ 167.14, 166.37, 163.23 (dd, | | |
| | J = 250.1, 11.4 Hz), 160.46 (dd, J = 250.7, | | |
| | 12.5 Hz), 142.06, 136.05, 135.83, 132.66, | | |
| | 132.41 (dd, J = 9.6, 4.7 Hz), 131.44, 128.95, | | |
| | 126.17, 125.90, 119.31 (dd, J = 16.7, 4.2 | | |
| | Hz), 111.96 (dd, J = 21.3, 3.6 Hz), 104.65 | | |
| | (t, J = 25.6 Hz), 63.16, 62.27, 18.36. | | |
| JN152 | ¹H NMR δ 8.01 (br s, 1H), 7.87 (s, 1H), 7.78 | C₂₀H₁₆ClF₃NO₂ | 394.08053 |
| | (d, J = 8.0 Hz, 2H), 7.47 (d, J = 7.9 Hz, 2H), | 394.08162 | |
| | 7.18 (d, J = 8.6 Hz, 2H), 6.94 (d, J = 8.6 Hz, | | |
| | 2H), 5.41 (q, J = 1.6 Hz, 1H), 5.34 (d, J = 1.1 | | |
| | Hz, 1H), 1.83 (t, J = 1.3 Hz, 3H); ¹³C NMR δ | | |
| | 165.83, 164.03, 140.16, 140.07, 138.89, | | |
| | 136.05, 133.23, 132.23, 131.86, 131.73 (q, J = | | |
| | 33.0 Hz), 130.52, 129.04, 127.14 (q, J = | | |
| | 3.7 Hz), 123.74 (q, J = 272.4 Hz), 122.06, | | |
| | 18.21. | | |
| JN153 | ¹H NMR δ 8.73 (d, J = 2.2 Hz, 1H), 8.54 (br | C₁₉H₁₆F₃N₂O₂ | 361.11492 |
| | s, 1H), 7.89 (dd, J = 8.2, 2.1 Hz, 1H), 7.65 | 361.11584 | |
| | (dd, J = 8.2, 0.8 Hz, 1H), 7.52 (dd, J = 8.1, | | |
| | 1.6 Hz, 2H), 7.46-7.36 (m, 3H), 6.94 (s, | | |
| | 1H), 5.61 (q, J = 0.9 Hz, 1H), 5.54 (q, J = 1.6 | | |
| | Hz, 1H), 1.84 (dd, J = 1.6, 0.9 Hz, 3H); | | |
| | ¹³C NMR δ 169.91, 164.97, 149.59,147.21 | | |
| | (q, J = 34.9 Hz), 142.16, 138.91, 136.53, | | |
| | 135.48, 134.60, 129.42, 129.10, 126.76, 124.23, | | |
| | 123.67, 121.57 (q, J = 273.7 Hz), 120.40 | | |
| | (q, J = 2.7 Hz), 18.20. | | |
| JN154 | ¹H NMR δ 7.97 (s, 1H), 7.95 (br s, 1H), 7.77 | C₁₉H₁₂ClF₅NO₂ | 416.04572 |
| | (dq, J = 15.6, 2.0 Hz, 1H), 7.24-7.16 (m, | 416.04712 | |
| | 3H), 7.06-6.99 (m, 2H), 6.97 (d, J = 8.5 Hz, | | |
| | 2H), 6.83 (dq, J = 15.6, 6.6 Hz, 1H); ¹³C NMR δ | | |
| | 164.94, 164.65, 164.24 (dd, J = 254.3, | | |
| | 11.5 Hz), 160.49 (dd, J = 251.9, 12.0 | | |
| | Hz), 143.18, 136.70, 132.84 (dd, J = 9.7, | | |
| | 3.8 Hz), 131.85 (q, J = 35.6 Hz), 131.71, 131.58, | | |
| | 129.91 (q, J = 6.3 Hz), 129.23, 126.48, | | |
| | 122.26 (q, J = 270.2 Hz), 117.45 (dd, J = | | |
| | 16.7, 4.2 Hz), 113.69 (dd, J = 21.5, 3.8 Hz), | | |
| | 106.07 (t, J = 25.3 Hz). | | |
| JN155 | ¹H NMR δ 7.97 (s, 1H), 7.90-7.78 (m, 3H), | C₂₄H₁₇ClF₂NO₂ | 424.08991 |
| | 7.65-7.61 (m, 2H), 7.44-7.38 (m, 3H), | 424.09104 | |
| | 7.25-7.19 (m, 3H), 7.06-6.96 (m, 4H); | | |
| | ¹³C NMR δ 167.34, 164.79, 164.10 (dd, J = | | |
| | 253.5, 11.5 Hz), 160.50 (dd, J = 251.8, 11.9 | | |
| | Hz), 147.02, 141.99, 136.26, 134.70, 132.87 | | |
| | (dd, J = 9.6, 3.9 Hz), 132.43, 131.44, 130.92, | | |
| | 129.13, 129.05, 128.80, 127.58, 119.25, | | |
| | 117.92 (dd, J = 16.6, 4.1 Hz), 113.49 (dd, J = | | |
| | 21.4, 3.8 Hz), 105.94 (t, J = 25.3 Hz). | | |
| JN156 | ¹H NMR (DMSO-d₆) δ 7.90 (br t, J = 5.8 Hz, | C₂₂H₂₂ClF₂N₂O₂ | 419.13168 |
| | 1H), 7.75 (br t, J = 5.9 Hz, 1H), 7.57 (s, 1H), | 419.13324 | |
| | 7.35-7.29 (m, 3H), 7.23 (td, J = 8.5, 6.6 Hz, | | |
| | 1H), 7.13 (td, J = 8.5, 2.6 Hz, 1H), 7.04 (d, J = | | |
| | 8.7 Hz, 2H), 5.65-5.60 (m, 1H), 5.31 (p, J = | | |
| | 1.6 Hz, 1H), 3.15 (q, J = 6.8 Hz, 2H), 3.10 | | |
| | (q, J = 6.8 Hz, 2H), 1.84 (t, J = 1.2 Hz, 3H), | | |
| | 1.60 (p, J = 6.9 Hz, 2H); ¹³C NMR (DMSO-d₆) δ | | |
| | 167.43, 166.07, 162.53 (dd, J = 248.6, | | |
| | 13.6 Hz), 159.77 (dd, J = 247.7, 13.0 Hz), | | |
| | 140.00, 135.26, 133.68, 133.24, 133.05 (dd, | | |
| | J = 9.7, 4.6 Hz), 130.86, 130.30, 128.55, | | |
| | 119.69 (dd, J = 16.6, 4.0 Hz), 118.85, 112.37 | | |
| | (dd, J = 21.6, 3.4 Hz), 104.78 (t, J = 26.0 | | |
| | Hz), 37.07, 36.40, 29.21, 18.62. | | |
| JN053 | ¹H NMR δ 7.40-7.32 (m, 5H), 7.30-7.27 | C₁₉H₁₇O₂ | 277.12072 |
| | (m, 2H), 6.80 (d, J = 8.9 Hz, 2H), 6.24 (q, J = | 277.12231 | |
| | 1.8 Hz, 1H), 5.55 (q, J = 1.4 Hz, 1H), 3.81 | | |
| | (s, 3H), 3.66 (t, J = 1.7 Hz, 2H); ¹³C NMR δ | | |
| | 194.34, 161.55, 161.29, 141.77, 140.23, | | |
| | 133.26, 130.35, 129.59, 128.72, 127.99, | | |
| | 127.40, 116.59, 114.02, 55.47, 35.11. | | |

1. Unless otherwise specified, the NMR data are given in chloroform-d at 500 MHz for ¹H NMR, and at 126 MHz for ¹³C NMR. 2. Unless otherwise specified, formula is for [M + H]⁺ where M represents the compound in its charge neutral form.

### Example 1: XRPD Crystals and Characterization

X-ray quality crystals of selected compounds were grown according to the following general method: The compound (˜2-10 mg) was placed in a vial, dissolved in a minimal amount (0.25-0.50 mL) of dichloromethane, and then diluted with hexanes (0.50-1.0 mL). The resultant solution was allowed to concentrate via slow evaporation to result in the growth of x-ray quality crystals, which were left in the mostly-hexanes containing mother liquor until further analysis.

XRPD spectra for compounds JN032, JN110, JN034, JN097, JN117, and JN103 are shown in FIGS. 4-9, respectively. Acquisition parameters are described in Appendix A.

### Example 2: Biological Assays Conducted on Exemplary Compounds

JN053 and JN138-JN156 were synthesized and tested in biochemical and cell biologic assays that have been described elsewhere. [1-6] The JN compounds were first studied in cell viability assays (MTT assay) to determine the efficacy and specificity of the JN compounds for inhibition of prostate cancer cell lines.

The growth inhibitory effect of the JN compounds was assessed through the MTT assay, which assesses the total number of viable cells in vitro. These experiments were performed in AR-expressing (AR-positive) prostate cancer cell lines to assess on target effects and AR-null (AR-negative) prostate cancer cell lines to assess off target effects (i.e. specificity).

AR-expressing (AR-pos):
LNCaP: full length AR
LNCaP AR: overexpression of full length AR
22Rv1: full length AR and ARV7
VCaP: full length AR and ARV7
CWR22: full length AR and ARV7

### AR-Negative:

PC3
DU145

Only those compounds that exhibit strong inhibition of AR-expressing (AR-positive) prostate cancer cell lines and minimal inhibition of AR-null (AR-negative) prostate cancer cell lines were subjected to biochemical assays to assess inhibitory effects on AR transcriptional activity.

The biochemical assays include reporter assays to determine the activity and specificity of these JN compounds to inhibit the transcriptional activity of the androgen receptor (AR). The reporter assays were conducted in various cell lines that either endogenously or exogenously express the full-length AR and ARV7, a constitutively active splice variant that is resistant to all clinically available AR targeting compounds. The reporter assays were performed in replicates and across a wide range of concentrations (generally 0-10 mM).

The reporter systems utilized include:
MMTV-luciferase: AR-dependent
ARE-luciferase: AR-dependent
GRE-luciferase: glucocorticoid receptor (GR)-dependent
CRE-luciferase: CREB-dependent
AP1-luciferase: AP1 (jun and fos family)-dependent:
AR-TAD-luciferase: dependent on AR transactivation domain
CREB-TAD-luciferase: dependent on CREB transactivation domain
JUN-TAD-luciferase: dependent on c-Jun transactivation domain

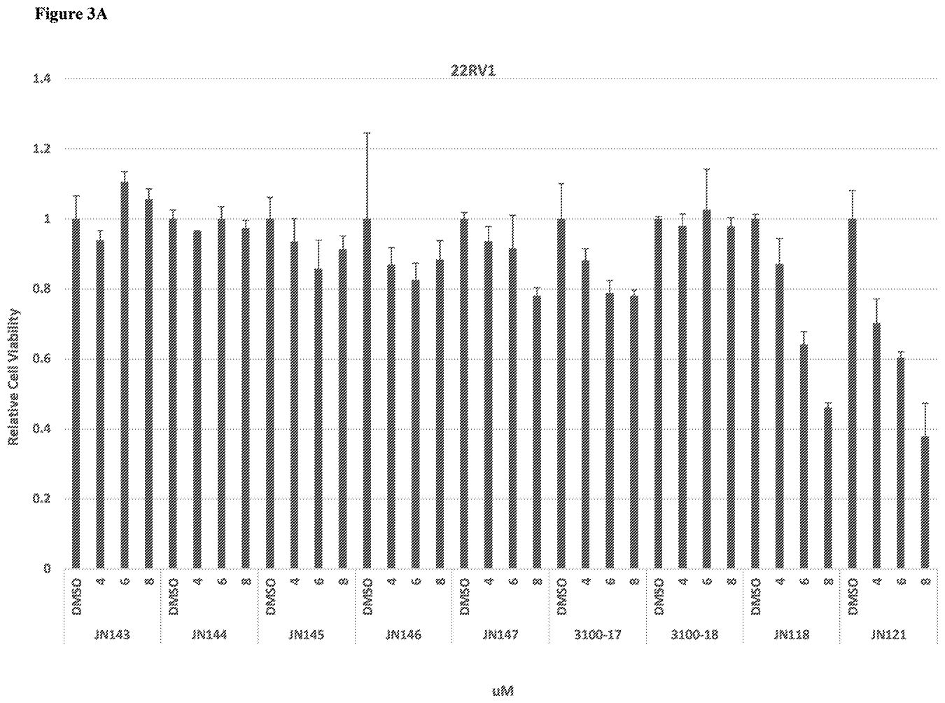

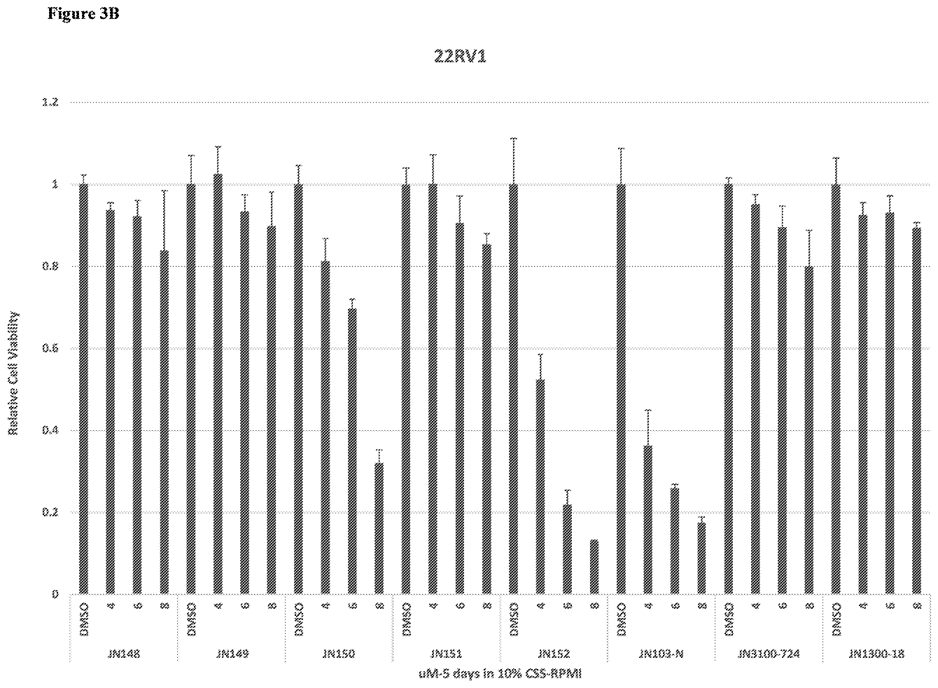

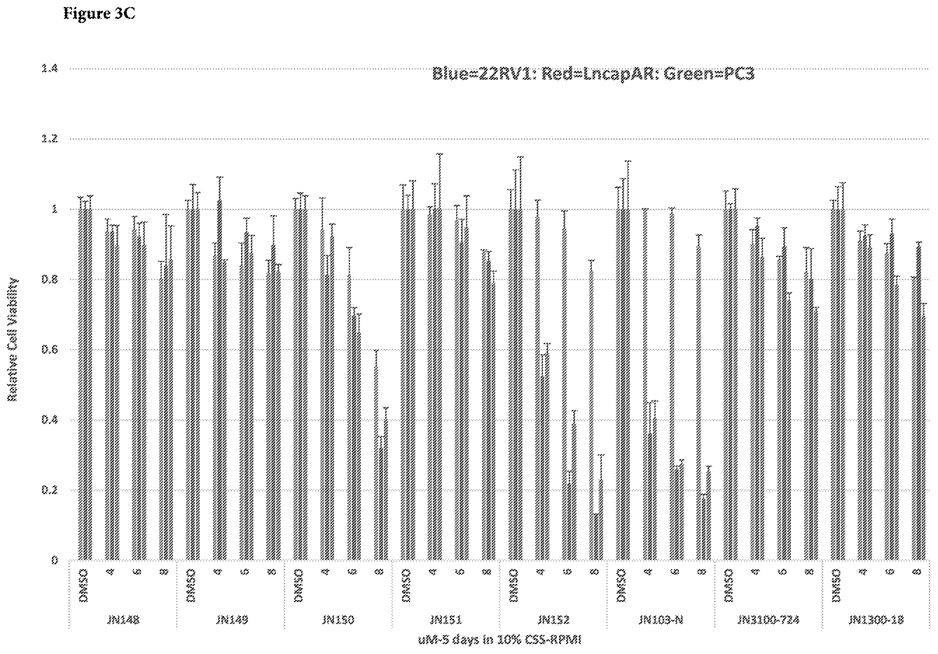

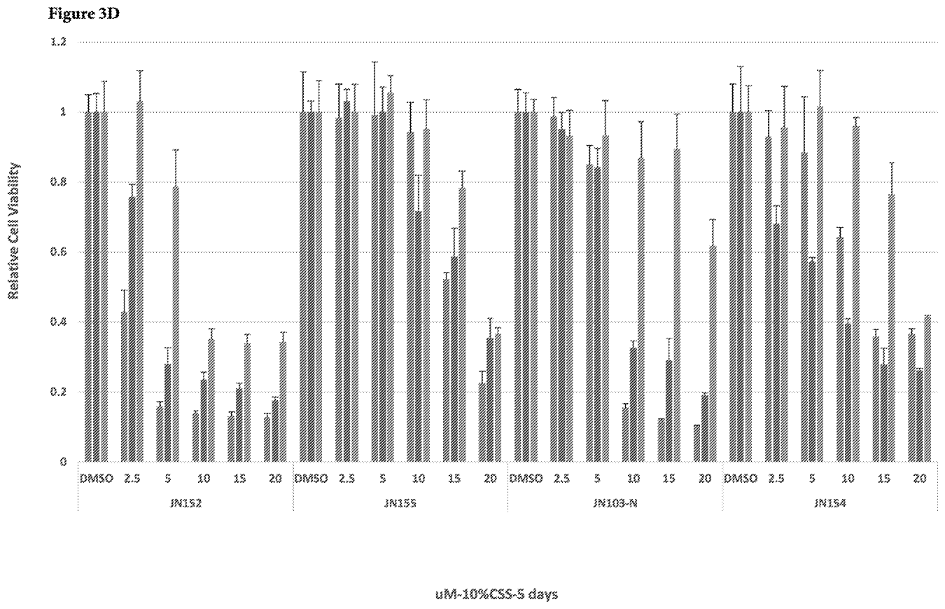

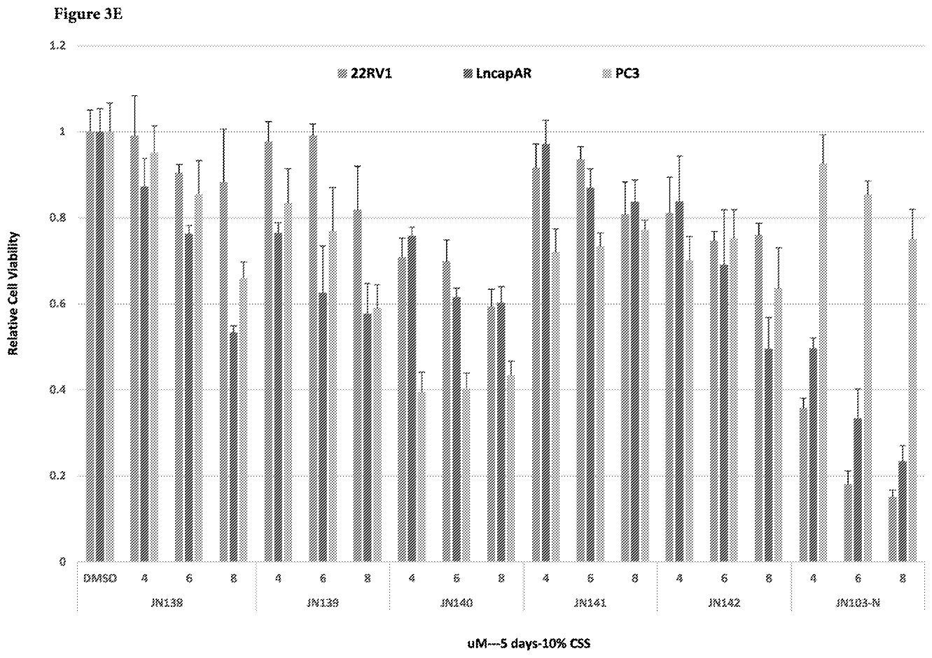

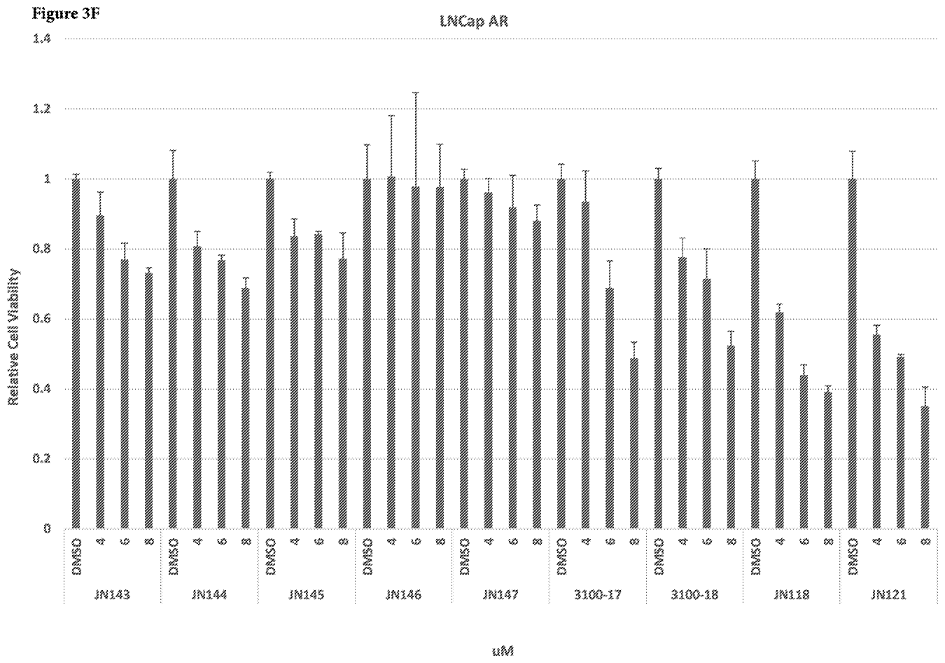

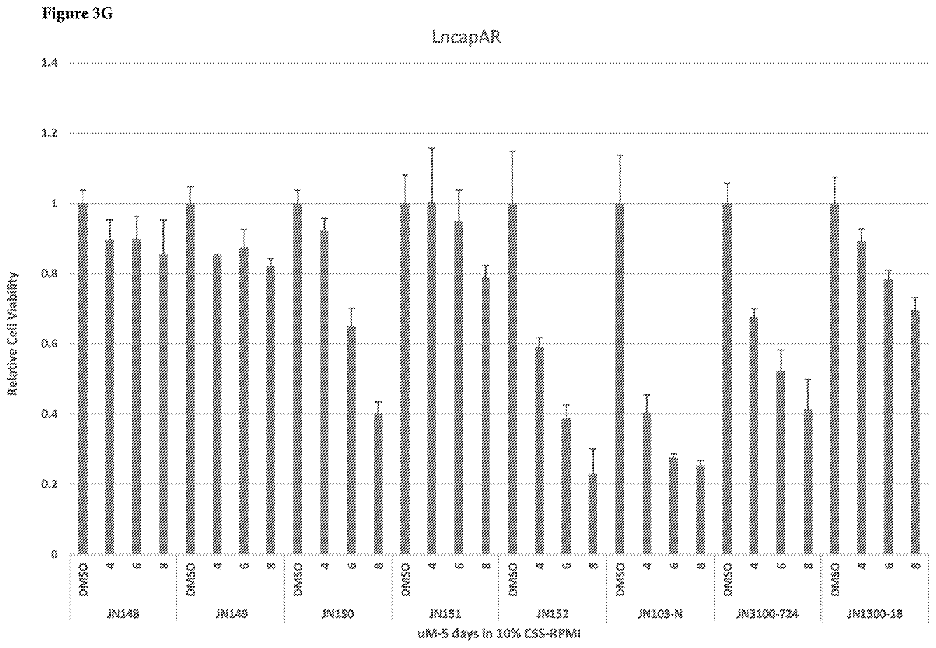

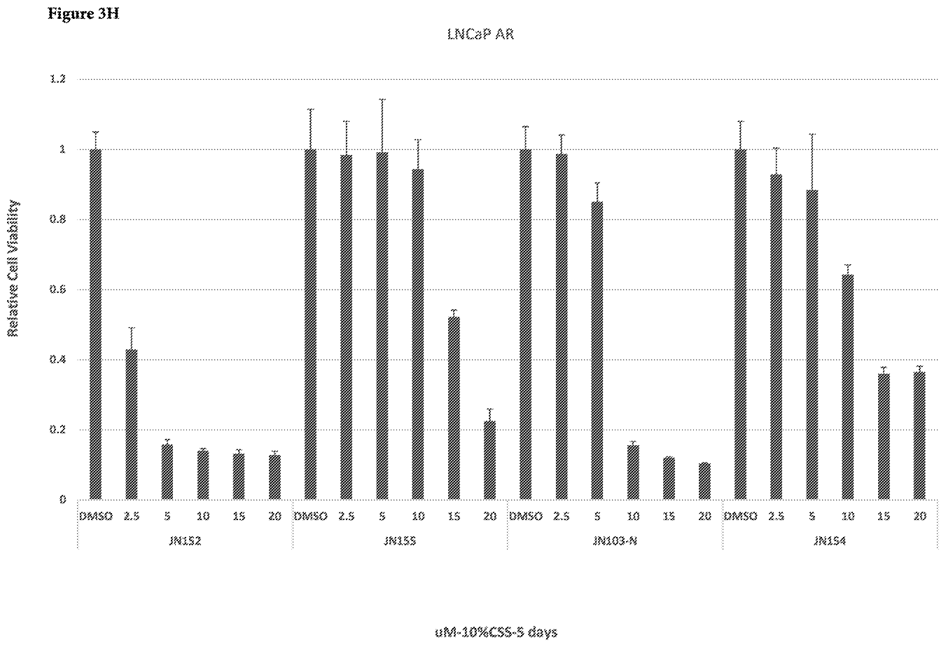

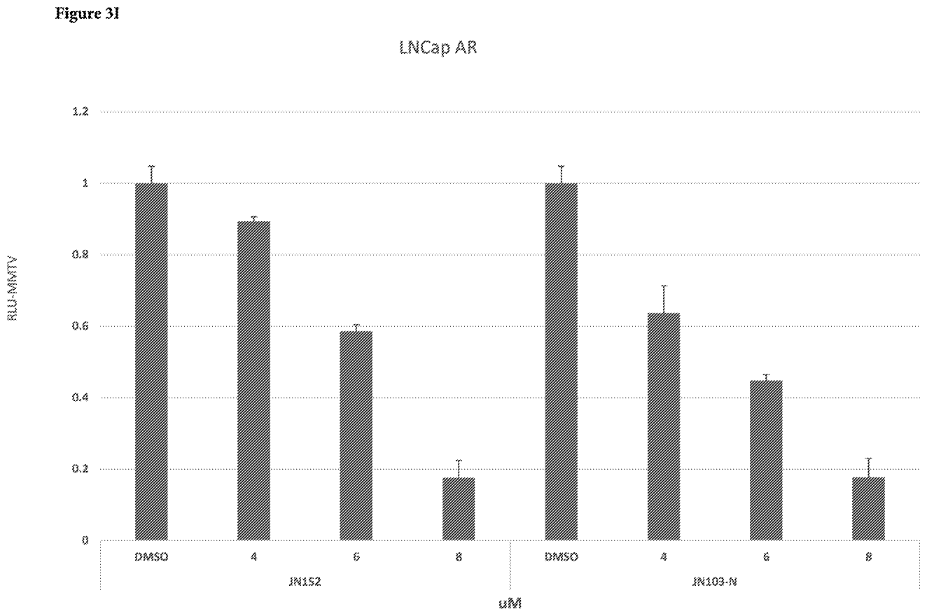

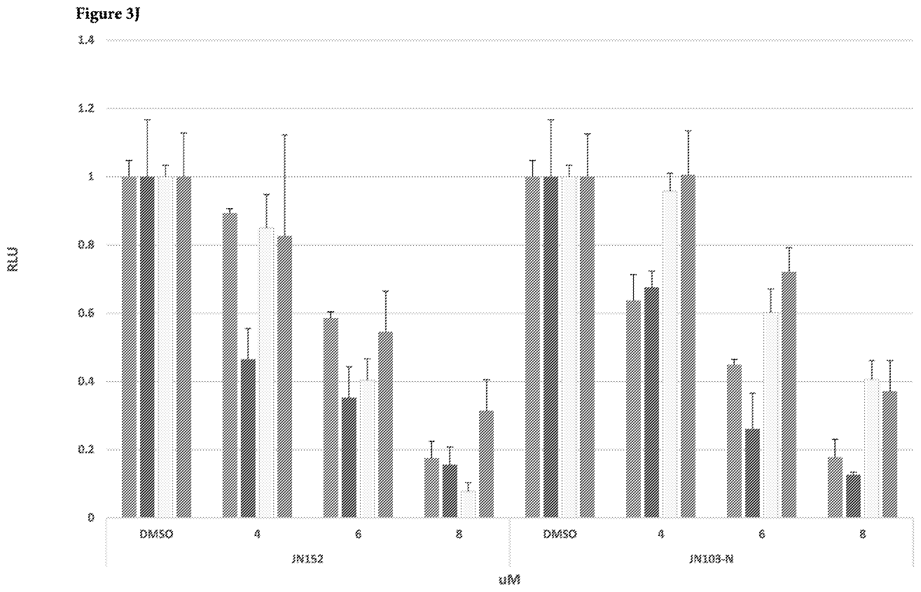

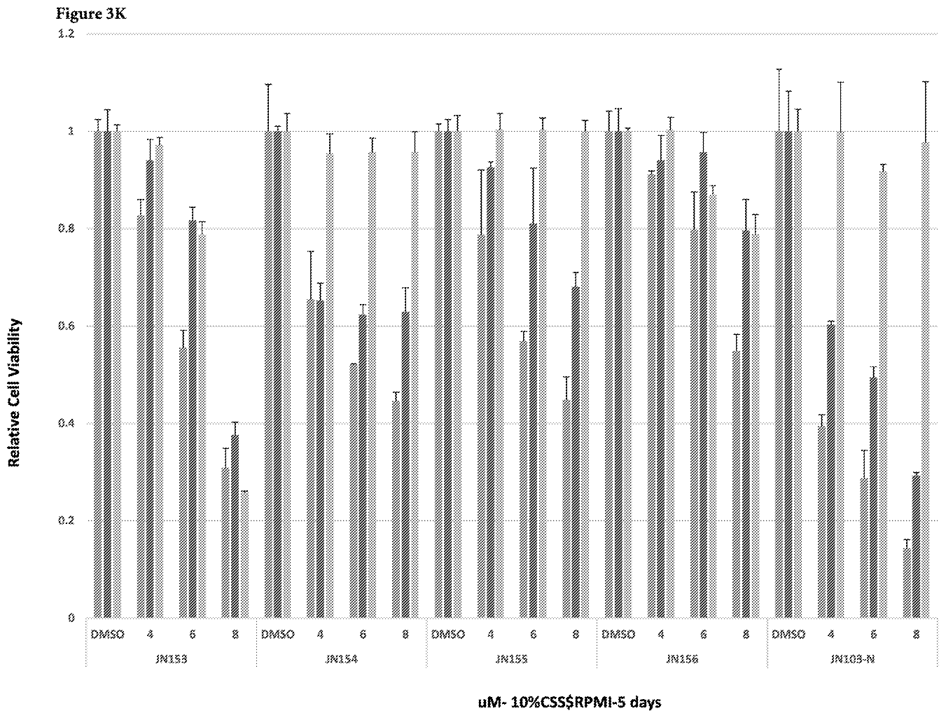

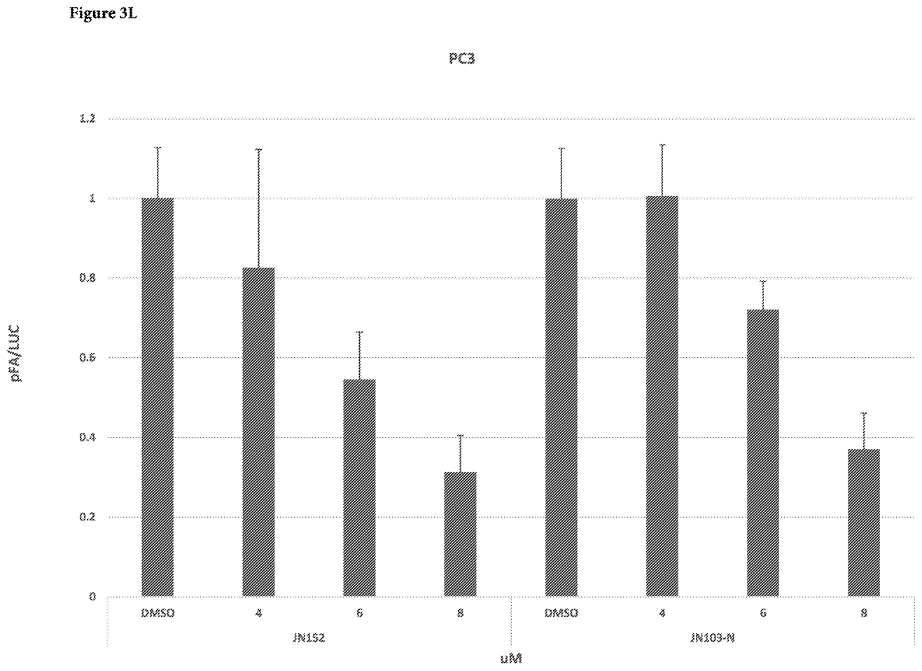

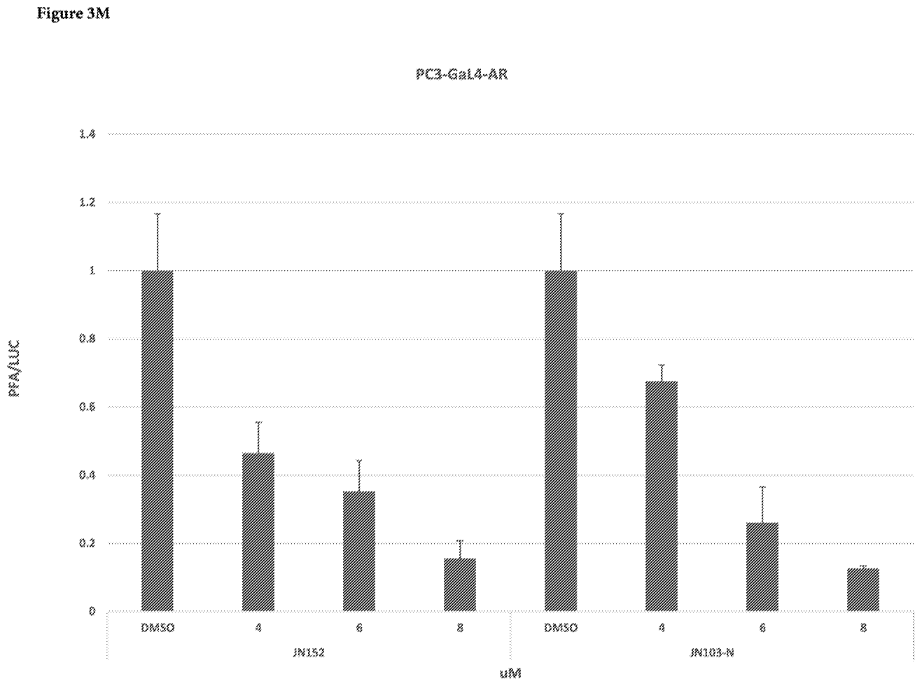

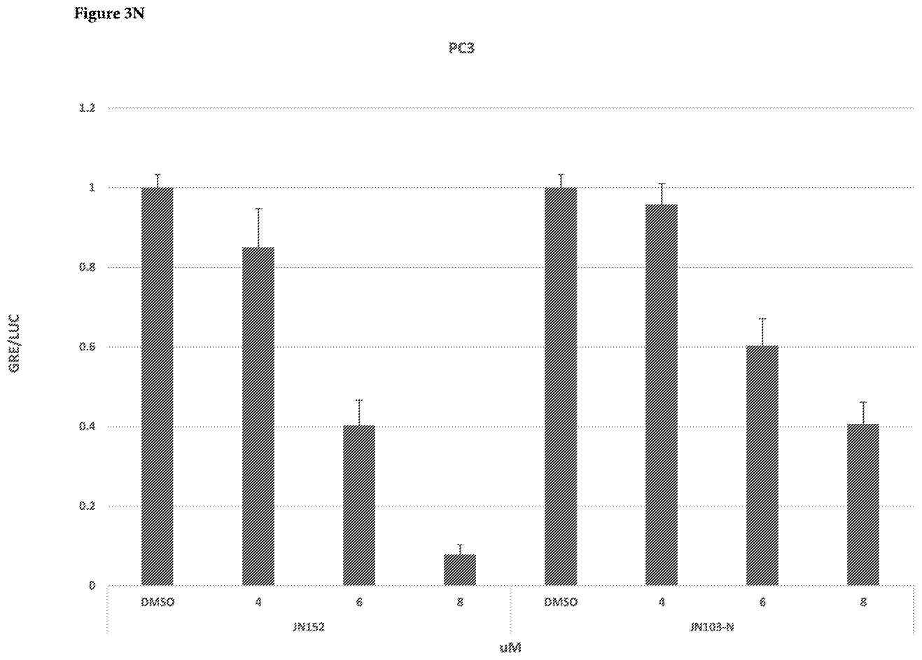

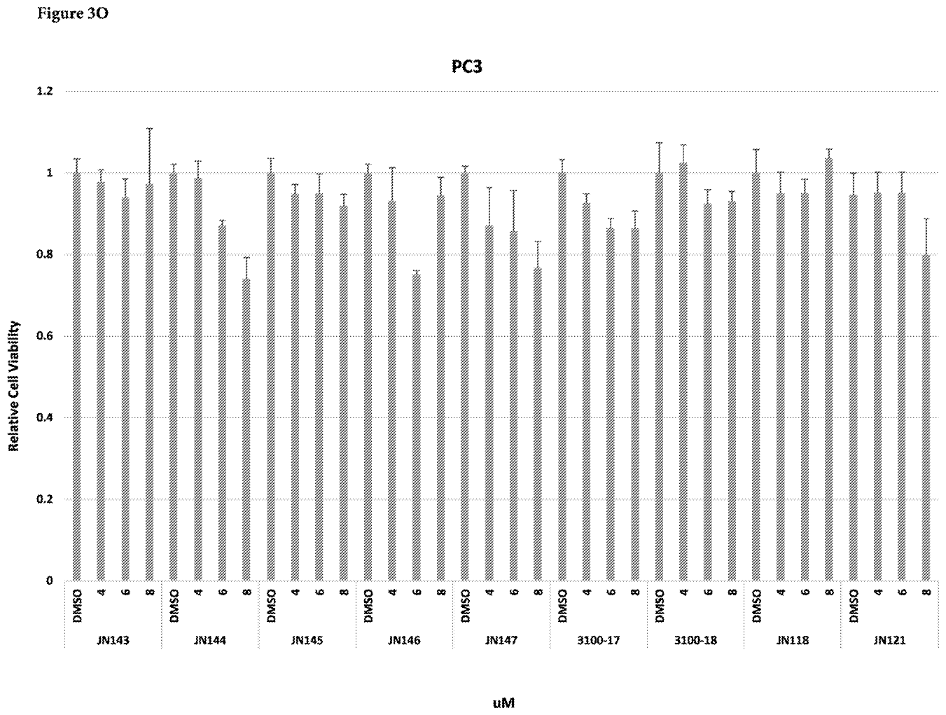

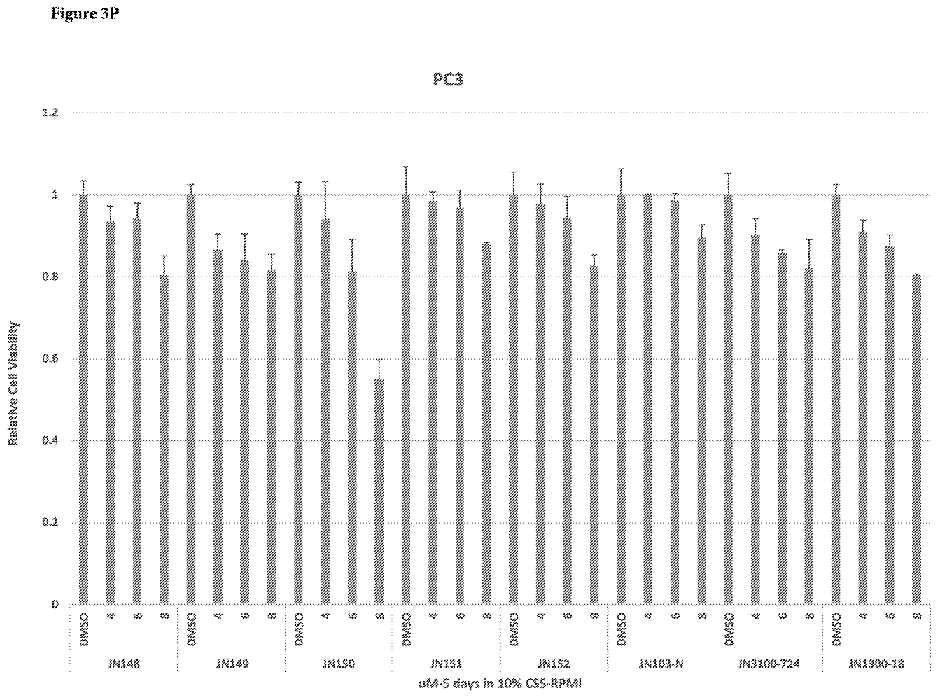

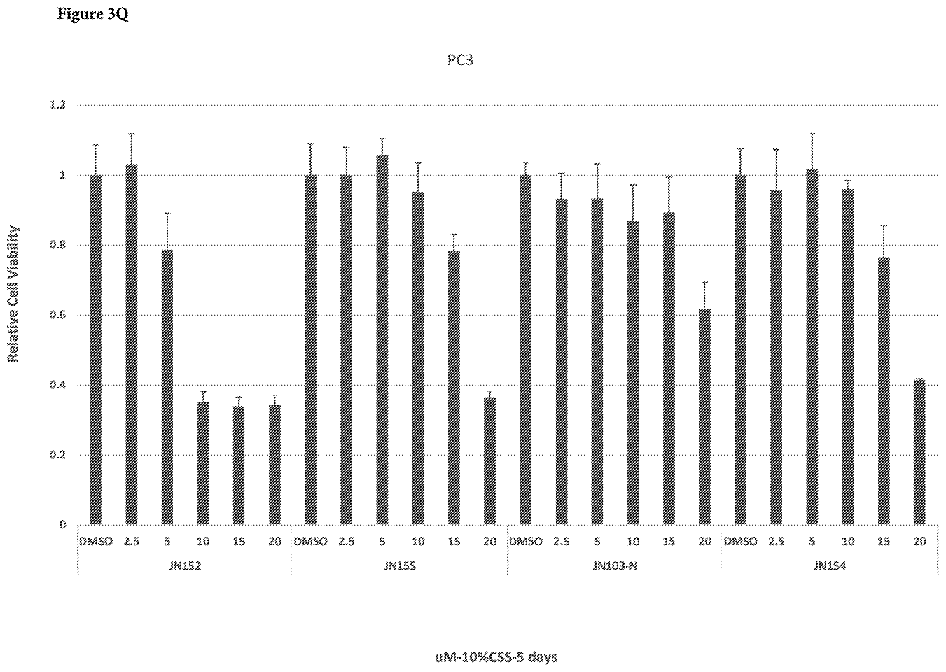

The data for the reporter assays and MTT assays are summarized in the table below. (FIGS. 3A-Q).

| | | | | AR- | JUN- | CREB- | | | MTT AR- | MTT AR |
|---|---|---|---|---|---|---|---|---|---|---|
| | MMTV | ARE | GRE | TAD | TAD | TAD | CRE | AP1 | Pos | Neg |
| MDV3100 | HIGH | HIGH | LOW | LOW | LOW | LOW | | HIGH | HIGH | LOW |
| JN138 | | | | | | | | | LOW | LOW |
| JN139 | | | | | | | | | LOW | LOW |
| JN140 | | | | | | | | | HIGH | HIGH |
| JN141 | | | | | | | | | LOW | LOW |
| JN142 | | | | | | | | | LOW | LOW |
| JN143 | | | | | | | | | LOW | LOW |
| JN144 | | | | | | | | | LOW | LOW |
| JN145 | | | | | | | | | LOW | LOW |
| JN146 | | | | | | | | | LOW | LOW |
| JN147 | | | | | | | | | LOW | LOW |
| JN148 | | | | | | | | | LOW | LOW |
| JN149 | | | | | | | | | LOW | LOW |
| JN150 | | | | | | | | | HIGH | HIGH |
| JN151 | | | | | | | | | LOW | LOW |
| JN152 | HIGH | | HIGH | HIGH | | HIGH | | | HIGH | LOW |
| JN153 | | | | | | | | | HIGH | HIGH |
| JN154 | | | | | | | | | HIGH | LOW |
| JN155 | | | | | | | | | LOW | LOW |
| JN156 | | | | | | | | | LOW | LOW |
| JN053 | MED | | | | | | | | LOW | LOW |

### Example 3: JN103 Inhibits AR Transcriptional Readout

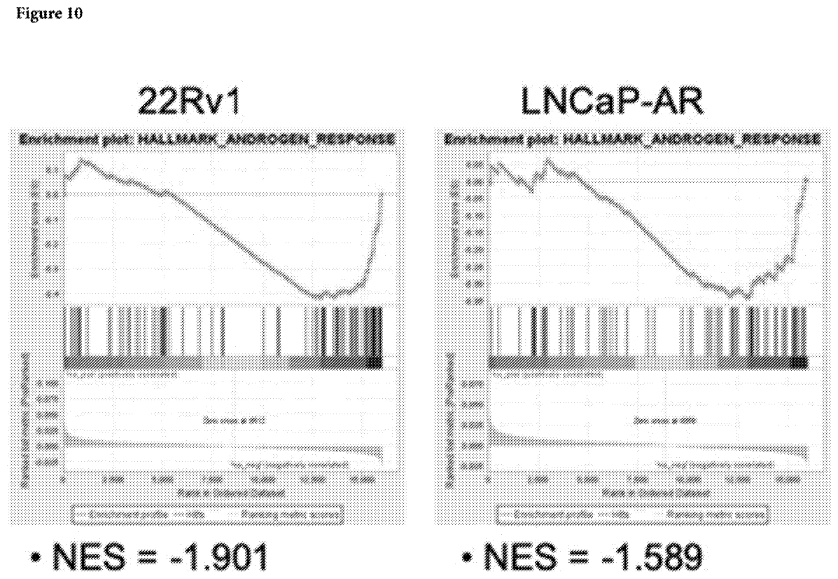

Whole transcriptomic RNA-sequencing was performed for two castration resistant cell lines (LNCaP-AR and 22Rv1), which were exposed to JN103 (10 μM) for 8 hours. The experimental results as determined by gene set expression analysis (FIG. 10) show negative enrichment scores (NES) for the AR transcriptional program. The results demonstrate a marked decrease in the AR gene signature.

### Example 4: JN103 Selectively Induces Degradation of AR

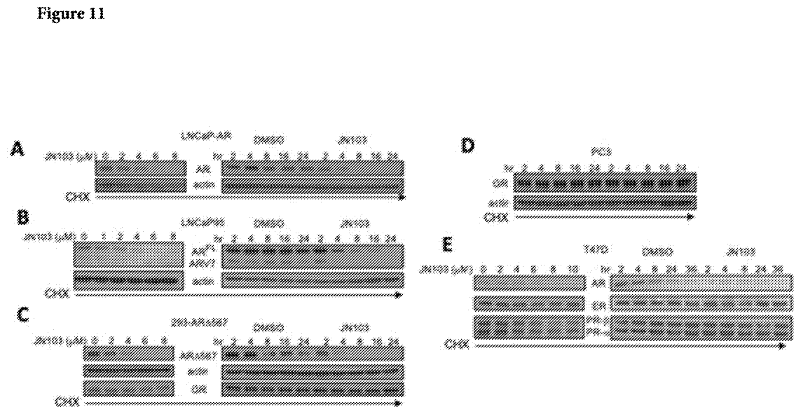

LNCaP-AR cells were treated with JN103 and cycloheximide (to inhibit translation) at the indicated doses and times (FIG. 11A). Cell protein was subjected to Western blotting for the indicated proteins. The test results are shown in FIG. 11A. The same tests were performed on LNCaP-95 cells, HEK-293 cells engineered to ectopically express ARΔ567, PC3 cells, and T47D breast cancer cells. The experimental results obtained from these tests are shown in FIGS. 11B-11E, respectively.

The test results show that JN103 potently induces time- and dose-dependent degradation of: 1) the full-length AR overexpressed in LNCaP-AR cells (FIG. 11A), 2) the full-length AR and AR-V7 (constitutively active AR splice variant) endogenously expressed in LNCaP-95 cells, and 3) ARΔ567 (also a constitutively active AR variant) ectopically expressed in HEK-293 cells. Importantly, JN103 does not affect the degradation of other proteins, including actin or the GR (FIGS. 11(A)-11(D)). In addition, JN103 induces degradation of the AR but not the ER or PR in a breast cancer cell line that co-expresses AR, ER, and PR (FIG. 11(E)).

### Example 5: Selective Growth Inhibitory Effects of JN103 on AR-Expressing Cancer Cells

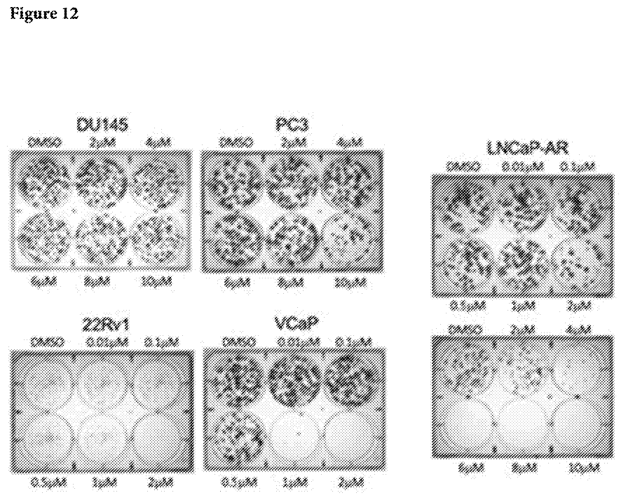

DU145, PC3LNCaP-AR (full-length AR), 22Rv1 (full-length and splice variant AR), and VCaP cells (400 cells/well of 6-well plate) were treated with indicated 0 (pure DMSO), 2, 4, 6, 8, 10 μm of JN103 for two weeks. Colonies were visualized with methylene blue staining. Colony formation assays show that JN103 inhibits the growth of castration resistant AR expressing cells, including LNCaP-AR (full-length AR), 22Rv1 (full-length and splice variant AR), and VCaP (full-length and splice variant AR) (FIG. 12). However, JN103 has limited impact on colony formation of castration resistant, AR-null DU145 cells and only mild effects on PC3 cell colony formation at high concentrations (FIG. 12).

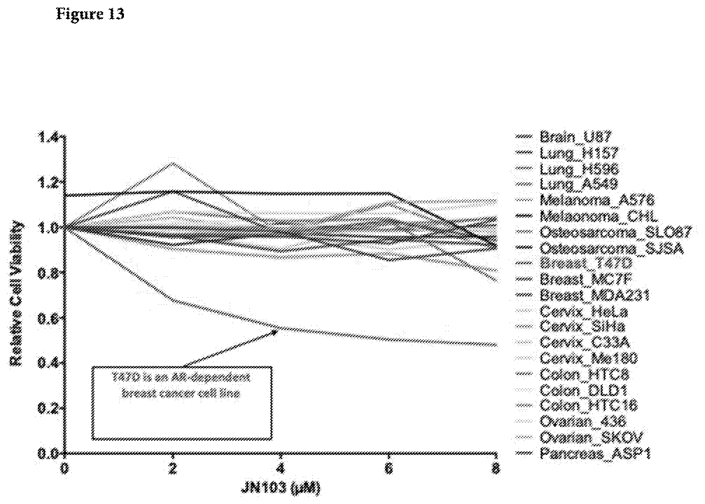

The growth-inhibitory effects of JN103 were also assessed in MTT assays on 20 non-prostate cancer cell lines. (FIG. 13). Cells were exposed to 0 (pure DMSO), 2, 4, 6, and 8 μm of JN103 for 5 days and subjected to MTT assay to determine cell viability. Results are means of quadruplicates. According to FIG. 13, JN103 exhibits significant growth inhibition of a breast cancer cell line (T47D), which expresses the full-length AR and is dependent upon AR expression for growth.

### REFERENCES

1. An J, Fisher M, Rettig M B. VHL Expression in renal cell carcinoma markedly sensitizes to bortezomib (PS-341) through a NF-κB-dependent mechanism. Oncogene. 24:1563-70, 2005.
2. An J and Rettig M B. Mechanism of von Hippel-Lindau Protein-Mediated Suppression of Nuclear Factor kappa B (NF-κB) Activity. Mol Cell Biol. 25:7546-56, 2005.
3. An J, Mo N, Rettig M B. EGFR inhibition sensitizes renal cell carcinoma cells to bortezomib. Mol Can Ther. 6:61-9, 2007.
4. Rettig, M B, Heber D, An J, Seeram N P, Rao J Y, Liu H, Klatte T, Belldegrun A, Moro A, Henning S M, Mo D, Aronson, W J, Pantuck, A. Pomegranate Extract Inhibits NF-κB Activation and Delays the Emergence of Androgen-Independence in the LAPC4 Prostate Cancer Xenograft Model. Molecular Cancer Therapeutics. 7:2662, 2008.
5. Pantuck A J, An J, Liu H, Rettig M B. NF-kappa B Dependent Plasticity of the Epithelial to Mesenchymal Transition Induced by VHL Inactivation in Renal Cell Carcinomas. Cancer Research, 70:752, 2010.
6. An J, Liu H, Magyar C E, Guo Y, Veena M S, Srivatsan E S, Huang J, Rettig M B. Hyperactivated c-Jun N-terminal Kinase as a Therapeutic Target in pVHL-Deficient Renal Cell Carcinomas. Cancer Research 2013 Feb. 15; 73(4):1374-85.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

Those skilled in the art will recognize, or be able to ascertain using no more than routine experimentation, numerous equivalents to the compounds and methods of use thereof described herein. Such equivalents are considered to be within the scope of this invention and are covered by the following claims. Those skilled in the art will also recognize that all combinations of embodiments described herein are within the scope of the invention.

We claim:

1. A compound having the structure of formula I, II, III, IV, V, VI, VII, or VIII, or a pharmaceutically acceptable salt thereof: 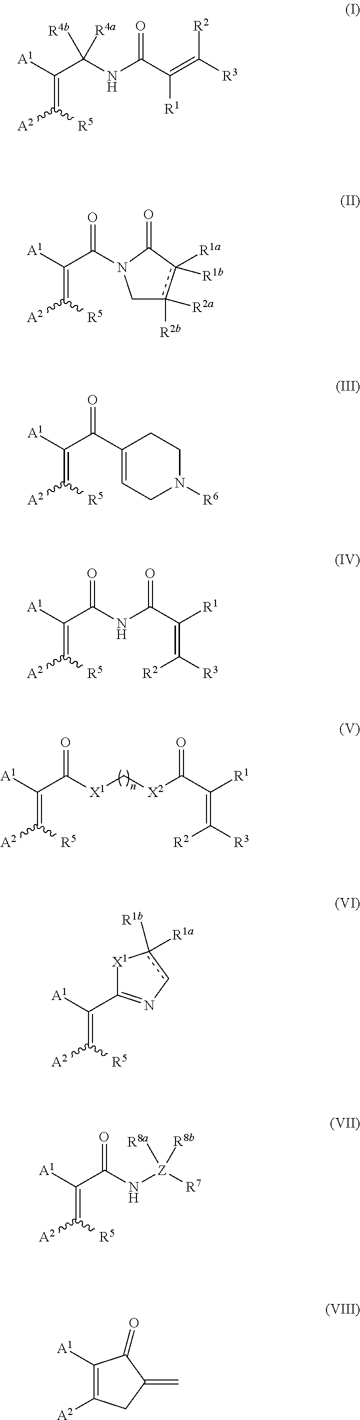
wherein:
A¹ is aryl or hetaryl;
A² is aryl or hetaryl;
R⁵ is H, alkyl, or halo;
R¹ is H, alkyl, haloalkyl, aralkyl, or hetaralkyl;
R² is H, alkyl, or haloalkyl;
R³ is H, alkyl, haloalkyl, aryl, or hetaryl;
R^{4a} and R^{4b} are each independently H or alkyl, or R^{4a} and R^{4b} combine to form oxo; [symbol]
is a single bond or a double bond,
when [symbol] is a single bond in Formula (II), R^{1a}, R^{1b}, R^{2a}, and R^{2b} are each independently H, alkyl, or alkoxy;
when [symbol] is a double bond in Formula (II),
R^{1a} and R^{2a} are each independently H, alkyl, or alkoxy, and
R^{1b} and R^{2b} are absent;
when [symbol] is a single bond in Formula (VI), R^{1a} and R^{1b} combine to form CH₂;
when [symbol] is a double bond in Formula (VI), R^{1a} is H or alkyl and R^{1b} is absent;
R⁶ is H, alkyl, aralkyl, or hetaralkyl;
X¹ and X² are each independently NH or O;
n is 1-4;
X is O, NH, or S;
R⁷ is amino, alkynyl, cyano, cycloalkyl, alkyl, or alkenyl;
Z is S or C;
when Z is S, R^{8a} and R^{8b} are each oxo;
when Z is C,
R^{8a} and R^{8b} are each independently H or alkyl, or
R^{8a} and R^{8b} combine to form oxo, or
R^{8a} and R^{8b} combine to form a cyclopropyl ring including Z.

2. The compound of claim 1, wherein, when A¹ and A² are both phenyl in Formula (VIII), at least one of A¹ and A² is substituted.

3. The compound of claim 1, wherein the compound has the structure of formula (Ia), (Ib), (IIa), (IIb), (IIc), (Va), (Vb), (VIa), (VIb), (VIIa), (VIIb), or (VIIc):

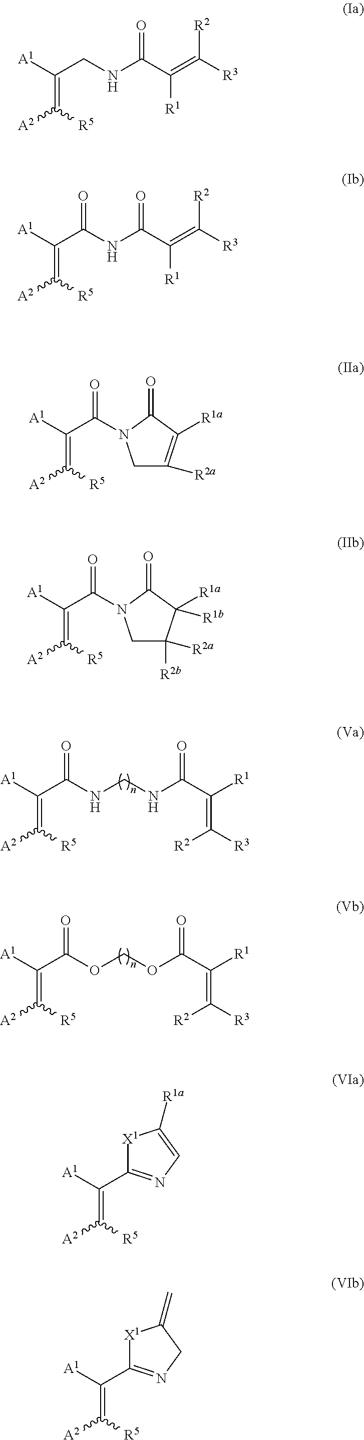

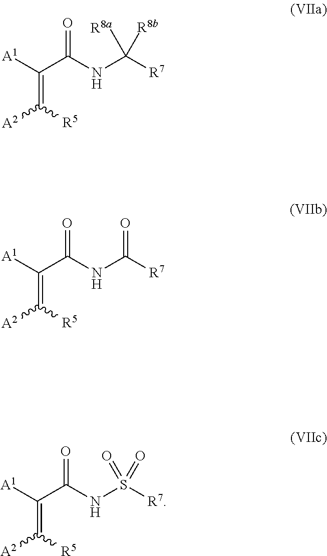

4. The compound of claim 1, wherein the compound is represented by formula I.

5. The compound of claim 1, wherein the compound is represented by formula II.

6. The compound of claim 1, wherein the compound is represented by formula III.

7. The compound of claim 1, wherein the compound is represented by formula IV.

8. The compound of claim 1, wherein the compound is represented by formula V.

9. The compound of claim 1, wherein the compound is represented by formula VI.

10. The compound of claim 1, wherein the compound is represented by formula VII.

11. The compound of claim 1, wherein the compound is represented by formula VIII.

12. The compound of claim 3, wherein the compound is represented by formula Ia.

13. The compound of claim 3, wherein the compound is represented by formula Ib.

14. The compound of claim 3, wherein the compound is represented by formula IIa.

15. The compound of claim 3, wherein the compound is represented by formula IIb.

16. The compound of claim 3, wherein the compound is represented by formula Va.

17. The compound of claim 3, wherein the compound is represented by formula Vb.

18. The compound of claim 3, wherein the compound is represented by formula VIa.

19. The compound of claim 3, wherein the compound is represented by formula VIb.

20. The compound of claim 3, wherein the compound is represented by formula VIIa.

21. The compound of claim 3, wherein the compound is represented by formula VIIb.

22. The compound of claim 3, wherein the compound is represented by formula VIIc.

23. The compound of any one of the preceding claims, wherein A¹ and A² are cis to each other.

24. The compound of any one of the preceding claims, wherein A² is aryl unsubstituted or substituted with one or more R¹¹, wherein each R¹¹ is independently selected from halo, alkyl, haloalkyl, hydroxyl, cyano, alkoxy, alkynyl, or azido.

25. The compound of claim 24, wherein A² is chlorophenyl.

26. The compound of any one of claims 1-23, wherein A² is heteroaryl unsubstituted or substituted with one or more R¹¹, wherein each R¹¹ is independently selected from halo, alkyl, haloalkyl, hydroxyl, cyano, alkoxy, alkynyl, or azido.

27. The compound of claim 26, wherein A² is pyridyl substituted with trifluoromethyl.

28. The compound of any one of the preceding claims, wherein A¹ is phenyl.

29. The compound of any one of the preceding claims, wherein A¹ is unsubstituted.

30. The compound of any of claims 1-28, wherein A¹ is unsubstituted or substituted with at least one R¹² wherein each R¹² is independently selected from halo, alkyl, haloalkyl, hydroxyl, cyano, alkoxy, alkynyl, or azido.

31. The compound of claim 30, herein A¹ is substituted by at least one R¹².

32. The compound of any one of the preceding claims, wherein R⁵ is H or alkyl.

33. The compound of any one of the preceding claims, wherein R⁵ is H.

34. The compound of any one of the preceding claims, wherein R¹ is H or methyl.

35. The compound of any one of the preceding claims, wherein R² is H.

36. The compound of any one of the preceding claims, wherein R³ is H, haloalkyl, or aryl.

37. The compound of any one of the preceding claims, wherein R^{4a} and R^{4b} are each H.

38. The compound of any one of claims 1-36, wherein R^{4a} and R^{4b} combine to form an oxo.

39. The compound of any one of the preceding claims, wherein the compound is of formula III and R⁶ is aryl.

40. The compound of any one of claims 1-38, wherein R⁶ is benzyl.

41. The compound of any one of the preceding claims, wherein the compound is of formula IV and R³ is H, haloalkyl, or aryl.

42. The compound of claim 41, wherein R³ is H, trifluoromethyl, or phenyl.

43. The compound of claim 41 or claim 42, wherein R¹ is H, methyl, or benzyl.

44. The compound of any one of claims 41-43, wherein R¹ and R² are trans to each other.

45. The compound of any one of the preceding claims, wherein the compound is:

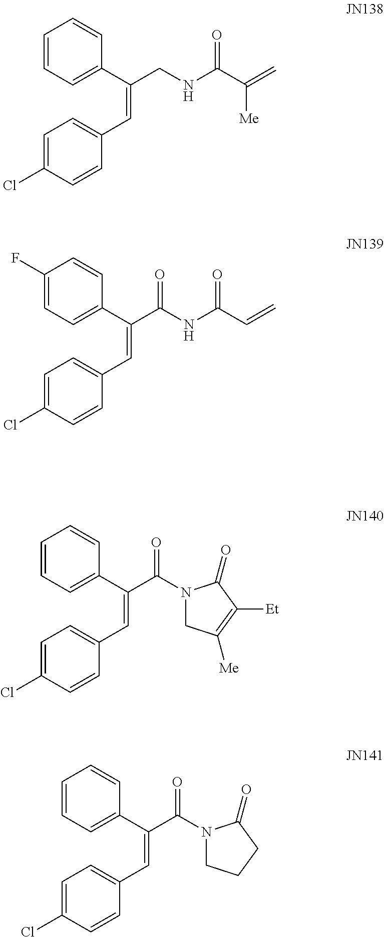 [SMILES: C=C(C)C(=O)NC/C(=C/c1ccc(Cl)cc1)c1ccccc1 ; C=CC(=O)NC(=O)/C(=C/c1ccc(Cl)cc1)c1ccc(F)cc1 ; CCC1=C(C)CN(C(=O)/C(=C/c2ccc(Cl)cc2)c2ccccc2)C1=O ; O=C1CCCN1C(=O)/C(=C/c1ccc(Cl)cc1)c1ccccc1]

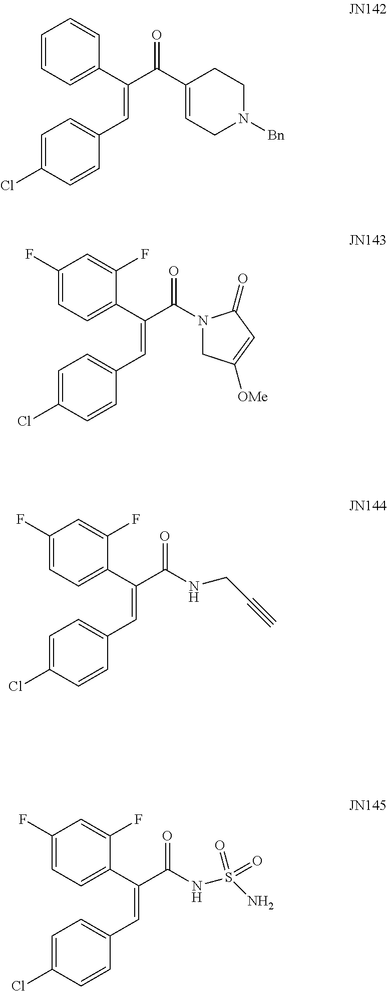 [SMILES: O=C(C1=CCN(Cc2ccccc2)CC1)/C(=C/c1ccc(Cl)cc1)c1ccccc1 ; COC1=CC(=O)N(C(=O)/C(=C/c2ccc(Cl)cc2)c2ccc(F)cc2F)C1 ; C#CCNC(=O)/C(=C/c1ccc(Cl)cc1)c1ccc(F)cc1F ; NS(=O)(=O)NC(=O)/C(=C/c1ccc(Cl)cc1)c1ccc(F)cc1F]

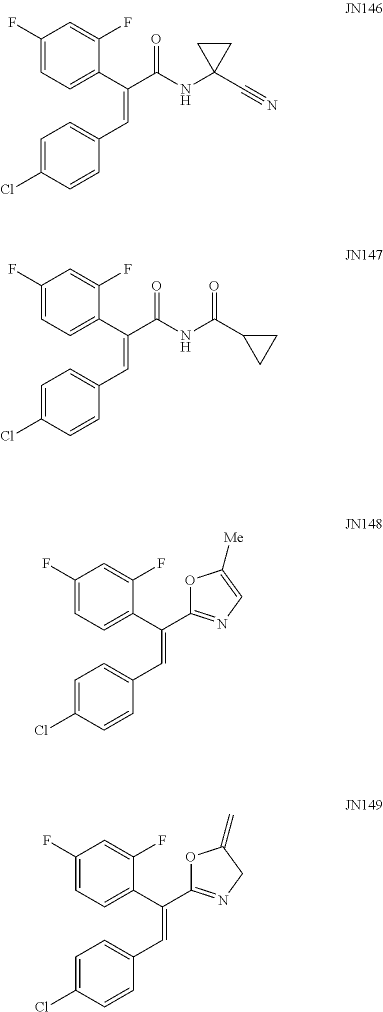 [SMILES: N#CC1(NC(=O)/C(=C/c2ccc(Cl)cc2)c2ccc(F)cc2F)CC1 ; O=C(NC(=O)C1CC1)/C(=C/c1ccc(Cl)cc1)c1ccc(F)cc1F ; Cc1cnc(/C(=C/c2ccc(Cl)cc2)c2ccc(F)cc2F)o1 ; C=C1CN=C(/C(=C/c2ccc(Cl)cc2)c2ccc(F)cc2F)O1]

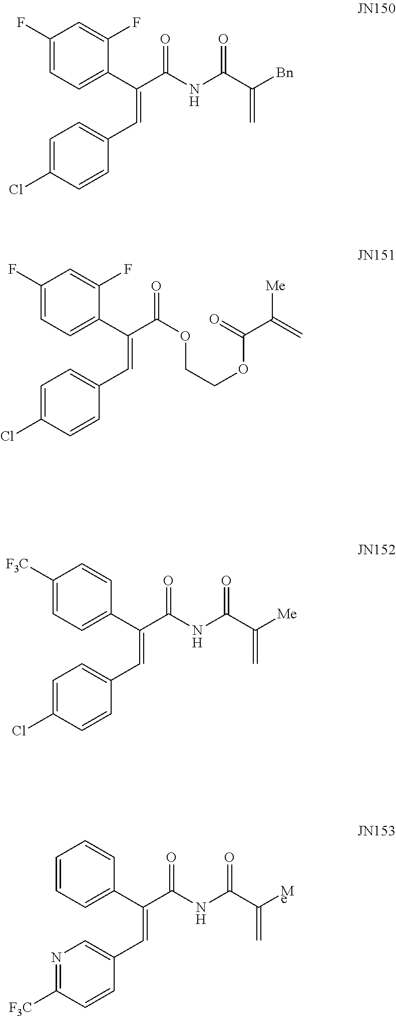 [SMILES: C=C(Cc1ccccc1)C(=O)NC(=O)/C(=C/c1ccc(Cl)cc1)c1ccc(F)cc1F ; C=C(C)C(=O)OCCOC(=O)/C(=C/c1ccc(Cl)cc1)c1ccc(F)cc1F ; C=C(C)C(=O)NC(=O)/C(=C/c1ccc(Cl)cc1)c1ccc(C(F)(F)F)cc1 ; C=C(C)C(=O)NC(=O)/C(=C/c1ccc(C(F)(F)F)nc1)c1ccccc1]

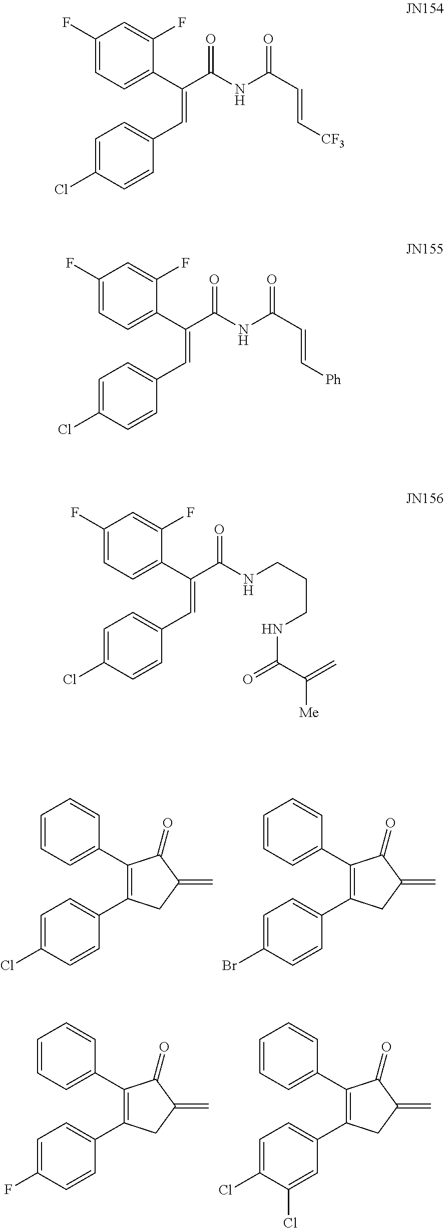

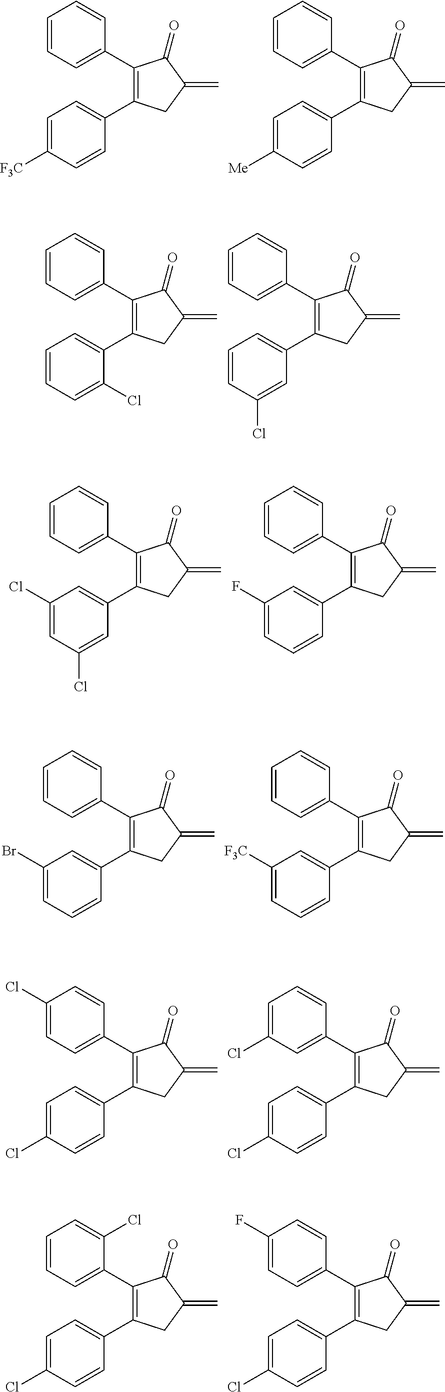

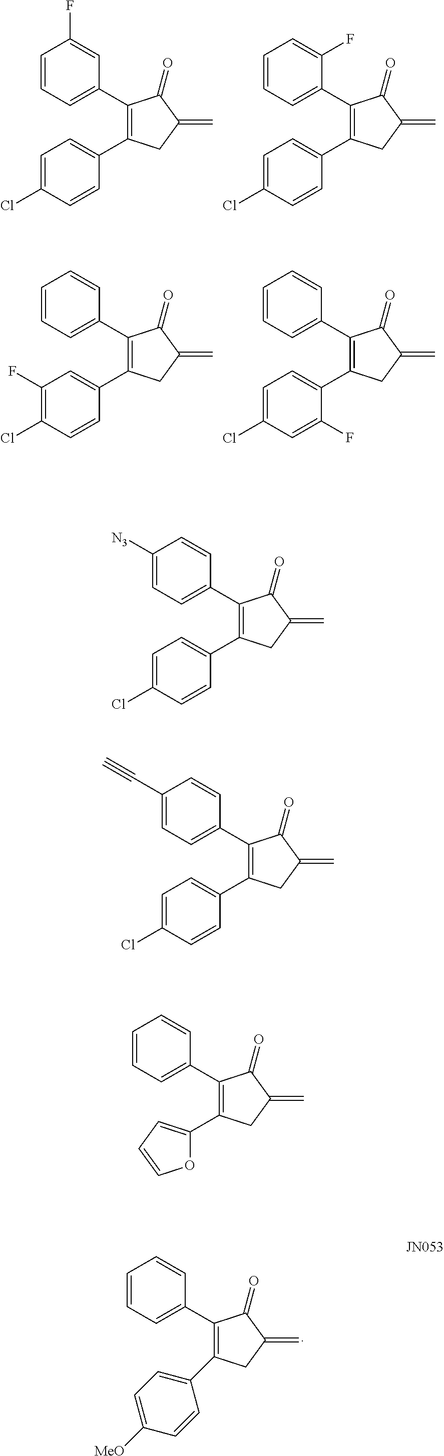

46. The compound of claim 45, wherein the compound is:

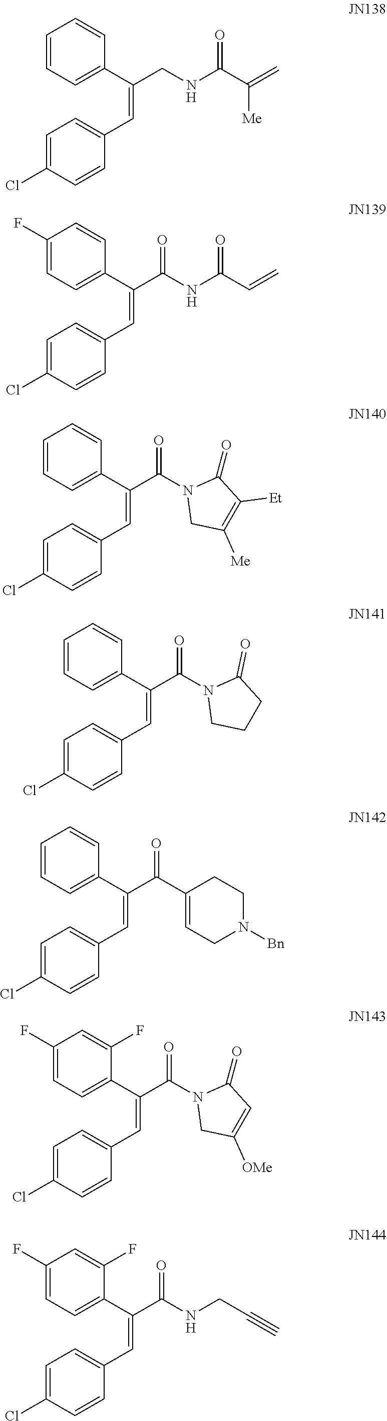

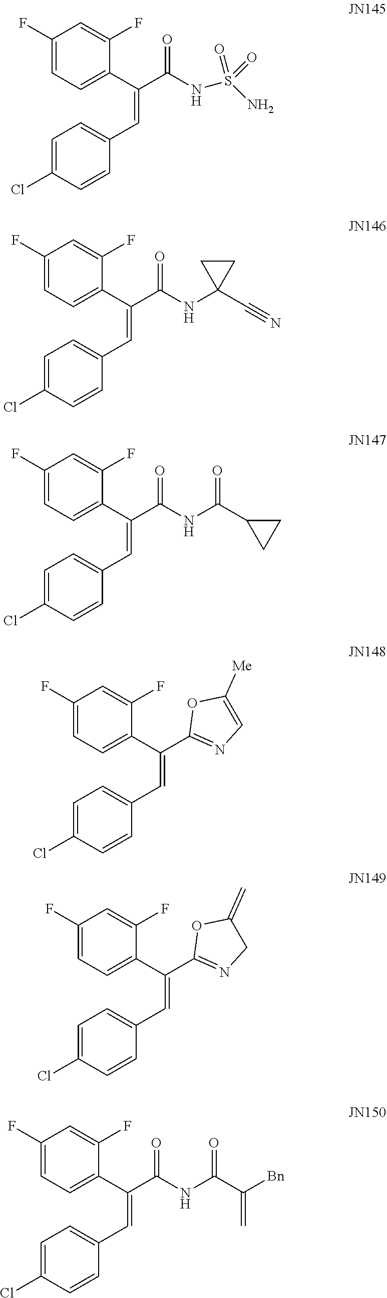 [SMILES: NS(=O)(=O)NC(=O)/C(=C/c1ccc(Cl)cc1)c1ccc(F)cc1F ; N#CC1(NC(=O)/C(=C/c2ccc(Cl)cc2)c2ccc(F)cc2F)CC1 ; O=C(NC(=O)C1CC1)/C(=C/c1ccc(Cl)cc1)c1ccc(F)cc1F ; Cc1cnc(/C(=C/c2ccc(Cl)cc2)c2ccc(F)cc2F)o1 ; C=C1CN=C(/C(=C/c2ccc(Cl)cc2)c2ccc(F)cc2F)O1 ; C=C(Cc1ccccc1)C(=O)NC(=O)/C(=C/c1ccc(Cl)cc1)c1ccc(F)cc1F]

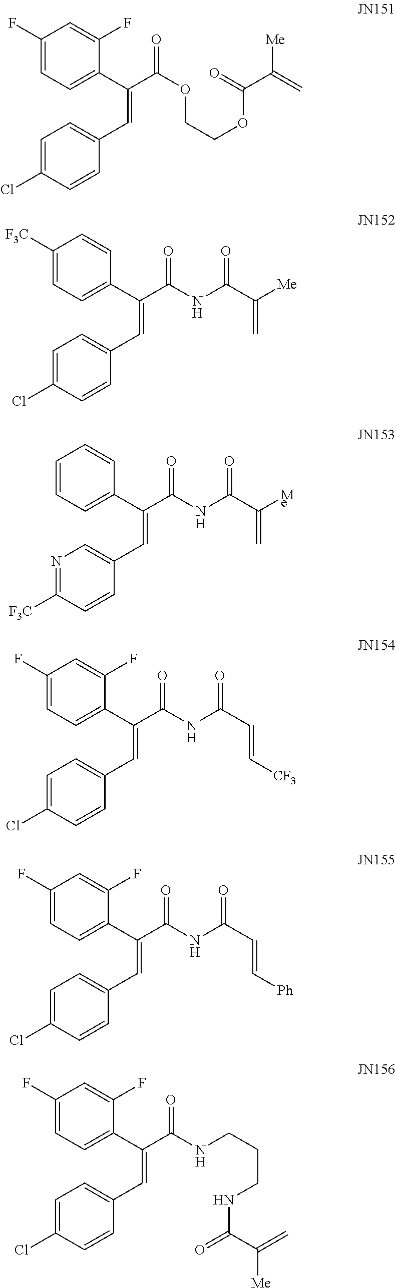

47. A solid form, which is Form I of compound JN032 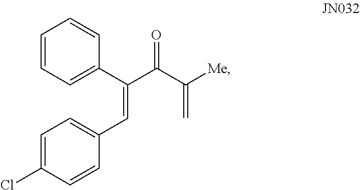 [SMILES: C=C(C)C(=O)/C(=C/c1ccc(Cl)cc1)c1ccccc1]
characterized by X-ray powder diffraction peaks at 2θ angles of about 21.5°, about 22.6°, and about 27.3°.

48. The solid form of claim 47, further characterized by X-ray powder diffraction peaks at 2θ angles of about 16.5°, about 20.5°, and about 28.2°.

49. The solid form of claim 47, characterized by an X-ray powder diffraction pattern substantially as shown in FIG. 4.

50. A solid form, which is Form I of compound JN110 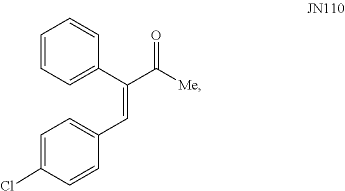 [SMILES: CC(=O)/C(=C/c1ccc(Cl)cc1)c1ccccc1]
characterized by X-ray powder diffraction peaks at 2θ angles of about 17.6°, about 22.2°, and about 28.8°.

51. The solid form of claim 50, further characterized by X-ray powder diffraction peaks at 2θ angles of about 10.2°, about 15.0°, and about 21.3°.

52. The solid form of claim 50, characterized by an X-ray powder diffraction pattern substantially as shown in FIG. 5.

53. A solid form, which is Form I of compound JN034 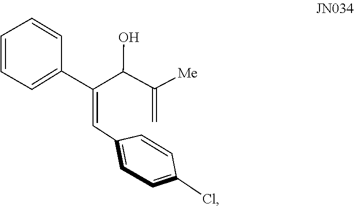
characterized by X-ray powder diffraction peaks at 2θ angles of about 8.3°, about 17.7°, and about 22.4°.

54. The solid form of claim 53, further characterized by X-ray powder diffraction peaks at 2θ angles of about 9.7°, about 14.4°, and about 25.0°.

55. The solid form of claim 53, characterized by an X-ray powder diffraction pattern substantially as shown in FIG. 6.

56. A solid form, which is Form I of compound JN097 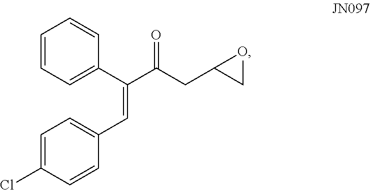 [SMILES: O=C(CC1CO1)/C(=C/c1ccc(Cl)cc1)c1ccccc1]
characterized by X-ray powder diffraction peaks at 2θ angles of about 20.5°, about 23.1°, and about 27.0°.

57. The solid form of claim 56, further characterized by X-ray powder diffraction peaks at 2θ angles of about 12.1°, about 18.7°, and about 22.1°.

58. The solid form of claim 56, characterized by an X-ray powder diffraction pattern substantially as shown in FIG. 7.

59. A solid form, which is Form I of compound JN117 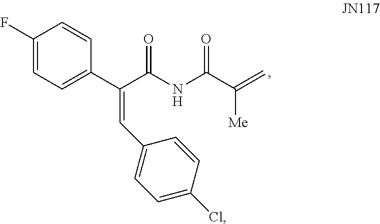 [SMILES: C=C(C)C(=O)NC(=O)/C(=C\c1ccc(Cl)cc1)c1ccc(F)cc1]
characterized by X-ray powder diffraction peaks at 2θ angles of about 7.8°, about 16.4°, and about 21.5°.

60. The solid form of claim 59, further characterized by X-ray powder diffraction peaks at 2θ angles of about 18.5°, about 19.1°, and about 20.1°.

61. The solid form of claim 59, characterized by an X-ray powder diffraction pattern substantially as shown in FIG. 8.

62. A solid form, which is Form I of compound JN103 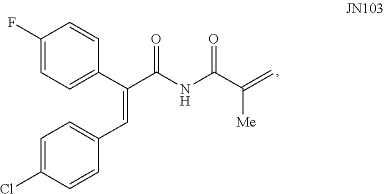 [SMILES: C=C(C)C(=O)NC(=O)/C(=C/c1ccc(Cl)cc1)c1ccc(F)cc1]
characterized by X-ray powder diffraction peaks at 2θ angles of about 6.6°, about 18.0°, and about 21.6°.

63. The solid form of claim 62, further characterized by X-ray powder diffraction peaks at 2θ angles of about 23.7°, about 25.1°, and about 28.1°.

64. The solid form of claim 62, characterized by an X-ray powder diffraction pattern substantially as shown in FIG. 9.

65. A pharmaceutical composition comprising the compound of any one of claims 1-64 and a pharmaceutically acceptable excipient.

66. Use of a compound or composition of any one of claims 1-65, for inhibiting an androgen receptor.

67. Use of a compound or composition of any one of claims 1-65, for inducing degradation of an androgen receptor in a cell expressing an androgen receptor.

68. Use of a compound or composition of any one of claims 1-65, for treating a mammal suffering from cancer.

69. The use of claim 68, wherein the cancer is prostate cancer.

70. The use claim 69, wherein the cancer is castration-resistant prostate cancer.

71. The use of any one of claims 68-70, wherein the cancer is metastatic.

72. The use of any one of claims 68-70, wherein the cancer is non-metastatic.

73. The use of any one of claims 68-72, wherein the cancer is resistant to antiandrogen therapy.

74. The use of claim 73, wherein the cancer is resistant to treatment with enzalutamide, bicalutamide, abiraterone, flutamide, nilutamide, darolutamide, or apalutamide.

75. The use of claim 73, wherein the cancer is resistant to treatment with enzalutamide, bicalutamide, abiraterone, flutamide, or nilutamide.

76. The use of claim 73, wherein the cancer is resistant to treatment with abiraterone acetate.

77. The use of claim 73, wherein the cancer is resistant to conjoint treatment with abiraterone acetate and prednisone.

78. The use of claim 73, wherein the cancer is resistant to conjoint treatment with abiraterone acetate and prednisolone.

79. A method of inhibiting an androgen receptor, comprising contacting the androgen receptor with a compound or composition of any one of claims 1-65.

80. A method of inducing degradation of an androgen receptor, comprising contacting the androgen receptor with a compound or composition of any one of claims 1-65.

81. A method of treating a mammal suffering from cancer, comprising administering a compound or composition of any one of claims 1-65.

82. The method of claim 81, wherein the cancer is prostate cancer.

83. The method of claim 82, wherein the cancer is castration-resistant prostate cancer.

84. The method of claim any one of claims 81-83, wherein the cancer is metastatic.

85. The method of any one of claims 81-82, wherein the cancer is non-metastatic.

86. The method of any one of claims 81-85, wherein the cancer is resistant to antiandrogen therapy.

87. The method of claim 86, wherein the cancer is resistant to treatment with enzalutamide, bicalutamide, abiraterone, flutamide, nilutamide, darolutamide, or apalutamide.

88. The method of claim 86, wherein the cancer is resistant to treatment with enzalutamide, bicalutamide, abiraterone, flutamide, or nilutamide.

89. The method of claim 86, wherein the cancer is resistant to treatment with abiraterone acetate.

90. The method of claim 86, wherein the cancer is resistant to conjoint treatment with abiraterone acetate and prednisone.

91. The method of claim 86, wherein the cancer is resistant to conjoint treatment with abiraterone acetate and prednisolone.
